SEQUENCE LISTING (75 sequences)

SEQ ID NO: 1 - protein, 191 aa, Arabidopsis thaliana
MATLDSPLEVLAFDYVNFVFNNLWTWIAVVTAAVSFWRIRATTTTTTSGGGRDNGLIDESFLEPPKPQATKAALLMETKPPRVKVTETEDWSLLLCKDGVTKGKLTVYYEEEIDGEREEDDGETTAVKYGGGESGEWWERWERVVKMRNGDEGWYRYVDLTVINGNVVRLWDANRVRNGGWVSVQRKECYG

SEQ ID NO: 2 - DNA, 857 bp, Arabidopsis thaliana
  source 1..857: mol_type = other DNA
gcatctatgaaagatccaccgttcaacttgtcaagatccaatggctcaccatctatctctcttcagatccatccaacggctaagattcaatctctccccatactaatcctctctttctttctccctttatataaccactgattcaatcttcttcgttaggatataataaaaacatttcagaaatggcgactttggattctccgttagaggttttggctttcgactacgttaacttcgtctttaacaatctttggacatggatcgccgtcgtgacggccgccgttagtttctggcggatccgagccactaccactaccaccaccagcggaggtggtagagacaatggcttaatagatgaatcttttcttgagccaccaaaaccacaagcgacaaaggctgctctccttatggagacgaaacctcctagagtcaaggtaacggagactgaggattggagtttgttgttgtgtaaggacggagtaacgaaggggaagctaaccgtgtactacgaagaagagattgacggagagagagaagaagatgacggagagacaacggccgttaagtatggaggaggtgagagtggagaatggtgggagagatgggagagagtggtgaagatgagaaatggagatgaaggttggtaccgttacgtggatttaacggtgattaacggaaatgttgtgaggttgtgggatgctaaccgtgtacgtaacggtggttgggttagtgtgcaacgtaaggagtgttatggttgagacatgttggggacaagtataattttctaatttaagcatatgattattcattgaacctttttggtttaaagactatttttgttgaattaaagttgtaat

SEQ ID NO: 3 - DNA, 744 bp, Arabidopsis thaliana
  source 1..744: mol_type = other DNA
attatacacacttcaataaagagatcgatgtcgactttggaatctccattagaggcgttggcgtttgaatacgctagcttaggtgttttcgccgtcgtcaacaacgtctggacatggatcgccgtcgtgactgccgctgtcagcttctggaggatcagagtcacaaccataggagtcggagacggccatgcatgtgtcttgatagaagaattaaccggttctaaatctgaaaacgaatccggtcgtctcgaaccgaaatcaataaccggtccggtcaaagaaacggttgcacgagtgaaggaaacggttacgaaaacggagccgttaatatgcgatgacggagtgacaaaggggaagctgacgatgtgctacgaggtagacgttgacgttgacggtgggaggtgtgttaacggagatttaacggcagttagctacggaggaggtttgggtaattgtggcggggattggtgggagaaatgggatggagtggtgaggatgagaaatggtgatgacagttggtaccgttacgtggatttaacggtgattaatggaaatgtggtaaggttatgggatgacaacaaaacactagtaacggcggcatgtgtctaaattagagaagtttcatatttcggaaagtttttaaatcttgagaagctttcttggtttgaagtgttttttttttgttggttgattaagttgtaatttgtaaataattttcacacaagagaccaagaaggaacgctt

SEQ ID NO: 4 - protein, 193 aa, Arabidopsis thaliana
MSTLESPLEALAFEYASLGVFAVVNNVWTWIAVVTAAVSFWRIRVTTIGVGDGHACVLIEELTGSKSENESGRLEPKSITGPVKETVARVKETVTKTEPLICDDGVTKGKLTMCYEVDVDVDGGRCVNGDLTAVSYGGGLGNCGGDWWEKWDGVVRMRNGDDSWYRYVDLTVINGNVVRLWDDNKTLVTAACV

SEQ ID NO: 5 - protein, 139 aa, Arabidopsis thaliana
MSTLESPLEALAFEYASLGVFAVVNNVWTWIAVVTAAVSFWRIRVTTIGVGDGHACVLIEELTGSKSENESGRLEPKSITGPVKETVARVKETVTKTEPLICDDGVTKGKLTMCYEVDVDVDGGRCVNGDLTACFFFVG

SEQ ID NO: 6 - DNA, 939 bp, Brachypodium distachyon
  source 1..939: mol_type = other DNA
cctcatcacaccaaacccgctttgaaattctaatcctatctcgaaccaccgcccccaatcaatcaccgccgccgcgaatggagctcctggacgtggtgccggcggaagccatcgctctccgcctctactccctcaccgcggcggccaacaccgtcgtctcgctctgcgcctggctcgtcgcggccctcgccgccgccgccgtcggcctctggcgcgtccgcgcggccggctcctcacacaaacccggcggcgccgtagtccgaagtaccctcgtggacaacaagaagatagcatcggaatcctttgacggaccacggcctgctcggtccgagccggcgtccccaattagcgagccgagctcgccgtccaaggtccggttcacggcctactacggcgggacaggatctgacggcggcgacgatggagtagtggaaggcgtcaagaaatgcgcggagagggacgaggatgatttcaacggcgagagcgagacggctgtgctgagacggacggcgtcgatgaggatgaggtcgacgattaaggcgcccttgatggcggcgccggactgggaggagaaggagatggccctcagaaagaggggcgatttgggttggtaccgtcacctcgacatggcggtgctcgatggcagcgtcgtgaggctctggaccggcgaggtcaccgcggcggtgcaggcctcgccgagggagcggcggagggcaggattggaactgcacttgtcagtatagagactggattataatgtgtatattgtggtgtaggattactggattgtgattagggcggtatgacaattttgtcctttgggcatcagggcatgtcatatctttgaactttgaagagggccaaggctgaaatgaacagccctcgccccaggacataaatgaaaactggcaatgccagtgtattctctcccagtttctt

SEQ ID NO: 7 - protein, 221 aa, Brachypodium distachyon
MELLDVVPAEAIALRLYSLTAAANTVVSLCAWLVAALAAAAVGLWRVRAAGSSHKPGGAVVRSTLVDNKKIASESFDGPRPARSEPASPISEPSSPSKVRFTAYYGGTGSDGGDDGVVEGVKKCAERDEDDFNGESETAVLRRTASMRMRSTIKAPLMAAPDWEEKEMALRKRGDLGWYRHLDMAVLDGSVVRLWTGEVTAAVQASPRERRRAGLELHLSV

SEQ ID NO: 8 - DNA, 1141 bp, Medicago truncatula
  source 1..1141: mol_type = other DNA
catcttattagagatcccacccaccctcatttctctcttcctcccactccttatatcttcagcctcctcaatccatttctcttattcaattcattcctctcaaacacacacacaccacaaaactaaaccaaaacaataaagataatgaacctcttgttagattccaacgttgaggctttagccttcaactacttaaactttggtttaatcacaattctcaacaacttatggacttggcttgctatcaccgccgctctcagcttctggaagattcgctcatccggttgtcccaaatcactagacccagtttcagtaaaaccggatcagagtgtctcagtttccaacgttacatcaccggtggaaataaaacccgacgagagtgtcttgatttccactgaaaatgtaacgtcaaaaactgagacaccaccaacattatcagatgtttctgatgatgttgatggtgtaaggaaggggaagtttaccttgtattacaaggaggacatgcaatgtggattcaacaagaatagtagtaattgttaccggcaattgccggttgttgaagggtgggaaccggaagttgaagttgagtggtggaagtgttgggagaaggtgttaaggttgagaaatggagagaatgagaatgggtggtacacgtgtcaagatttaacggagcttaatggaaacgtggttaaaatttgggacggtgggttaacatttgttggtagttgcatcacaaatgaatcatggtccagttccagatgtatgtcttttgaatgataataatgagaaaggaaaaaaaaaaaaagtagaaatagcttttttttttattaggtgtagttaggtttatgtcgtgtttgtttgtgttggattaataataatctctccactaaagatttgagagattatttagatttgagaatgtatatatggtaatttggtagtgcggtttggaaattttggatgatgaaattgattattctcaaatttgaaatgaaattggccaaaagcaacaagctttgcgtatgtctttgaatctttccttctatctcatgtcataattttcttttttaaggttttctaccttatgtcattattatctatttttttttattttttttatttgttagaagaaaaaagtttg

SEQ ID NO: 9 - protein, 210 aa, Medicago truncatula
MNLLLDSNVEALAFNYLNFGLITILNNLWTWLAITAALSFWKIRSSGCPKSLDPVSVKPDQSVSVSNVTSPVEIKPDESVLISTENVTSKTETPPTLSDVSDDVDGVRKGKFTLYYKEDMQCGFNKNSSNCYRQLPVVEGWEPEVEVEWWKCWEKVLRLRNGENENGWYTCQDLTELNGNVVKIWDGGLTFVGSCITNESWSSSRCMSFE

SEQ ID NO: 10 - DNA, 973 bp, Medicago truncatula
  source 1..973: mol_type = other DNA
cccatattcccattgacggtccccactcacctgtgttatagttcttctcatatcactctttcttctcttctcttctccataaataaattcttctttcaccactctcactcacaccccctccactcttaccatcaaaagtgaaaagaagagaagaacacgctcacagagatgaacatgttagaaaattctccacttgacactctagctttcaactacttgagcttcgatttcttcaaccatttatggacatggctcgccgtcatcttctggaggatcccaacccctaaacctgaattgttacctccttcccacgacacttcctccgataaacctcacctaggtctggaagtgttggaacctcgtcatgattatgcttgtccagcgcgtgttcctagtaacagcgttgttgaagacgatgacggagttacaaagggaaagatgaagtttaccttgtattattatgaccatgaagatgaagatgacattgacagagaatgcaaggagagtgttgagacgttaaaaaaaataacggaggaattatgggaagagaaagaaggaagattagggtggtgggagagttgggaaaacttgttgaggacaagaactggagagaacgaaggtgggtggtatacatgccaagatttaacggtgattaacggcaacgttgttaggttttgggatgaagagttcgagtttgcttcctttgcaaatggaggaatcaaataacagcttcagctgtatgttcttgtatggtagtaatcactaattagattagatagagaaacataagtatgttataataatattgttatcatcaatgtatatatggtggtgggttaattaatttggataatatatatcatgataagttgatatataatcatctcctcattcatgctctttggctctcaaatttgtaacgcaaagtgttgatatagagcaaccaattaattctccgtacattttttctgta

SEQ ID NO: 11 - protein, 185 aa, Medicago truncatula
MNMLENSPLDTLAFNYLSFDFFNHLWTWLAVIFWRIPTPKPELLPPSHDTSSDKPHLGLEVLEPRHDYACPARVPSNSVVEDDDGVTKGKMKFTLYYYDHEDEDDIDRECKESVETLKKITEELWEEKEGRLGWWESWENLLRTRTGENEGGWYTCQDLTVINGNVVRFWDEEFEFASFANGGIK

SEQ ID NO: 12 - DNA, 1091 bp, Glycine max
  source 1..1091: mol_type = other DNA
taggttccgattctggcatctttattagagatccctacctcacttgtcgcttctcccaccctttaaatcctttcctttcctctcacacaacacaaaaattgttaaattcccaatccaatagttttattagttcataaaaggaaaaagaaaatatgaatgtcttggattctaatgtggaagctctagccttcaactacttgagcttcggttttctcactgccctcaacaacctatggacctggctcgctctcttaaccgccgccctcagcttctggaagattcgctccgcgggctgccccaagcccaggcccaagaccgaggcccaagcccaaccttttttcaggcctgaggctgtcgcggatttgttaatagtggagaaaacggagccgacgaaacaacatgcagcgacggctccgttttctccaccgttattacgaggagaaaataacagtgaagaaaacggcgagaggaaggggaagtttaccgtttattacgaggaggaggacatgggcatgcaatgcacgtgcgatgagaatgaggggttgttaacggcgtggaaagagaaagaggggaccgaaacggagtggtggaataggtgggagaggttgttgcagttgagaaatggggagagcgagaacgggtggtacaggtggcaggacttgacggagcttaacggcagcgtcgttaggatatgggacggtgggttaagtggttcttccgttagggaatcgtggtacaacaactacaactctagctgtatgcatgtctggtaatgataataagaatagtgatgaaaaataatagtccttgtgttggattttaactattaagcttctctccttcaagactttggagaggttgttgagatttgagaatttgtatatatatggtattttggtagtggatatgcaattttttatgaagaaattatgaagtagagcaataaattaattaagcatcatcgttgttgtcgttcacgcatttgggttttaatatttgacacacaaatgctcgaaatcgagctttgtttatgttttttcttaggcttgtttatgttttttaatttgtatttatcggtcattattgtttct

SEQ ID NO: 13 - protein, 206 aa, Glycine max
MNVLDSNVEALAFNYLSFGFLTALNNLWTWLALLTAALSFWKIRSAGCPKPRPKTEAQAQPFFRPEAVADLLIVEKTEPTKQHAATAPFSPPLLRGENNSEENGERKGKFTVYYEEEDMGMQCTCDENEGLLTAWKEKEGTETEWWNRWERLLQLRNGESENGWYRWQDLTELNGSVVRIWDGGLSGSSVRESWYNNYNSSCMHVW

SEQ ID NO: 14 - DNA, 823 bp, Glycine max
  source 1..823: mol_type = other DNA
aattgaattgaggaggcatcttaggttgtcgattcggagcatcttatgtgagatcccatattctccaaccatataataaccttcaccacatttctctctcgcattcaattaattcaacatacacgtcatgaacggtgtttctcctctagaggcccaagccttcaactacctgagcttcggtttcctcaccctcctcaacaatttctggacatggctcgccttcactttctggaggacccgagcccccacctccgagttgctgccaccacccgatgacccggttcgcgacgagtcggatcctgttgccgtgaccgggccaagtccaactgtggttgacgttgacggtgcgaggaaggggaaatttaccttgtattacgaggatgagtctgagagggaatgcgagagccaccaggagacgacggcgctaacggagagagacggaagaggcctgaagtggtgggagagttgggaagagttgttgaaaataagacgaggagaagaccagaacggatggtacacgtgccaggatttaacggtgcttaacggcagcgttgttaggttatgggatggtggcttcggctttggtacctcaccacagaacccagggacactacaagatgtgttcttgtatggtaaattatataatagcatatctaccgatatttattagcttaagactgttagattatatattattcagaatatgtgtatatggtaatttgctagtagtacttgtttggatgatgataattaatgatcatcgtgtatgataatttgaaacgcaaatgatcaaagcaatcttaatttgcgtactttttttttt

SEQ ID NO: 15 - protein, 180 aa, Glycine max
MNGVSPLEAQAFNYLSFGFLTLLNNFWTWLAFTFWRTRAPTSELLPPPDDPVRDESDPVAVTGPSPTVVDVDGARKGKFTLYYEDESERECESHQETTALTERDGRGLKWWESWEELLKIRRGEDQNGWYTCQDLTVLNGSVVRLWDGGFGFGTSPQNPGTLQDVFLYGKLYNSISTDIY

SEQ ID NO: 16 - DNA, 771 bp, Glycine max
  source 1..771: mol_type = other DNA
gtggaatgaaatgggatgtagttgaattgaagaggcatcttaggtgggcgattcggagcatcttatgtgagatcccatattcttcaaccttactataataataaccttcaccacatttctctcgcgtattcaacatcatgaacgctgtttctcctctagaggcccaagccttcaactacttgagcttcggcttcctcagcaatttctggacctggctcgccctcactttctggaggacccgaactcccacctccgagttgctcccaccacccgacgacccgattcacgacgagtcggagtcggaccctgttgccgtaccggcgcgtgttccaattccatttgccgttgacgttgacggagccaggaaggggaagtttaccttgtgttacgaggatgagtttgagagggaatgcgagacgacggtaacagaggagagagacggaagaggccgggagtggtgggagagttgggaaaccttgttgaaactgagacgaggagagaaccagaatggctggtacacttgccaggatttaacggcgcttaacggcagcgttgttaggttatgggatggtaccttcaccacacaacccagtgacaacacaagctgtgttcttgtatggtaaaatttggtaatactatagctagcagaaataatattagcttagattgtttccactgttggattactattgagagaatgtatatatggtagtaatttgctactagtcgttgtttggataatgatcatcttgtatggtactaatttcaatc

SEQ ID NO: 17 - protein, 161 aa, Glycine max
MNAVSPLEAQAFNYLSFGFLSNFWTWLALTFWRTRTPTSELLPPPDDPIHDESESDPVAVPARVPIPFAVDVDGARKGKFTLCYEDEFERECETTVTEERDGRGREWWESWETLLKLRRGENQNGWYTCQDLTALNGSVVRLWDGTFTTQPSDNTSCVLVW

SEQ ID NO: 18 - DNA, 669 bp, Zea mays
  source 1..669: mol_type = other DNA
atggagctcctcggcctggtgcccgcggaggccatagcgctccgcctctactccctccccgcagcagccgcggccgccggctcgctctacgcctggctcgtcgccgcactcgccgccgccatcggcctctggcgcatccgcgccgtcagcgtttccaacatcaacaagcgtgacgcaggcgtcagcgccctcgtcgacgataaccaggagccactggtgcctcttctgccgtctcggggggctgccattactgacgagccgcccgccgccgagccgtcttccgtgagcgagccgagcacgccgtccaaggttcggttcactgcgtactacggtggctcgggagacgaagacggagtggtcgacggcgtcaggaaatgcgcggatgacgacgacgtcgacgacggtgcgcgcggccgagactgcgcggtggaggtggttctcagaaggaccgtgtcagagccggggacaagaagagcggcggccctggccacggggccgtgggaagggagggagatggcggtgaggcggaggagtgacctgggttggtaccgccacatcgacatggccgcgctcgacggcagcgtcgtgaggctgtgggacggcgacctgacggcgtcgccgagggggcggatgaggaggtcaggattggaattgcaattgccattatag

SEQ ID NO: 19 - protein, 222 aa, unidentified
MELLGLVPAEAIALRLYSLPAAAAAAGSLYAWLVAALAAAIGLWRIRAVSVSNINKRDAGVSALVDDNQEPLVPLLPSRGAAITDEPPAAEPSSVSEPSTPSKVRFTAYYGGSGDEDGVVDGVRKCADDDDVDDGARGRDCAVEVVLRRTVSEPGTRRAAALATGPWEGREMAVRRRSDLGWYRHIDMAALDGSVVRLWDGDLTASPRGRMRRSGLELQLPL

SEQ ID NO: 20 - DNA, 1125 bp, Hordeum vulgare
  source 1..1125: mol_type = other DNA
gtgtatcagtgcacttgtttacctaatccaacattcactaaatatatagaaattgcaaaagctcgcagtactaaactgcaaattccatcatttccattccgccacttatatgtacacttcctacccaagcccaacctcctcactatcctcatcgcaacaaccaaacttgctcctttctatctcgaacaaccgccgagagacctccccgtcgccgccgccgccgcaaatggagctgctcaacgtggtgccggcgggcgccatggacttccccctctactccctccccgcggcggccaacaccgtcgcctcgctcttcgcctggctcgtcgcggcactcgccgccgccgtcggcctctggcgcatgcgcgcggtcggctcctccagcaaagcacccggctctggcgaccgcagcagcaccctcgtggaaaacaagcagccgctgcaggctgtttcctcgcctgccgctgacgagtcacgacccagactcgccgagccggtggagccggcttccccgatgagcgagccgagctcgccgtccaaggtccggttcacggcgtactacggcggggccggagctgacgccggcgatgacggagtagtggatggcgtcaggaaatgcgcggacagggacgatgacgacgacagcggtgtgcatgccgtcgatgacctgagcgagacgctgtcgagacggacggcgtcgatgaggatcaggccggcggtgccctgctgggaggagagggagatggccgtgaggaggcgggcagatctgggctggtaccgccaccttgacatgacggtggtcgacggcaccgttgtaagactgtgggacggcgaggtcaccgcgtcggtggcgtcgccgagggcgcggcgggggagggcaggattggaactgcgcctgttactatagagaaggagatcctaatgtgtgtataatgtggttaggattattggaatgttattatggtgggtatgacaattttgtccttcaatttgggcatgtcatgtgtatgtgtatcattgaaagagggccgaggctgaataaatgaacagccttcgtctcagctctcaggagaaatgaaaatttggcaaatggcagagtattctttgctcagttctctgcttcca

SEQ ID NO: 21 - protein, 265 aa, Hordeum vulgare
MYTSYPSPTSSLSSSQQPNLLLSISNNRRETSPSPPPPQMELLNVVPAGAMDFPLYSLPAAANTVASLFAWLVAALAAAVGLWRMRAVGSSSKAPGSGDRSSTLVENKQPLQAVSSPAADESRPRLAEPVEPASPMSEPSSPSKVRFTAYYGGAGADAGDDGVVDGVRKCADRDDDDDSGVHAVDDLSETLSRRTASMRIRPAVPCWEEREMAVRRRADLGWYRHLDMTVVDGTVVRLWDGEVTASVASPRARRGRAGLELRLLL

SEQ ID NO: 22 - DNA, 696 bp, Oryza sativa
  source 1..696: mol_type = other DNA
atggagctcctcgacatggtgcccgcggacgccatcgcgctccgcctctactccctcccggcggcggcggcggccgtcggctcgctctgggcgtggctcgtcgccgccctcgccgccgccgtcggcctgtggcgcatccgcgccgccgccggtgtccgtagcgccctcgtggacgacgacgactacaagcagcgcaaggcgaagcagccgcgcggtgctctgcggcctgccggcgtcggcgaggcacggccggctcgcgccgaggcggcggagtcggaggcgacgacgccgacctccccgagcgagccgagcacgccttccaaggtccggttcacggcgtactacggcggcgagggggacggcgccgacgagggagtcgtggacagcgtcaggagatgcgtggacaacgacggcgacggcgagggggagacgccgacggcgccggtgaggcggacggcgtcggggaggaggaggtggtcgacgacgacgacgacgacgacggcgccattcatggcgacgccgtgggaggagagggagatggccgtgaggaggaggggagacctggggtggtaccgccacctcgacatggcggcgctcgacggaagcgtcgtccggctgtgggacggcgaggtcacggcggcgtcgccggggcggcgcggccggagggcattatcggaattgcacctctcattgtag

SEQ ID NO: 23 - protein, 231 aa, Oryza sativa
MELLDMVPADAIALRLYSLPAAAAAVGSLWAWLVAALAAAVGLWRIRAAAGVRSALVDDDDYKQRKAKQPRGALRPAGVGEARPARAEAAESEATTPTSPSEPSTPSKVRFTAYYGGEGDGADEGVVDSVRRCVDNDGDGEGETPTAPVRRTASGRRRWSTTTTTTTAPFMATPWEEREMAVRRRGDLGWYRHLDMAALDGSVVRLWDGEVTAASPGRRGRRALSELHLSL

SEQ ID NO: 24 - DNA, 1039 bp, Panicum virgatum
  source 1..1039: mol_type = other DNA
cctcctcctctctctctctctctctctctctctctctctctctctctctctctctctctctctccccagtctccaacaaccgccccgccgcgaatggagctcctcgacatggtgcccgcggacgccatcgcgctccgcctctactccctcccggcagcggcggcggcctcgctctactacgcctggctcgtcgccgcgctcgccgccgccgtcggcctgtggcgcatccgcgcggtcggcgccggcgtcagcagagccagcgccgtcgtcgtcgtcggagaaaagcagcaggcggcccagccgtcgcccgccgttgaggagccgcggccggcggcgcgggcgcccgagccggctgatgaggcggcgtcccggagcgagccagagtcgagcacgccgtccaaggtccggttcacggcgtactacggtgtttccggaggcgacgacggcggagtggtggacagtgtcaggagatgcgcggacgaggacgaggacgaggacgacgaggggatcgatggcgagatggaggcggtcctgaggaggacggcgtcggcgccggagagaagaagggcggcgacgaccttcgcggtggcactgtgggaggagagggagatggccgtgaggcggaggggcgacctggggtggtaccggcacctcgacatggcggcgctcgacggcagcgtcgtgaggctgtgggacggcgagctgacggcgtcgccgagagcgcggtggaggagggcaggattggaattacagttgtcattttagagatggtgtatgcttaatctgtatattgtggttgtattgtaggagtaatgggatgatgttattaggaggatatggcaattttgccctttgagattgtcatcaagttatggccaaggccaaagggcaaagaaggtccctttgtcactagtcctgtacccgagccagggcacaaatggaaactgataaatgtcagtggtatctatcattctacctccgacctccctcctacctcaggccctttgcatccattgtgtcttctaccattctgcctttt

SEQ ID NO: 25 - protein, 223 aa, Panicum virgatum
MELLDMVPADAIALRLYSLPAAAAASLYYAWLVAALAAAVGLWRIRAVGAGVSRASAVVVVGEKQQAAQPSPAVEEPRPAARAPEPADEAASRSEPESSTPSKVRFTAYYGVSGGDDGGVVDSVRRCADEDEDEDDEGIDGEMEAVLRRTASAPERRRAATTFAVALWEEREMAVRRRGDLGWYRHLDMAALDGSVVRLWDGELTASPRARWRRAGLELQLSF

SEQ ID NO: 26 - DNA, 1102 bp, Setaria viridis
  source 1..1102: mol_type = other DNA
tccattcctccacaccgcaccagggtcatcccccacctcctctctcgctctccaaccaccgccgagacctccccaccaccacttaccatcgaaacgccgcgaatggagctcctcgacatggtgcccgcggacgccatcgcgctgcgcctctactccctccccgcagcggtagccgccgccgcctcgctgtactacgcctggctcgtcgccgcgctcgctgccgccgtaggtctctggcgcatccgcgcggtcagcgccggcgttcgcagaggcagcgccatcgtcgatgacaagtcgaaggcccagtctccttcgccgtctcctgccatcgaagagccgcggctggcggctccggccgagccggcgtccccgagcggcgagccgagcacgccgtccaaggtccggttcacggtgtactacggtgtttcgggagacgccaacgacggagttgtggacggcgttcggagatgcgcggacgacgacgacagggtcgacggcgaggtagacgcggtcctgcgaaggacggcgtcggcgccggagagaagaagggcgaaggccctggcggcggcggcgccgtgggaggagagggagatggccgtgaggcggaggggcgacctggggtggtaccgccacctcgacatggcggcgctcgacggcagcgtcgtgaagctgtgggacggcgagctgacggcctcgccgagagcgcggaggaggagggcaggattggaattacagttgtcattttagagatggcatatggttaatctgtatattgttgttgtaggagtaatggaatgatgttattaggtggatatggcaattttgccctttgatattgtcgtaagttatggccaaggccaaagggcgaagatgggctctttgtgtgcgatggcacaattggaaactgataaatgtcagtggtatcctagctcagatccttgcatccatggtgtctagctgctgctgctgctgcttcttcttcttttattttgattttcttgcatgtttacattagaagtggtgcttcagaacgcatttagttttatcggttgtactattttttttactgacctatcaattccagttccagtatgaagggaaa

SEQ ID NO: 27 - protein, 214 aa, Setaria viridis
MELLDMVPADAIALRLYSLPAAVAAAASLYYAWLVAALAAAVGLWRIRAVSAGVRRGSAIVDDKSKAQSPSPSPAIEEPRLAAPAEPASPSGEPSTPSKVRFTVYYGVSGDANDGVVDGVRRCADDDDRVDGEVDAVLRRTASAPERRRAKALAAAAPWEEREMAVRRRGDLGWYRHLDMAALDGSVVKLWDGELTASPRARRRRAGLELQLSF

SEQ ID NO: 28 - DNA, 1430 bp, Sorghum bicolor
  source 1..1430: mol_type = other DNA
cgcctgttcaatttcagttcggtaaattggtattacacagtctgagcgatactctgcctctgttccaatataccgtacaaattaaagatggatggaataggtgcactagctcaactccacaaaagactcaaatcatatacgaaattgttcatctaaaaattggaggctgaatgattgcaaaatgcagcttatactgcacccaagaaagaaaattggaaaatgctcagttgtgcaggtgaaagcctgcaataattccaggcagttcaccagtgcacattcgttcccactcccgccacctatatatacgcttcccacccaaacccatcgtcttccattcctcaccgcacaccaacaccatccccctcttcaacaaccgccgagacctcccccaccaacaccaccacaaccacaaccacaccgccgcgaatggagctcctcggcatggtgcccgcggaggccattgcgctgcgcctctactccctccccgcggcggccgtggccgcgggctcgctctgcgcctggctcgtcgccgcgctcgccgccgccgcggtcggcctctggcgcatccgcgccgtcagcggcgcttccaactccaaggccgccatcgccgccggtgttcgcagcgccagcgccctcgtcgacgacgataatcaggagccacaggcggcgcctctgcctccgtctcgggctgccattactgacgagccgcggcggccggtcgctctcgctacggcgcccgagccgtcgtcgtccgtgagcgagccgagcaccccatcgaaggttcggtacactgcgtactacggcggctcaggagacgacgacgacgaaggattggtggacggcgtcaggaaatgcgcggacgcggacgacgacggcgaggcggaggtggttctcagaagaacggtgtcagagccggcggggagaagaagagcgacgaccttggcggccacggcgccgtggaatgaagagagggagatggcggtgaggcggaggagcgacctgggttggtactcccacatcgacatggcagcgctggacggcagcgtcgtcaggctgtgggacggcgacctgacggcgtcgccgagagggccgcggaggagggcaggattggaattgcaactctcactgtaagagaggtagtgtggttaatgcgggcatgtaatgtatatgtggtagggacaatgggttgttagtaggaggatatgacgattttgggcataagattgtcataagttatagccagggcaaagagctcttcttcaatcgtgtgcttgaatatggtatggttcctcgctgctggtctggaaaagattagacttacgagaacacagtttaaagtttagcctctttggagcatgttcatttgtccgaaatgacatgactaaaagtattgttcgctgatttattatataagagaaaaacactcttgg

SEQ ID NO: 29 - protein, 234 aa, Sorghum bicolor
MELLGMVPAEAIALRLYSLPAAAVAAGSLCAWLVAALAAAAVGLWRIRAVSGASNSKAAIAAGVRSASALVDDDNQEPQAAPLPPSRAAITDEPRRPVALATAPEPSSSVSEPSTPSKVRYTAYYGGSGDDDDEGLVDGVRKCADADDDGEAEVVLRRTVSEPAGRRRATTLAATAPWNEEREMAVRRRSDLGWYSHIDMAALDGSVVRLWDGDLTASPRGPRRRAGLELQLSL

SEQ ID NO: 30 - DNA, 1080 bp, Triticum aestivum
  source 1..1080: mol_type = other DNA
ccctcctcactatcctcatcattcacaacaaactcacagaccaagcctcctccttcctatcttgaacaaccgccgggaggcctccccgtcgccgtcgccgcaaatggagctgctcaacgtggtgccggcggactccatggccttccccctctactccctccccgcggcggccaacaccgtcgcctcgctcttcgcctggctcgtcgccgccctcgccgccgccgttggcctctggcgcatccgcgcggtcggctcctccaacaaactacccgtcgccggcgcccgcgcccacggcagcaccctcgtggacgacaagcagcagacgcaggccgtttcgtcgcctgccgctgacgagccacggcccagactcaccgagccggtggagccggcttccccgattagcgagccgagctcgccgtccaaggtccggttcacggcgtactacggcggggccggagctgacgccggcgacgacggagtagttgacggcgtcaggaaatgcgccgacagggacgaggacgacaacggcgtgcccttcgtcgacgaccagagcgagacgctgccgagacggacggcgtcgatgaggatcaggtcggcggcctcgacggcggtgccctgctgggaggagagggagatggccgtgaggaggcggggggatctgggctggtaccgccaccttgacatggcggtgctcgacggcaccgtcgtaaggctgtgggacggcgaggtcaccgcggcggtggcgtcgccgagggcgcggcgggggagggcaggattggaactgcacctgtcactatagagatggagatcctgatgtgtataatgtggttaggattgttggactgtgattaggtgggtatgacagttttgtccttcaatttgggcatgtcatatgtatgtgatgtgtatcatggaagagggccgaggctgaaaaatgaacagccttcgtctcagctctcaggagaaacgaaaatttggcaaataatggcagagtattcttgctcagttcccttccctgcttccatttccatggcttgcactgctctccattacatccttctgacccttttgttctctcgg

SEQ ID NO: 31 - protein, 231 aa, Triticum aestivum
MELLNVVPADSMAFPLYSLPAAANTVASLFAWLVAALAAAVGLWRIRAVGSSNKLPVAGARAHGSTLVDDKQQTQAVSSPAADEPRPRLTEPVEPASPISEPSSPSKVRFTAYYGGAGADAGDDGVVDGVRKCADRDEDDNGVPFVDDQSETLPRRTASMRIRSAASTAVPCWEEREMAVRRRGDLGWYRHLDMAVLDGTVVRLWDGEVTAAVASPRARRGRAGLELHLSL

SEQ ID NO: 32 - DNA, 995 bp, Triticum aestivum
  source 1..995: mol_type = other DNA
ctcctcactatcctcatcattcacaacaaacccaaaaaccaaacctgctccttccctatcttgaacaaccgcccggagacctccccgtcgccgtcgccgtcaccgcaaatggatctgctcaatgtggtgccggcggacgccatggccttccccctctactccctccccgcggcggccaacaccgtcgcctcgctcttcgcctggctcgtcgcggccctcgccgccgccgtcggcctctggcgcatccgcgcggtcggctcctccaacaaactacccgacgccggcgcccgcagcagcaccctcgtggacgacaagcagcagatgcaggctctttcgtcgcctgccgctgacgagccacgacccgcacgcaccgtgccggtggagccggcttcccctattagcgagccgagctcgccgtccaaggtccggttcacggcgtactacggcggggccggagctgacgccggcaacgatggagtagtggatggcgtcaggaaatgcgcggacagggacgaggacgaggacggcgtgcccgtcgtcgacgaccagagcgagacgctgccgagacggacggcgtcgatgaggatcaggtcggtggcatcgacggcggtgccctgctgggaggagagggagatggcggtgaggaggcggggagatttgggctggtaccgccaccttgacatggcagtgctcgacggcaccgtcgtaaggctgtgggacagcgaggtcaccgcggcggtggcgtcgccgagggcgcggcgggggagggcaggattggaactgcacctgtcactatagagatggagatcctgatgtgtataatgtggttaggattattggactgtgattaggtgggtatgacagttttgtccttcaatttgggcatgtaatatgtatgtgtatcatcgaagagggccgaggctgaaaaaatgaacagccttcgtctcagctctcaggagaaataaaaatttggcaaatggcagagtattcttact

SEQ ID NO: 33 - protein, 229 aa, Triticum aestivum
MDLLNVVPADAMAFPLYSLPAAANTVASLFAWLVAALAAAVGLWRIRAVGSSNKLPDAGARSSTLVDDKQQMQALSSPAADEPRPARTVPVEPASPISEPSSPSKVRFTAYYGGAGADAGNDGVVDGVRKCADRDEDEDGVPVVDDQSETLPRRTASMRIRSVASTAVPCWEEREMAVRRRGDLGWYRHLDMAVLDGTVVRLWDSEVTAAVASPRARRGRAGLELHLSL

SEQ ID NO: 34 - DNA, 975 bp, Triticum aestivum
  source 1..975: mol_type = other DNA
accctcctcactatcctcatcattcacaacaaacccaaaaaccaaacctcctccttcctatcttgaacaaccgccgagagacctccccgtcgccgtcgccgcaaatggagctgctcaacgtggttccggcggacgccatggccttccccctctactccctccccgcggcggccaacaccgtcgcctcgctcttcgcctggctcgtcgcggccctcgccgccgccgtcggcctctggcgcatccgcgcggccggctcctccaacaaactacccggcgccggcgcccgcagcagcaccctcttggacgacaatcagcagccgcaggccgtttcgtcgtctgccgccgacgagccacggcccgcactcgccgtgccggtggagccgtcctccccgattagcgagccgagctcgccgtccaaggtccggttcacggcatactacggcggggccggagctgacgatggggtagtggacggcgtcaggaaatgcgcggacagggacgaggacgaggacggcgtgcccgtcgtcgacgaccagagcgagacacagccgagacggacggcgtcgatgaggatcaggtcggcagcctcgacggcggtgccctgctgggagaagagggagatggccgtgaggaggcggggagatttgggctggtaccgccaccttgacacggcggtgcttgacggcaccgttgtaaggctgtgggacggcgaggtcaccgcggcggtggcgtcgccgagggcgcggcggcggagggcaggattgaaaccgcgcctgtcactatagagatgtgtgtataatgtggttaggattattggattgttattaggtgggtatgacgattttgtccttcaatttgggcatgtcatatgtatgtgtatcgtctaagagggccgaggctgaaaaaatgaacagccttcgtctcagctctcaggagaaacgaaaatttggcaaataatggcagagtattcttgct

SEQ ID NO: 35 - protein, 226 aa, Triticum aestivum
MELLNVVPADAMAFPLYSLPAAANTVASLFAWLVAALAAAVGLWRIRAAGSSNKLPGAGARSSTLLDDNQQPQAVSSSAADEPRPALAVPVEPSSPISEPSSPSKVRFTAYYGGAGADDGVVDGVRKCADRDEDEDGVPVVDDQSETQPRRTASMRIRSAASTAVPCWEKREMAVRRRGDLGWYRHLDTAVLDGTVVRLWDGEVTAAVASPRARRRRAGLKPRLSL

SEQ ID NO: 36 - DNA, 2860 bp, Brachypodium distachyon
  source 1..2860: mol_type = other DNA
gatttgagcatgttcttgatgaggtccttggcgctgggggagatgttgggccacgggtcggagtcgaagtctatggcgccttttaggaccgcgtcgaagatcccctgctgcgtctcggcccagaagggcgggacgccggagagcaggatgtagacgatgacccccgccgtccagacgtcggcttcgggcccgtagtgcttgcagaggacctcgggggccacgtagtacgggcttccgacgacgtcggtgaagatctggcgggcttgaagaagacggagagtccgaaatcgatggccttgagatcggcgaccgagtcgtcttcgtcttctccgttgccggcgccggcgccgccgagcaagaggaagttctcgggcttgaggtcgcggtgcatgacccccagagaatggcacgcctcgacgacgccgacgacgacgcgtgcgatctcggcggctttccgctcggagaagtatccgcgggcgacgatgcggtcgaagagctcgccgccctcgcagaggtccatgacgatgtggacgtagagcgggtcctcgtaggcgccgcggatggtgacgacgctggcgtggcccgccaggtggtgcatgatctggatctcgcggcggacgtcgtccacgtcctcgggggtgaggagcttgcgcttggcgatggacttgcaggcgaggggtgtccccgtggcgatgtcggtgcagaggtaggtggtgccgaactggccctggccgagcttgcggccgagcgtgtagagggaggtgagcggcggggtgtcgtggccgaggacggcggtcggggaggagaggtggtgctggtggccgcgcatggtgttggtggtgcagggggcttggaggtggagatggaaggggtccgagtcggcggtgctgctgttggaatcgcggcacgagtagttgcccatgcgcaccgcgtcaattgtcgccggcggccatggcgaccaccgtggatggatgattggaccacagagaaattagggggtggagaggaagaggagagctgtgctccattagtttgggaggaagaggagaccaaattggcaatggcctgcatgtcgtgcgctgcacctacctaagctagcgtgcatgtcgatttgctcctgcgacaccacgattcggccctttttcggcctaaatgaaacatcgtccatctcgaatcaacctagccacatcattctttttctttttgcaagatcgatccctgtgcagtagacatgcatgctggagtagcagtaggaatcagggactggccagcctggccttgctagtgagcgagtgtacgtgcaatgccaattaaccgtttgcttattttactagtaccatcatatcgatcgatctcaatcaagctgctgacgtagggcaacatatataagatcgttttcagctcgtggtgcacgatgcgcaataataccgatcctgttagttgagttcaatcaattaagagctctgtttcctcatctctcacctacgagaagcggcgcatacagaaatagaagatgttgaggtagatcaagttcatattgatgttaacttgaatacttattgaagatttcaattcaaaggacactagaagaatgatgctgttcaaataaagatgttgaggtagaggaagttcattattctagtacttttctagtgagggagattttcgcacctgcatgtatttattgctgtcaaatatatgacgccaatgaaatagaaaaatactcttaattaataatatgcgataataaattattttaccccggccggtggtttatttttcttgcttcgcgcccctgcctagcgaggagaggtgcatgcgatccaccggcccatggatcgtcgcttaattagtaccggtaatttccttattaaaccaggaatgcaaataattcatgtcctggacagtgagatgatgagcaggtcggcgggtatgcgcgcgaacgtacggtctctgtcgatcgtgtgccacgtgcattagcggagccgacggcctgctcgcagagcccggacaaattccctaaaaattaattatacaagaaaaacactactctggtggctaattaacacgctggctagcggcatcatggcttccccagtgatcgatagcactggggaagcatgcatagctcgatggaatcactccatgcgagtgcatatgtcgcaccaaccaaatttctttcgtcacttagtatgaaacggagagaatgtatgatcgaccgattctgatcccgcatgataatagtgagatcgattctggtcccgcatgataataatgagatctcaacaaattaaccaacaaacatacaattgcacatgcctgcctatactacttatcaccgtccaaattaaagcattcatgccaccctagctaaaaatagatacatccatatttaaacaaatttgaattaagaatttagaaacgggagcaggcaggaacaatccagcggcttcttattgactctgtcaacacaacactagctagctgggttttcagacttcattaacagcgcacgctagcggcatcatggcttcccaagtgagcggtcgagcgccgacaaaaacgggaccccggccctctgtgtgatttgatgcgagttgctagcagtgtgtctgacactgtgatgtttggtccaggtatgaaccaaccaagatcacaggaaaaaaaacaatcgcacatgcatgtatgaatctcctccggcctatatatactcgccaccatctcggaattaaagcatgcatgccacttacagcaggcttgcatcaccagctgccactcagctgggttttcatcagtcttaaactgagctgtgttaattacctgagcacacacacagctcaagtctgaacaagctagtaag

SEQ ID NO: 37 - DNA, 22 bp, synthetic construct
  source 1..22: mol_type = other DNA
ggagactgaggattggagtttg

SEQ ID NO: 38 - DNA, 22 bp, synthetic construct
  source 1..22: mol_type = other DNA
tctctccgtcatcttcttctct

SEQ ID NO: 39 - DNA, 24 bp, synthetic construct
  source 1..24: mol_type = other DNA
atcttgagaagctttcttggtttg

SEQ ID NO: 40 - DNA, 20 bp, synthetic construct
  source 1..20: mol_type = other DNA
gcgttccttcttggtctctt

SEQ ID NO: 41 - DNA, 22 bp, synthetic construct
  source 1..22: mol_type = other DNA
cctcgtggacaacaagaagata

SEQ ID NO: 42 - DNA, 18 bp, synthetic construct
  source 1..18: mol_type = other DNA
agctcggctcgctaattg

SEQ ID NO: 43 - DNA, 21 bp, synthetic construct
  source 1..21: mol_type = other DNA
ttggtgacaacaggtcaagca

SEQ ID NO: 44 - DNA, 22 bp, synthetic construct
  source 1..22: mol_type = other DNA
aaacttgtcgctcaatgcaatc

SEQ ID NO: 45 - DNA, 20 bp, synthetic construct
  source 1..20: mol_type = other DNA
ggaggcacctcaggtcattt

SEQ ID NO: 46 - DNA, 20 bp, synthetic construct
  source 1..20: mol_type = other DNA
atagcggtcattgtcttgcg

SEQ ID NO: 47 - DNA, 20 bp, synthetic construct
  source 1..20: mol_type = other DNA
agaacccggaagcggataag

SEQ ID NO: 48 - DNA, 20 bp, synthetic construct
  source 1..20: mol_type = other DNA
gaagacagggttgcagaggt

SEQ ID NO: 49 - DNA, 20 bp, synthetic construct
  source 1..20: mol_type = other DNA
tactctcaggaccagctcaa

SEQ ID NO: 50 - DNA, 24 bp, synthetic construct
  source 1..24: mol_type = other DNA
caagatcacagttccctcactatc

SEQ ID NO: 51 - DNA, 22 bp, synthetic construct
  source 1..22: mol_type = other DNA
cgtccattgtactcctgcttac

SEQ ID NO: 52 - DNA, 20 bp, synthetic construct
  source 1..20: mol_type = other DNA
gcgtacaagctgatctcaca

SEQ ID NO: 53 - DNA, 20 bp, synthetic construct
  source 1..20: mol_type = other DNA
acctccacctcacttcttct

SEQ ID NO: 54 - DNA, 20 bp, synthetic construct
  source 1..20: mol_type = other DNA
ctgcggccattacttcttct

SEQ ID NO: 55 - DNA, 23 bp, synthetic construct
  source 1..23: mol_type = other DNA
aaggtgtagtttcagccatagga

SEQ ID NO: 56 - DNA, 23 bp, synthetic construct
  source 1..23: mol_type = other DNA
gatagtgggtcagaatcagttgc

SEQ ID NO: 57 - DNA, 25 bp, synthetic construct
  source 1..25: mol_type = other DNA
cacactacacacacgcaagtactgt

SEQ ID NO: 58 - DNA, 17 bp, synthetic construct
  source 1..17: mol_type = other DNA
cagcacgtcccccacaa

SEQ ID NO: 59 - DNA, 20 bp, synthetic construct
  source 1..20: mol_type = other DNA
tggttgtgatggtccgtttg

SEQ ID NO: 60 - DNA, 27 bp, synthetic construct
  source 1..27: mol_type = other DNA
agacagaaccgacttgctagaaattac

SEQ ID NO: 61 - DNA, 27 bp, synthetic construct
  source 1..27: mol_type = other DNA
agacagaaccgacttgctagaaattac

SEQ ID NO: 62 - DNA, 25 bp, synthetic construct
  source 1..25: mol_type = other DNA
cgaagaagattgaatcagtggttat

SEQ ID NO: 63 - DNA, 23 bp, synthetic construct
  source 1..23: mol_type = other DNA
caccttgctggtctcgggaattg

SEQ ID NO: 64 - DNA, 22 bp, synthetic construct
  source 1..22: mol_type = other DNA
ggtgaacgattcagtcggttat

SEQ ID NO: 65 - DNA, 23 bp, synthetic construct
  source 1..23: mol_type = other DNA
caccatggcgactttggattctc

SEQ ID NO: 66 - DNA, 19 bp, synthetic construct
  source 1..19: mol_type = other DNA
tcaaccataacactcctta

SEQ ID NO: 67 - DNA, 22 bp, synthetic construct
  source 1..22: mol_type = other DNA
caccatgtcgactttggaatct

SEQ ID NO: 68 - DNA, 18 bp, synthetic construct
  source 1..18: mol_type = other DNA
ttagacacatgccgccgt

SEQ ID NO: 69 - DNA, 23 bp, synthetic construct
  source 1..23: mol_type = other DNA
caccatggagctcctggacgtgg

SEQ ID NO: 70 - DNA, 19 bp, synthetic construct
  source 1..19: mol_type = other DNA
ctatactgacaagtgcagt

SEQ ID NO: 71 - DNA, 20 bp, synthetic construct
  source 1..20: mol_type = other DNA
atgacatacacgaaaactac

SEQ ID NO: 72 - DNA, 24 bp, synthetic construct
  source 1..24: mol_type = other DNA
gttagaggttttggctttcgacta

SEQ ID NO: 73 - DNA, 21 bp, synthetic construct
  source 1..21: mol_type = other DNA
cgaccgcacttgtaagcttag

SEQ ID NO: 74 - DNA, 21 bp, synthetic construct
  source 1..21: mol_type = other DNA
cgtcaacgtctacctcgtagc

SEQ ID NO: 75 - protein, 211 aa, synthetic construct
MDVIMLETQLDPFFLNHANFDFLNFISSLWTWVAVLAAAALSLWKIKSTVVVVVVVDSGSNNSQSSATTNKSDGGYVLLHQQQLQPAHEEKEEEINKLVRKSVTPSSCDSCVMYAQVNKVDGLTKNKFTOVYLEENVNDDGDDCTIVVDHEARNVEDINCNYNYIVRRVCSLESFEGKYVIRRNVQLGWYRYQQIAALSQSVVKLWDGERW

(19) United States
(12) Patent Application Publication
(10) Pub. No.: US 2024/0150785 A1
(43) Pub. Date: May 9, 2024

(54) CONFERRING DROUGHT TOLERANCE AND BIOMASS ACCUMULATION THROUGH THE PLANT-SPECIFIC RFS GENE FAMILY

(71) Applicants: Peter K. Lundquist, Okermos, MI (US); Sheng Ying, Lansing, MI (US); Wolf Scheible, Ardmore, OK (US)

(72) Inventors: Peter K. Lundquist, Okermos, MI (US); Sheng Ying, Lansing, MI (US); Wolf Scheible, Ardmore, OK (US)

(21) Appl. No.: 18/465,840

(22) Filed: Sep. 12, 2023

Related U.S. Application Data
(60) Provisional application No. 63/375,380, filed on Sep. 12, 2022.

(51) Int. Cl. C12N 15/82; C07K 14/415

(52) U.S. Cl. CPC C12N 15/8273; C07K 14/415; C12N 15/827; C12N 2830/002; C12N 2830/008

(57) ABSTRACT

Described herein are expression cassettes, plant cells, plant seeds, plants, and methods useful for delaying flowering, increasing biomass accumulation, and increasing drought tolerance in plants.

### CROSS REFERENCE TO RELATED APPLICATIONS

This application claims benefit of priority to the filing date of U.S. Provisional Application Ser. No. 63/375,380, filed Sep. 12, 2022, the contents of which are specifically incorporated herein by reference in their entirety.

### FEDERAL FUNDING

This invention was made with government support under 2021-67013-33756 awarded by the USDA/NIFA. The government has certain rights in the invention.

### INCORPORATION BY REFERENCE OF SEQUENCE LISTING

This application contains a Sequence Listing which has been submitted electronically in ST26 format and is hereby incorporated by reference in its entirety. Said ST26 file, created on Jan. 2, 2024, is named “3000211 US1.xml” and is 89,681 bytes in size.

### BACKGROUND

Terrestrial plants are exposed to a wide range of environmental stresses that severely affect their growth and development. Physical or chemical factors hostile to plant survival can include low or high temperature, deficient or excessive water, high salinity, heavy metals, and ultraviolet (UV) radiation. These abiotic stresses pose a severe threat to agriculture and the ecosystem, accounting for great crop yield loss. To ensure maximal reproductive success, plants have developed potent strategies to tolerate adverse abiotic stress. These strategies can have a substantial impact on the stress resilience of crops and ultimately on agricultural productivity.

Drought is one of the major limiting factors for plant productivity and spatial distribution. The annual loss in yield of major cereal crops due to drought is estimated to exceed ten billion dollars globally. Desertification, defined as “Land degradation in arid, semi-arid and dry sub-humid areas,” is happening in about 70% of the total of the world's dry lands (3.6 billion hectares) and has become a very distinctive global issue with major environmental consequences. It affects about 25% of the total land area of the world and about 17% of the world population. Conventional crop improvement for enhanced drought tolerance has been ineffective, mainly due to limited germplasm resources and incompatibility in crosses between distantly related plant species.

Development of drought-tolerant plant species through biotechnology is both economically and environmentally important. Recent advances in plant gene discovery and genetic transformation have begun to provide the tools for generating stress-tolerant crops using transgenic approaches. Despite the enormous economic and environmental significance, development of transgenic crops that confer drought tolerance in a highly controlled manner remains a challenge.

### SUMMARY

Described herein are plants, plant cells, and plant seeds that provide delayed flowering, increased biomass accumulation, and increased drought tolerance, as well as methods for making and using such plants, plant cells, and plant seeds. The nucleic acids, expression cassettes, plants, seeds, and methods described herein can be used to improve the quantity of plant materials for biofuel production and other uses. Methods of cultivating such plant seeds and plants are also described herein that include, for example, harvesting the plants, seeds, or the tissues of the plants.

For example, plant cells, plant seeds, and plants are described herein that include an expression system with at least one expression cassette comprising a first promoter operably linked to a nucleic acid segment encoding a Regulator of Flowering and Stress (RFS) polypeptide from the RFS family of genes. The expression system can further comprise at least one second expression cassette comprising a second promotor operably linked to a nucleic acid segment encoding a second RFS polypeptide.

In addition, methods are described herein that include growing a plant seed or plant having an expression system that includes the at least one first expression cassette comprising a first promoter operably linked to nucleic acid segment encoding an RFS polypeptide, to thereby produce a mature plant. Methods are described herein also include growing a plant seed or plant having an expression system that further includes the at least one second expression cassette comprising a second promoter operably linked to nucleic acid segment encoding an RFS polypeptide, to thereby produce a mature plant.

### BRIEF DESCRIPTION OF THE DRAWINGS

FIGS. 1A-H illustrates expression patterns of RFS genes in Arabidopsis thaliana. FIG. 1A is a graph showing relative expression of AtRFS1 in roots and shoots under P-sufficient and P-deficient conditions. FIG. 1B is a graph showing relative expression of AtRFS2 in roots and shoots under P-sufficient and P-deficient conditions. FIG. 1C is a graph showing the induction of AtRFS1 during P re-addition over a time course. FIG. 1D is a graph showing the induction of AtRFS2 during P re-addition over a time course. FIG. 1E is a graph showing relative expression of AtRFS1 and AtRFS2 in shoot or root under a deficiency in nitrogen (—N), sulfur (—S), or carbon (—C). FIG. 1F is a graph showing relative expression of AtRFS1 and AtRFS2 in different plant tissues. FIG. 1G is a graph showing expression changes of Medicago trunactula RFS genes under P-limitation. FIG. 1H is a graph showing expression changes of various monocot RFS genes under P-limitation.

FIGS. 2A-G illustrates expression patterns of RFS genes in Brachypodium distachyon. FIG. 2A is a graph showing relative expression of BdRFS in roots and shoots under P-sufficient and P-deficient conditions. FIG. 2B is a graph showing P-induction of AtRFS1 and AtRFS2 in a B. distachyon phr1 phi1 double mutant. FIG. 2C is a graph showing relative expression of BdRFS at different developmental stages of leaves. FIG. 2D is a graph showing relative expression of BdRFS under light (L), dark (D), and restored light (RL) growth conditions. FIG. 2E is a graph showing relative expression of BdRFS under drought conditions. FIG. 2F is a graph showing relative expression of BdRFS under freezing conditions. FIG. 2G is a graph showing relative expression of BdRFS after treatment with abscisic acid (ABA) and methyl jasmonate (MeJA).

FIGS. 3A-B illustrate expression of RFS genes under environmental stimuli. FIG. 3A a graph showing relative expression of BdRFS and BdBiP2 in B. distachyon with tunicamycin (Tm) treatment. The B. distachyon BdBiP2 gene is used as positive control of Tm treatment. FIG. 3B shows graphs demonstrating expression of A. thaliana RFS genes in response to freezing or ABA treatment and expression of changes in monocot RFS genes under drought, freezing, or ABA treatment.

FIG. 4 shows images of subcellular localization of GFP-tagged RFS proteins in N. benthamiana leaf epidermal cells. Green fluorescent protein (GFP)-fused constructs of AtRFS1/At3g43110 (Panels A to D) or BdRFS/Bd4g31140 (Panels E and F) are transiently expressed in tobacco leaves. After 48 hr post-infiltration, green fluorescence was imaged. Scale bar, 20 μm.

FIGS. 5A-C are images of phenotypes of wild-type and two BdRFS overexpressors (OX) of B. distachyon. FIG. 5A shows the wild-type and OX plants at approximately 35 days old. FIG. 5B shows the wild-type and OX plants at approximately 100 days old. FIG. 5C shows the wild-type and OX plants at approximately 135 days old.

FIGS. 6A-G Phenotypes of BdRFS overexpression lines. FIG. 6A is a graph showing RT-qPCR analysis of BdRFS (Bd4g31140) transcript levels in wild-type and OX lines. The BdUBC18 gene was used as reference housekeeping gene for normalization of RT-qPCR values. The data represent the mean values of three replicates ±1 SD. FIG. 6B is a graph showing flowering time of wild-type and OX lines (n=6). DAS, days after sowing. Overexpression of RFS is shown to delay flowering time by approximately forty days as compared to plants not overexpressing RFS. FIG. 6C is a graph showing the shoot dry weight of matured wild-type and BdRFS overexpressors (n=4). FIG. 6D is an image of representative phenotypes of wild-type and overexpressor lines under water-deficit stress for 2 weeks. FIG. 6E is an image of representative phenotypes of wild-type and overexpressor lines under water-deficit stress for 2 weeks that were then re-watered for 3 days. FIG. 6F is a graph showing leaf thickness of wild-type and overexpressor lines (n=21). FIG. 6G is a graph showing leaf relative water content (RWC %) of wild-type and overexpressors under control (well-watered) or water-deficit stress. The data represent the mean values of three replicates ±1 SD. Statistical significance of differences is tested by one-way ANOVA analysis (P<0.001) and is indicated by lower case letters.

FIGS. 7A-M illustrate the phenotypes of BdRFS CRISPR knock-out (ko) mutants. FIG. 7A is an image of representative phenotypes of wild-type (BD21-3) and two BdRFS CRISPR-ko mutants under long day growth conditions at approximately 20 days old. FIG. 7B is an image of representative phenotypes of wild-type (BD21-3) and two BdRFS CRISPR-ko mutants under long day growth conditions at approximately 50 days old. FIG. 7C is an image of representative phenotypes of wild-type (BD21-3) and two BdRFS CRISPR-ko mutants under long day growth conditions at approximately 110 days old. Arrows point to spikes. FIG. 7D is a graph showing flowering time of WT and CRISPR mutants (n=7). FIG. 7E is a graph showing shoot dry weight of matured wild-type and CRISPR-ko mutants (n=4). FIG. 7F is a graph showing flag leaf length (n=30) of matured wild-type and CRISPR-ko mutants. FIG. 7G is an image showing representative phenotype of wild-type and CRISPR-ko mutants after water-deprivation for 7 days. FIG. 7H is a graph showing leaf thickness (n=21). FIG. 7I is a graph showing leaf relative water content (RWC %) RWC (n=4 to 7) of matured wild-type and CRISPR-ko mutants. FIG. 7J is an image of DAB staining of leaves of wild-type and CRISPR-ko mutants. FIG. 7K is a graph showing quantification of malondialdehyde (MDA) contents in wild-type, CRISPR-ko mutants and OX2 (n=4). FIG. 7L shows images of representative TEM micrographs illustrating chloroplast ultrastructure of wild-type and CRISPR-ko leaves. Arrows point to plastoglobules (PGs). Scale bar, 2 μm. FIG. 7M is a graph showing the numbers of PGs per chloroplast area in wild-type and CRISPR-ko mutants. Measurements are collected from 10 micrographs of four different plants each, per genotype. All experiments are repeated three times and representative results are presented in the figure. Statistical significance of differences is tested by one-way ANOVA analysis (P<0.001) and is indicated by lower case letters.

FIGS. 8A-C illustrate the effects of loss of AtRFS1 and AtRFS2 on bolting time in A. thaliana. FIG. 8A is a representative image showing the bolting phenotype of atrfs1, atrfs2, and the corresponding double mutant in A. thaliana (“dmu”). Seedlings were germinated on W Murashige and Skoog (MS) solid medium for 7 days, then transferred to soil to grow until senescence. FIG. 8B is a graph showing bolting time of various genotypes. FIG. 8C is a graph showing total rosette leaves numbers of various genotypes at bolting. Statistical significance of differences is tested by one-way ANOVA analysis (P<0.001) and is indicated by lower case letters.

FIGS. 9A-O illustrate lipidomic analysis of BdRFS CRISPR-ko mutants and overexpression lines, and the A. thaliana RFS double mutant (dmu). FIG. 9A is a graph showing total lipid content in leaves of wild-type (BD21-3) and CRISPR-ko mutants grown under long day conditions. FIG. 9B is a graph showing lipid composition in leaves of wild-type (BD21-3) and CRISPR-ko mutants grown under long day conditions. FIG. 9C is a graph showing acyl group distributions of DGDG in leaves of wild-type (BD21-3) and CRISPR-ko mutants grown under long day conditions. FIG. 9D is a graph showing acyl group distributions of phosphatidylcholine (PC) in leaves of wild-type (BD21-3) and CRISPR-ko mutants grown under long day conditions. FIG. 9E is a graph showing a ratio of monogalactosyl diacylglycerol (MGDG) to digalactosyl diacylglycerol (DGDG) in leaves of wild-type (BD21-3) and CRISPR-ko mutants grown under long day conditions. FIG. 9F is a graph showing total lipid content in leaves of wild-type (BD21-3) and overexpressor lines grown under long day conditions. FIG. 9G is a graph showing lipid composition in leaves of wild-type (BD21-3) and overexpressor lines grown under long day conditions. FIG. 9H is a graph showing acyl group distributions of DGDG in leaves of wild-type (BD21-3) and overexpressor lines grown under long day conditions. FIG. 9I is a graph showing acyl group distributions of phosphatidylcholine (PC) in leaves of wild-type (BD21-3) and overexpressor lines grown under long day conditions. FIG. 9J is a graph showing a ratio of MGDG to DGDG in leaves of wild-type (BD21-3) and overexpressor lines grown under long day conditions. FIG. 9K is a graph showing total lipid content in shoots of A. thaliana wild-type (Col-0) and the AtRFS double mutant (dmu) grown in growth chamber. FIG. 9L is a graph showing lipid composition in shoots of A. thaliana wild-type (Col-0) and the AtRFS double mutant (dmu) grown in growth chamber. FIG. 9M is a graph showing acyl group distributions of DGDG in shoots of A. thaliana wild-type (Col-0) and the AtRFS double mutant (dmu) grown in growth chamber. FIG. 9N is a graph showing acyl group distributions of PC in shoots of A. thaliana wild-type (Col-0) and the AtRFS double mutant (dmu) grown in growth chamber. FIG. 9O is a graph showing ratio of MGDG to DGDG in shoots of A. thaliana wild-type (Col-0) and the AtRFS double mutant (dmu) grown in growth chamber. FIGS. 9A, 9F, and 9K: statistical significance of differences is tested by one-way ANOVA analysis (P<0.05) and is indicated by lower case letters. FIGS. 9B-D, 9G-I, and 9K-O: the data represent the molar ratio of total lipids and represent the mean values of three replicates ±1 SD. Acyl groups are designated with numbers of carbons:number of double bonds. *P<0.05, **P<0.01, and ***P<0.001 indicate statistical significance as determined by Student's t test. Ns, no significance.

FIGS. 10A-J illustrate a reduction of endogenous jasmonic acid (JA) and related metabolites in BdRFS CRISPR-ko mutants. FIG. 10A is a graph showing quantification of 12-oxophytodienoic acid (OPDA) in mature leaves of wild-type (BD21-3) and CRISPR-ko lines grown under long day conditions. FIG. 10B is a graph showing quantification of MeJA in mature leaves of wild-type (BD21-3) and CRISPR-ko lines grown under long day conditions. FIG. 10C is a graph showing quantification of JA in mature leaves of wild-type (BD21-3) and CRISPR-ko lines grown under long day conditions. FIG. 10D is a graph showing quantification of JA-Isoleucine (JA-Ile) in mature leaves of wild-type (BD21-3) and CRISPR-ko lines grown under long day conditions. FIG. 10E is a graph showing quantification of JA in mature leaves of wild-type (BD21-3) and overexpressor lines grown under long day conditions. FIG. 10F is a graph showing quantification of JA-Ile in mature leaves of wild-type (BD21-3) and overexpressor lines grown under long day conditions. FIG. 10G is a graph showing expression of JA-inducible gene, BdJAZ (Bd3g23190) in wild-type (BD21-3) and crispr #9, under MeJA treatment. FIG. 10H is a graph showing expression of JA-inducible gene, BdLOX (Bd1g11670) in wild-type (BD21-3) and crispr #9, under MeJA treatment. FIG. 10I is a graph showing expression of JA-inducible gene, BdMYC2 (Bd3g34200) in wild-type (BD21-3) and crispr #9, under MeJA treatment. FIG. 10J is a graph showing expression of JA-inducible gene, BdOPR3 (Bd3g37650) in wild-type (BD21-3) and crispr #9, under MeJA treatment. The data represent the mean values of three replicates ±1 SD. All experiments were repeated three times and representative results are presented in figures. Statistical significance of differences is tested by one-way ANOVA analysis (P<0.05) and is indicated by lower case letters.

FIG. 11 illustrates a model showing the role of RFS in drought tolerance and flowering time.

FIG. 12 illustrates an alignment of amino acid sequences of monocot and dicot RFS proteins (SEQ ID NOS: 1, 4, 7, 9, 11, 13, 15, 17, 19, 21, 23, 25, 27, 29, 31, 33, 35 and 75). Predicted transmembrane domain and conserved domains were respectively highlighted with black or purple color underlines. Two experimentally validated phosphorylation sites (Thr, T) in At3g43110 also underlined within that sequence.

FIGS. 13A-G show that BdRFS manipulates stomatal density and size. FIG. 13A and FIG. 13E are representative images showing the stomatal phenotype of BD21-3 (WT), BdRFS overexpression lines (OX2 and 3), and CRISPR/Cas9-mediated knockout mutants (CR3 and 9) under well-watered (FIG. 13A) and water deprived (FIG. 13E) conditions. The images are taken by a light microscope from fully expanded, mature flag leaves of different genotypes. Scale, 30 (FIG. 13A) or 20 (FIG. 13E) m. FIGS. 13B-D are box plots of stomatal density (n=12 individual plants of each genotype) (FIG. 13B), stomatal length (n=12 individual plants of each genotype) (FIG. 13C), and stomatal index (n=12 individual plants of each genotype) (FIG. 13D) in WT, OXs, and knockout mutants under well-watered condition. FIG. 13F and FIG. 13G are box plots of stomatal density (n=6 to 8 individual plants of each genotype) (FIG. 13F) and stomatal length (n=6 to 8 individual plants of each genotype) (FIG. 13G) in WT, OXs, and knockout mutants under water-deprived condition. FIG. 13C shows a well-watered condition (control); FIG. 13D shows a water deprived condition (drought stress). FIGS. 13B-D, F, and G: Horizontal line is the median and whiskers display minimum and maximum values. Statistical significance of differences was tested by one-way ANOVA and Tukey's analysis (p<0.01), and is indicated by lower case letters (B to D), or by Student's t-test and is indicated by asterisks (ns, no significance; *<0.05; **<0.01; ****<0.0001) (F and G).

FIGS. 14A-F illustrate biomass and yield-related characteristics of different genotypes grown under well-watered condition. Representative images of (FIG. 14A) 4-week-old and (FIG. 14B) 8-week-old OX2, CR9, and BD21-3 plants grown under well-watered condition. FIG. 14C shows fresh (left) and dry (right) biomass of 4-week-old plants (n=4 individual plants of each genotype). FIG. 14D shows water content (%) of 4-week-old plants (n=4 individual plants of each genotype). FIG. 14E shows dry biomass of each genotype at full maturity (n=7 to 8 individual plants of each genotype). FIG. 14F shows the harvest index (the ratio of grain to total above ground tissue dry matter) of each genotype (n=7 to 8 individual plants of each genotype). Statistical significance of differences was tested by one-way ANOVA and Tukey's analysis (p<0.01), and is indicated by lower case letters.

FIGS. 15A-C illustrate biomass and yield-related characteristics of different genotypes under water deprived condition. FIG. 15A depicts representative images showing the phenotypes of OX2. CR9, and WT after a 10-day water deprivation treatment. The arrow points at emerging spikes of OX2. FIG. 15B is a graph showing fresh (left) and dry (right) biomass. FIG. 15C is a graph showing water content (%) of the genotypes (n=6 individual plants). Statistical significance of differences was tested by one-way ANOVA and Tukey's analysis (p<0.01), and is indicated by lower case letters.

### DETAILED DESCRIPTION

Described herein are expression cassettes, plant cells, plant seeds, plants, and methods useful for delaying flowering time, increasing biomass accumulation, and promoting drought tolerance of plants. The plant cells, plant seeds, and plants express increased levels of Regulator of Flowering and Stress (RFS) family of genes. Such increase expression of RFS can be provided by incorporating one or more expression cassettes into the plant cells, plant seeds, and plants.

As disclosed herein and in the examples below, overexpression of a plant-specific gene family designated as Regulator of Flowering and Stress (RFS), a BdRFS gene in Brachypodium distachyon (B. distachyon), results in delayed flowering, increased biomass accumulation, and promotes drought tolerance. CRISPR/Cas9-mediated knockout mutants exhibited opposite phenotypes. A double T-DNA insertional mutant in two Arabidopsis thaliana (A. thaliana) homologs replicates the effects on flowering and water-deprivation seen in the B. distachyon CRISPR knockout lines, highlighting the functional conservation of the family between monocots and dicots. Lipid analysis shown below of B. distachyon and A. thaliana revealed that digalactosyldiacylglycerol and phosphatidylcholine contents were significantly, and reciprocally, altered in overexpressor and knockout mutants. Importantly, alteration of C16:0-containing phosphatidylcholine, a Flowering Locus T-interacting lipid, correlated with flowering phenotype, with elevated levels corresponding to earlier flowering. Co-immunoprecipitation analysis suggested that BdRFS interacts with Phospholipase D al as well as several other abscisic acid-related proteins. Furthermore, reduction of C18:3 fatty acids in digalactosyldiacylglycerol correlated with reduced jasmonic acid metabolites in CRISPR mutants. Collectively, stress-inducible RFS proteins represent a regulatory component of lipid metabolism that impacts several agronomic traits of biotechnological importance.

Plants have evolved elaborate mechanisms and complex regulatory networks to cope with environmental stresses. Such mechanisms often involve remodeling of membrane lipid composition (Liu et al., 2019). While chloroplast membranes are dominated by the galactolipids monogalactosyldiacylglycerol (MGDG) and digalactosyldiacylglycerol (DGDG), biological membranes outside of the chloroplast consist primarily phospholipids. Under phosphorus (P) deficiency, intracellular phospholipids are rapidly hydrolyzed by various phospholipases to conserve inorganic phosphate (Pi) for other essential processes, and their membrane structural roles are substituted by MGDG and DGDG, or the sulfolipid sulfoquinovosyldiacylglycerol (SQDG) (Nakamura, 2013; Pfaff et al., 2020; Sun et al., 2020; Ali et al., 2022). In P-starved A. thaliana, phospholipase D (PLD) ζ1 and ζ2 hydrolyze phosphatidylcholine to release Pi and diacylglycerol (DAG), promoting DGDG synthesis (Li et al., 2006). In addition, altering polyunsaturated fatty acid (PUFA) levels of membrane lipids can help plants adapt to environmental stresses through alteration of membrane fluidity and stability. For instance, overexpression of fatty acid desaturases (FADs) can enhance plant tolerance to numerous abiotic stresses (Yu et al., 2021).

Flowering time (also known as heading time in grasses) is a trait that determines grain yield, biomass production, and reproductive success. To maximize yield, the flowering time of cereals crops have been domesticated according to the specific requirements of the grower and crop species (Cockram et al., 2007). Delayed flowering is generally a desirable trait for biofuel or forage crops, where vegetative biomass is the material of use. In contrast, an early flowering trait is generally desired in cereal crops where a shortened time to harvest can permit additional plantings in a growing season and a reduced threat of biotic and abiotic stresses (Bheemanahalli et al., 2017; Shavrukov et al., 2017). The timing of flowering is regulated through complex systems, revealing a number of key floral integrators. Of particular note, Flowering Locus T (FT) plays a conserved and dominant role in determination of plant flowering time by regulating shoot meristematic activity (Jin et al., 2021). FT binds to phosphatidylcholine (PC), favoring species with more saturated acyl chains, to promote flowering (Nakamura et al., 2014). The acyl composition of PC oscillates with the diurnal cycle and changes in response to environmental stimuli. FT interacts most effectively with saturated PC species, such as C16:0, that accumulate during the light period. Thus, it was concluded that FT preferentially binds to more saturated PC species to promote flowering time.

On the other hand. FT can also interact with negatively charged phosphatidylglycerol (PG, especially 34:4 and 34:3 species) at low ambient temperature (Susila et al., 2021). This interaction inhibits the translocation of FT from companion cells to sieve elements, thereby preventing precocious flowering.

Flowering time is significantly affected by abiotic stresses such as drought or low temperature (Kazan and Lyons, 2015; Cho et al., 2017). During drought stress, plants flower earlier to escape the harsh environments, while nutrient starvation (e.g., N or P) results in delayed flowering (Kant et al., 2011; Riboni et al., 2013; Lin and Tsay, 2017). In B. distachyon, several key regulators of flowering time, such as FT1, FT-Like9 (FTL9), microRNA5200 and Early Flowering 3 (ELF3) have been identified and functionally characterized (Ream et al., 2013; Wu et al., 2013; Qin et al., 2017; Qin et al., 2019; Woods et al., 2019; Bouché et al., 2022). B. distachyon FT1 protein, and its closest paralog FT2, have been shown to regulate flowering time, spikelet development and fertility (Ream et al., 2013; Lv et al., 2014; Shaw et al., 2019). Ecological studies have shown that due to its low water use efficiency, B. distachyon prefers to live in higher, cooler, and wetter locations (i.e., north of 330 latitude) compared to other Brachypodium species (Scholthof et al., 2018). Nevertheless, how B. distachyon adjusts its habits (e.g., flowering time) to adverse environments is poorly understood.

As detailed herein, members of a conserved, plant-specific gene family were identified and named Regulator of Flowering and Stress (RFS) family. This gene family encodes proteins of approximately 22 kD, distinguished by a single N-terminal transmembrane helix and a conserved but uncharacterized domain. Transcript levels of several RFS genes were strongly affected by environmental stimuli (e.g., phosphorus availability, drought, or freezing stress). Overexpression of the B. distachyon homolog BdRFS (Bd4g31140) not only significantly delayed flowering/heading time, but also enhanced biomass accumulation and drought tolerance. Knockout mutants of RFS genes in A. thaliana or B. distachyon had early flowering phenotypes and were hypersensitive to water deficit. Thorough lipid analyses of mutants demonstrate that RFS proteins appear to modulate lipid metabolism to impact these agronomic traits. Thus, the RFS gene family is identified as a novel stress-inducible effector of flowering time and drought tolerance.

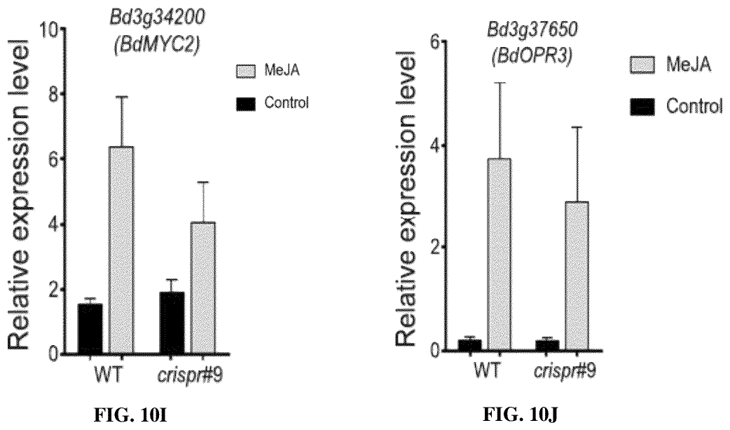

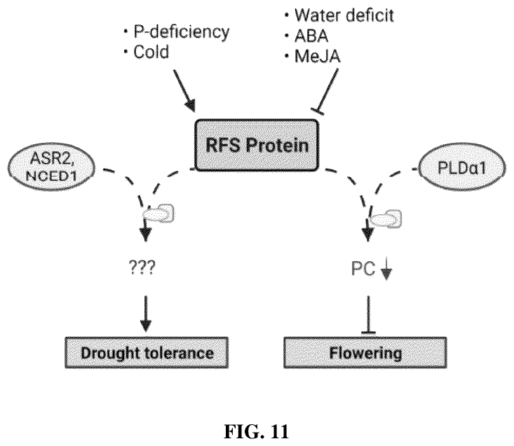

A model of the role of RFS in drought tolerance and flowering time is shown in FIG. 11. The RFS genes are induced by P-deficiency and cold stress and conversely are suppressed by water deficit, abscisic acid (ABA), or methyl jasmonate (MeJA) treatment. RFS induces polar lipid remodeling, including suppressed levels of phosphatidylcholine (PC) containing C16:0 which, in turn, inhibits flowering. The effect on the lipid composition is achieved through a direct interaction with Phospholipase D α1 (PLDα1). Furthermore, direct interactions of RFS with other abiotic stress-related proteins such as abscisic acid-, stress-, and ripening-related 2 (ASR2), or Nine-cis Epoxycarotenoid Cleavage Dioxygenase 1 (NCED1) work to promote drought tolerance through as yet unknown mechanisms. If such an effect operates through promotion of ABA biosynthesis/accumulation, then this could represent a feedback inhibition loop through the demonstrated downregulation of RFS transcript.

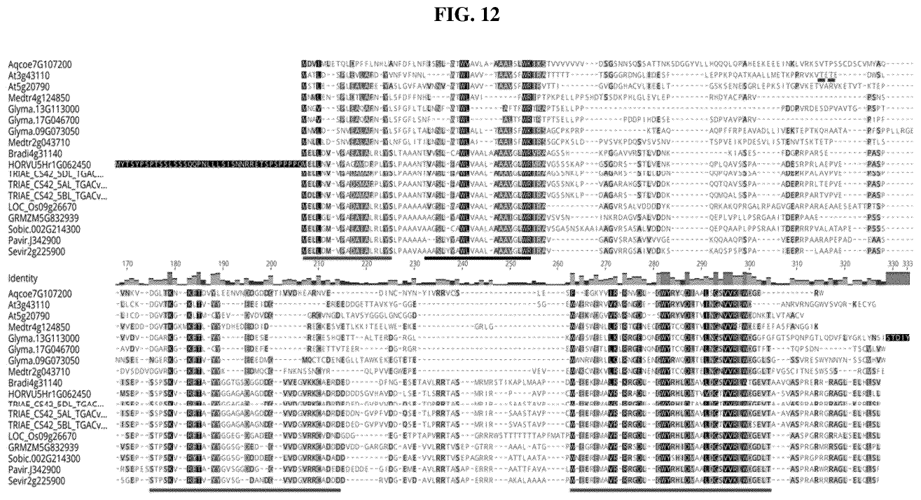

A variety of RFS genes can be used to improve drought tolerance and/or delay flowering time with concomitant increase in plant biomass. As shown in FIG. 12, an alignment of amino acid sequences of monocot and dicot RFS proteins illustrates the conservation of RFS genes in multiple plant species. For example, an amino acid sequence of a RFS protein from A. thaliana (At3g43110) is shown below as SEQ ID NO: 1.

MATLDSPLEV LAFDYVNFVF NNLWTWIAVV TAAVSFWRIR
ATTTTTTSGG GRDNGLIDES FLEPPKPQAT KAALLMETKP
PRVKVTETED WSLLLCKDGV TKGKLTVYYE EEIDGEREED
DGETTAVKYG GGESGEWWER WERVVKMRNG DEGWYRYVDL
TVINGNVVRL WDANRVRNGG WVSVQRKECY G

A nucleotide sequence that encodes the RFS protein (At3g43110) from A. thaliana is shown below as SEQ ID NO: 2.

GCATCTATGAAAGATCCACCGTTCAACTTGTCAAGATCCAATGGCTC
ACCATCTATCTCTCTTCAGATCCATCCAACGGCTAAGATTCAATCTC
TCCCCATACTAATCCTCTCTTTCTTTCTCCCTTTATATAACCACTGA
TTCAATCTTCTTCGTTAGGATATAATAAAAACATTTCAGAAATGGCG
ACTTTGGATTCTCCGTTAGAGGTTTTGGCTTTCGACTACGTTAACTT
CGTCTTTAACAATCTTTGGACATGGATCGCCGTCGTGACGGCCGCCG
TTAGTTTCTGGCGGATCCGAGCCACTACCACTACCACCACCAGCGGA
GGTGGTAGAGACAATGGCTTAATAGATGAATCTTTTCTTGAGCCACC
AAAACCACAAGCGACAAAGGCTGCTCTCCTTATGGAGACGAAACCTC
CTAGAGTCAAGGTAACGGAGACTGAGGATTGGAGTTTGTTGTTGTGT
AAGGACGGAGTAACGAAGGGGAAGCTAACCGTGTACTACGAAGAAGA
GATTGACGGAGAGAGAGAAGAAGATGACGGAGAGACAACGGCCGTTA
AGTATGGAGGAGGTGAGAGTGGAGAATGGTGGGAGAGATGGGAGAGA
GTGGTGAAGATGAGAAATGGAGATGAAGGTTGGTACCGTTACGTGGA
TTTAACGGTGATTAACGGAAATGTTGTGAGGTTGTGGGATGCTAACC
GTGTACGTAACGGTGGTTGGGTTAGTGTGCAACGTAAGGAGTGTTAT
GGTTGAGACATGTTGGGGACAAGTATAATTTTCTAATTTAAGCATAT
GATTATTCATTGAACCTTTTTGGTTTAAAGACTATTTTTGTTGAATT
AAAGTTGTAAT

A. thaliana has a second RFS gene (At5g20790) that encodes a first RFS protein and a second RFS protein. The nucleotide sequence that encodes the second RFS gene (At5g20790) is shown below as SEQ ID NO: 3.

ATTATACACACTTCAATAAAGAGATCGATGTCGACTTTGGAATCTCC
ATTAGAGGCGTTGGCGTTTGAATACGCTAGCTTAGGTGTTTTCGCCG
TCGTCAACAACGTCTGGACATGGATCGCCGTCGTGACTGCCGCTGTC
AGCTTCTGGAGGATCAGAGTCACAACCATAGGAGTCGGAGACGGCCA
TGCATGTGTCTTGATAGAAGAATTAACCGGTTCTAAATCTGAAAACG
AATCCGGTCGTCTCGAACCGAAATCAATAACCGGTCCGGTCAAAGAA
ACGGTTGCACGAGTGAAGGAAACGGTTACGAAAACGGAGCCGTTAAT
ATGCGATGACGGAGTGACAAAGGGGAAGCTGACGATGTGCTACGAGG
TAGACGTTGACGTTGACGGTGGGAGGTGTGTTAACGGAGATTTAACG
GCAGTTAGCTACGGAGGAGGTTTGGGTAATTGTGGCGGGGATTGGTG
GGAGAAATGGGATGGAGTGGTGAGGATGAGAAATGGTGATGACAGTT
GGTACCGTTACGTGGATTTAACGGTGATTAATGGAAATGTGGTAAGG
TTATGGGATGACAACAAAACACTAGTAACGGCGGCATGTGTCTAAAT
TAGAGAAGTTTCATATTTCGGAAAGTTTTTAAATCTTGAGAAGCTTT
CTTGGTTTGAAGTGTTTTTTTTTTGTTGGTTGATTAAGTTGTAATTT
GTAAATAATTTTCACACAAGAGACCAAGAAGGAACGCTT

The amino acid sequence of the first RFS protein encoded by the RFS gene (At5g20790) is shown below as SEQ ID NO: 4

MSTLESPLEA LAFEYASLGV FAVVNNVWTW IAVVTAAVSF
WRIRVTTIGV GDGHACVLIE ELTGSKSENE SGRLEPKSIT
GPVKETVARV KETVTKTEPL ICDDGVTKGK LTMCYEVDVD
VDGGRCVNGD LTAVSYGGGL GNCGGDWWEK WDGVVRMRNG
DDSWYRYVDL TVINGNVVRL WDDNKTLVTA ACV

The amino acid sequence of the second RFS protein encoded by the RFS gene (At5g20790) is shown below as SEQ ID NO: 5

MSTLESPLEA LAFEYASLGV FAVVNNVWTW IAVVTAAVSF
WRIRVTTIGV GDGHACVLIE ELTGSKSENE SGRLEPKSIT
GPVKETVARV KETVTKTEPL ICDDGVTKGK LTMCYEVDVD
VDGGRCVNGD LTACFFFVG

A RFS nucleotide sequence from Brachypodium distachyon (B. distacyon) (Bd4g31140) is shown below as SEQ ID NO: 6.

CCTCATCACACCAAACCCGCTTTGAAATTCTAATCCTATCTCGAACC
ACCGCCCCCAATCAATCACCGCCGCCGCGAATGGAGCTCCTGGACGT
GGTGCCGGCGGAAGCCATCGCTCTCCGCCTCTACTCCCTCACCGCGG
CGGCCAACACCGTCGTCTCGCTCTGCGCCTGGCTCGTCGCGGCCCTC
GCCGCCGCCGCCGTCGGCCTCTGGCGCGTCCGCGCGGCCGGCTCCTC
ACACAAACCCGGCGGCGCCGTAGTCCGAAGTACCCTCGTGGACAACA
AGAAGATAGCATCGGAATCCTTTGACGGACCACGGCCTGCTCGGTCC
GAGCCGGCGTCCCCAATTAGCGAGCCGAGCTCGCCGTCCAAGGTCCG
GTTCACGGCCTACTACGGCGGGACAGGATCTGACGGCGGCGACGATG
GAGTAGTGGAAGGCGTCAAGAAATGCGCGGAGAGGGACGAGGATGAT
TTCAACGGCGAGAGCGAGACGGCTGTGCTGAGACGGACGGCGTCGAT
GAGGATGAGGTCGACGATTAAGGCGCCCTTGATGGCGGCGCCGGACT
GGGAGGAGAAGGAGATGGCCCTCAGAAAGAGGGGCGATTTGGGTTGG
TACCGTCACCTCGACATGGCGGTGCTCGATGGCAGCGTCGTGAGGCT
CTGGACCGGCGAGGTCACCGCGGCGGTGCAGGCCTCGCCGAGGGAGC
GGCGGAGGGCAGGATTGGAACTGCACTTGTCAGTATAGAGACTGGAT
TATAATGTGTATATTGTGGTGTAGGATTACTGGATTGTGATTAGGGC
GGTATGACAATTTTGTCCTTTGGGCATCAGGGCATGTCATATCTTTG
AACTTTGAAGAGGGCCAAGGCTGAAATGAACAGCCCTCGCCCCAGGA
CATAAATGAAAACTGGCAATGCCAGTGTATTCTCTCCCAGTTTCTT

A RFS amino acid sequence encoded by the nucleotide sequence of SEQ ID NO:6 (Bd4g31140) is shown below as SEQ ID NO:7.

MELLDVVPAE AIALRLYSLT AAANTVVSLC AWLVAALAAA
AVGLWRVRAA GSSHKPGGAV VRSTLVDNKK IASESFDGPR
PARSEPASPI SEPSSPSKVR FTAYYGGTGS DGGDDGVVEG
VKKCAERDED DFNGESETAV LRRTASMRMR STIKAPLMAA
PDWEEKEMAL RKRGDLGWYR HLDMAVLDGS VVRLWTGEVT
AAVQASPRER RRAGLELHLS V

A RFS nucleotide sequence from Medicago truncatula (Medtr2g043710) is shown below as SEQ ID NO: 8.

CATCTTATTAGAGATCCCACCCACCCTCATTTCTCTCTTCCTCCCACTCCTTATATCTT
CAGCCTCCTCAATCCATTTCTCTTATTCAATTCATTCCTCTCAAACACACACACACCAC
AAAACTAAACCAAAACAATAAAGATAATGAACCTCTTGTTAGATTCCAACGTTGAGGCT
TTAGCCTTCAACTACTTAAACTTTGGTTTAATCACAATTCTCAACAACTTATGGACTTG
GCTTGCTATCACCGCCGCTCTCAGCTTCTGGAAGATTCGCTCATCCGGTTGTCCCAAAT
CACTAGACCCAGTTTCAGTAAAACCGGATCAGAGTGTCTCAGTTTCCAACGTTACATCA
CCGGTGGAAATAAAACCCGACGAGAGTGTCTTGATTTCCACTGAAAATGTAACGTCAAA
AACTGAGACACCACCAACATTATCAGATGTTTCTGATGATGTTGATGGTGTAAGGAAGG
GGAAGTTTACCTTGTATTACAAGGAGGACATGCAATGTGGATTCAACAAGAATAGTAGT
AATTGTTACCGGCAATTGCCGGTTGTTGAAGGGTGGGAACCGGAAGTTGAAGTTGAGTG
GTGGAAGTGTTGGGAGAAGGTGTTAAGGTTGAGAAATGGAGAGAATGAGAATGGGTGGT
ACACGTGTCAAGATTTAACGGAGCTTAATGGAAACGTGGTTAAAATTTGGGACGGTGGG
TTAACATTTGTTGGTAGTTGCATCACAAATGAATCATGGTCCAGTTCCAGATGTATGTC
TTTTGAATGATAATAATGAGAAAGGAAAAAAAAAAAAAGTAGAAATAGCTTTTTTTTTT
ATTAGGTGTAGTTAGGTTTATGTCGTGTTTGTTTGTGTTGGATTAATAATAATCTCTCC
ACTAAAGATTTGAGAGATTATTTAGATTTGAGAATGTATATATGGTAATTTGGTAGTGC
GGTTTGGAAATTTTGGATGATGAAATTGATTATTCTCAAATTTGAAATGAAATTGGCCA
AAAGCAACAAGCTTTGCGTATGTCTTTGAATCTTTCCTTCTATCTCATGTCATAATTTT
CTTTTTTAAGGTTTTCTACCTTATGTCATTATTATCTATTTTTTTTTATTTTTTTTATT
TGTTAGAAGAAAAAAGTTTG

A RFS amino acid sequence encoded by the nucleotide sequence of SEQ ID NO: 8 (Medtr2g043710) is shown below as SEQ ID NO: 9.

MNLLLDSNVE ALAFNYLNFG LITILNNLWT WLAITAALSF
WKIRSSGCPK SLDPVSVKPD QSVSVSNVTS PVEIKPDESV
LISTENVTSK TETPPTLSDV SDDVDGVRKG KFTLYYKEDM
QCGFNKNSSN CYRQLPVVEG WEPEVEVEWW KCWEKVLRLR
NGENENGWYT CQDLTELNGN VVKIWDGGLT FVGSCITNES
WSSSRCMSFE

A RFS nucleotide sequence from Medicago truncatula (Medtr4g124850) is shown below as SEQ ID NO: 10.

CCCATATTCCCATTGACGGTCCCCACTCACCTGTGTTATAGTTCTTCTCATATCACTCT
TTCTTCTCTTCTCTTCTCCATAAATAAATTCTTCTTTCACCACTCTCACTCACACCCCC
TCCACTCTTACCATCAAAAGTGAAAAGAAGAGAAGAACACGCTCACAGAGATGAACATG
TTAGAAAATTCTCCACTTGACACTCTAGCTTTCAACTACTTGAGCTTCGATTTCTTCAA
CCATTTATGGACATGGCTCGCCGTCATCTTCTGGAGGATCCCAACCCCTAAACCTGAAT
TGTTACCTCCTTCCCACGACACTTCCTCCGATAAACCTCACCTAGGTCTGGAAGTGTTG
GAACCTCGTCATGATTATGCTTGTCCAGCGCGTGTTCCTAGTAACAGCGTTGTTGAAGA
CGATGACGGAGTTACAAAGGGAAAGATGAAGTTTACCTTGTATTATTATGACCATGAAG
ATGAAGATGACATTGACAGAGAATGCAAGGAGAGTGTTGAGACGTTAAAAAAAATAACG
GAGGAATTATGGGAAGAGAAAGAAGGAAGATTAGGGTGGTGGGAGAGTTGGGAAAACTT
GTTGAGGACAAGAACTGGAGAGAACGAAGGTGGGTGGTATACATGCCAAGATTTAACGG
TGATTAACGGCAACGTTGTTAGGTTTTGGGATGAAGAGTTCGAGTTTGCTTCCTTTGCA
AATGGAGGAATCAAATAACAGCTTCAGCTGTATGTTCTTGTATGGTAGTAATCACTAAT
TAGATTAGATAGAGAAACATAAGTATGTTATAATAATATTGTTATCATCAATGTATATA
TGGTGGTGGGTTAATTAATTTGGATAATATATATCATGATAAGTTGATATATAATCATC
TCCTCATTCATGCTCTTTGGCTCTCAAATTTGTAACGCAAAGTGTTGATATAGAGCAAC
CAATTAATTCTCCGTACATTTTTTCTGTA

A RFS amino acid sequence encoded by the nucleotide sequence of SEQ ID NO: 10 (Medtr4g124850) is shown below as SEQ ID NO: 11.

MNMLENSPLD TLAFNYLSFD FFNHLWTWLA VIFWRIPTPK
PELLPPSHDT SSDKPHLGLE VLEPRHDYAC PARVPSNSVV
EDDDGVTKGK MKFTLYYYDH EDEDDIDREC KESVETLKKI
TEELWEEKEG RLGWWESWEN LLRTRTGENE GGWYTCQDLT
VINGNVVRFW DEEFEFASFA NGGIK

A RFS nucleotide sequence from Glycine max (Glyma.09G073050) is shown below as SEQ ID NO: 12.

TAGGTTCCGATTCTGGCATCTTTATTAGAGATCCCTACCTCACTTGTCGCTTCTCCCAC
CCTTTAAATCCTTTCCTTTCCTCTCACACAACACAAAAATTGTTAAATTCCCAATCCAA
TAGTTTTATTAGTTCATAAAAGGAAAAAGAAAATATGAATGTCTTGGATTCTAATGTGG
AAGCTCTAGCCTTCAACTACTTGAGCTTCGGTTTTCTCACTGCCCTCAACAACCTATGG
ACCTGGCTCGCTCTCTTAACCGCCGCCCTCAGCTTCTGGAAGATTCGCTCCGCGGGCTG
CCCCAAGCCCAGGCCCAAGACCGAGGCCCAAGCCCAACCTTTTTTCAGGCCTGAGGCTG
TCGCGGATTTGTTAATAGTGGAGAAAACGGAGCCGACGAAACAACATGCAGCGACGGCT
CCGTTTTCTCCACCGTTATTACGAGGAGAAAATAACAGTGAAGAAAACGGCGAGAGGAA
GGGGAAGTTTACCGTTTATTACGAGGAGGAGGACATGGGCATGCAATGCACGTGCGATG
AGAATGAGGGGTTGTTAACGGCGTGGAAAGAGAAAGAGGGGACCGAAACGGAGTGGTGG
AATAGGTGGGAGAGGTTGTTGCAGTTGAGAAATGGGGAGAGCGAGAACGGGTGGTACAG
GTGGCAGGACTTGACGGAGCTTAACGGCAGCGTCGTTAGGATATGGGACGGTGGGTTAA
GTGGTTCTTCCGTTAGGGAATCGTGGTACAACAACTACAACTCTAGCTGTATGCATGTC
TGGTAATGATAATAAGAATAGTGATGAAAAATAATAGTCCTTGTGTTGGATTTTAACTA
TTAAGCTTCTCTCCTTCAAGACTTTGGAGAGGTTGTTGAGATTTGAGAATTTGTATATA
TATGGTATTTTGGTAGTGGATATGCAATTTTTTATGAAGAAATTATGAAGTAGAGCAAT
AAATTAATTAAGCATCATCGTTGTTGTCGTTCACGCATTTGGGTTTTAATATTTGACAC
ACAAATGCTCGAAATCGAGCTTTGTTTATGTTTTTTCTTAGGCTTGTTTATGTTTTTTA
ATTTGTATTTATCGGTCATTATTGTTTCT

A RFS amino acid sequence encoded by the nucleotide sequence of SEQ ID NO: 12 (Glyma.09G073050) is shown below as SEQ ID NO: 13.

MNVLDSNVEA LAFNYLSFGF LTALNNLWTW LALLTAALSF
WKIRSAGCPK PRPKTEAQAQ PFFRPEAVAD LLIVEKTEPT
KQHAATAPFS PPLLRGENNS EENGERKGKF TVYYEEEDMG
MQCTCDENEG LLTAWKEKEG TETEWWNRWE RLLQLRNGES
ENGWYRWQDL TELNGSVVRI WDGGLSGSSV RESWYNNYNS
SCMHVW

A RFS nucleotide sequence from Glycine max (Glyma.13G113000) is shown below as SEQ ID NO: 14.

AATTGAATTGAGGAGGCATCTTAGGTTGTCGATTCGGAGCATCTTATGTGAGATCCCAT
ATTCTCCAACCATATAATAACCTTCACCACATTTCTCTCTCGCATTCAATTAATTCAAC
ATACACGTCATGAACGGTGTTTCTCCTCTAGAGGCCCAAGCCTTCAACTACCTGAGCTT
CGGTTTCCTCACCCTCCTCAACAATTTCTGGACATGGCTCGCCTTCACTTTCTGGAGGA
CCCGAGCCCCCACCTCCGAGTTGCTGCCACCACCCGATGACCCGGTTCGCGACGAGTCG
GATCCTGTTGCCGTGACCGGGCCAAGTCCAACTGTGGTTGACGTTGACGGTGCGAGGAA
GGGGAAATTTACCTTGTATTACGAGGATGAGTCTGAGAGGGAATGCGAGAGCCACCAGG
AGACGACGGCGCTAACGGAGAGAGACGGAAGAGGCCTGAAGTGGTGGGAGAGTTGGGAA
GAGTTGTTGAAAATAAGACGAGGAGAAGACCAGAACGGATGGTACACGTGCCAGGATTT
AACGGTGCTTAACGGCAGCGTTGTTAGGTTATGGGATGGTGGCTTCGGCTTTGGTACCT
CACCACAGAACCCAGGGACACTACAAGATGTGTTCTTGTATGGTAAATTATATAATAGC
ATATCTACCGATATTTATTAGCTTAAGACTGTTAGATTATATATTATTCAGAATATGTG
TATATGGTAATTTGCTAGTAGTACTTGTTTGGATGATGATAATTAATGATCATCGTGTA
TGATAATTTGAAACGCAAATGATCAAAGCAATCTTAATTTGCGTACTTTTTTTTTT

A RFS amino acid sequence encoded by the nucleotide sequence of SEQ ID NO: 14 (Glyma.13G113000) is shown below as SEQ ID NO: 15.

MNGVSPLEAQ AFNYLSFGFL TLLNNFWTWL AFTFWRTRAP
TSELLPPPDD PVRDESDPVA VTGPSPTVVD VDGARKGKFT
LYYEDESERE CESHQETTAL TERDGRGLKW WESWEELLKI
RRGEDQNGWY TCQDLIVING SVVRLWDGGF GFGTSPQNPG
TLQDVFLYGK LYNSISTDIY

A RFS nucleotide sequence from Glycine max (Glyma.17G046700) is shown below as SEQ ID NO: 16.

GTGGAATGAAATGGGATGTAGTTGAATTGAAGAGGCATCTTAGGTGGGCGATTCGGAGC
ATCTTATGTGAGATCCCATATTCTTCAACCTTACTATAATAATAACCTTCACCACATTT
CTCTCGCGTATTCAACATCATGAACGCTGTTTCTCCTCTAGAGGCCCAAGCCTTCAACT
ACTTGAGCTTCGGCTTCCTCAGCAATTTCTGGACCTGGCTCGCCCTCACTTTCTGGAGG
ACCCGAACTCCCACCTCCGAGTTGCTCCCACCACCCGACGACCCGATTCACGACGAGTC
GGAGTCGGACCCTGTTGCCGTACCGGCGCGTGTTCCAATTCCATTTGCCGTTGACGTTG
ACGGAGCCAGGAAGGGGAAGTTTACCTTGTGTTACGAGGATGAGTTTGAGAGGGAATGC
GAGACGACGGTAACAGAGGAGAGAGACGGAAGAGGCCGGGAGTGGTGGGAGAGTTGGGA
AACCTTGTTGAAACTGAGACGAGGAGAGAACCAGAATGGCTGGTACACTTGCCAGGATT
TAACGGCGCTTAACGGCAGCGTTGTTAGGTTATGGGATGGTACCTTCACCACACAACCC
AGTGACAACACAAGCTGTGTTCTTGTATGGTAAAATTTGGTAATACTATAGCTAGCAGA
AATAATATTAGCTTAGATTGTTTCCACTGTTGGATTACTATTGAGAGAATGTATATATG
GTAGTAATTTGCTACTAGTCGTTGTTTGGATAATGATCATCTTGTATGGTACTAATTTC
AATC

A RFS amino acid sequence encoded by the nucleotide sequence of SEQ ID NO: 16 (Glyma.17G046700) is shown below as SEQ ID NO: 17.

MNAVSPLEAQ AFNYLSFGFL SNFWTWLALT FWRTRTPTSE
LLPPPDDPIH DESESDPVAV PARVPIPFAV DVDGARKGKF
TLCYEDEFER ECETTVTEER DGRGREWWES WETLLKLRRG
ENQNGWYTCQ DLTALNGSVV RLWDGTFTTQ PSDNTSCVLV
W

A RFS nucleotide sequence from Zea mays (GRMZM5G832939) is shown below as SEQ ID NO: 18.

ATGGAGCTCCTCGGCCTGGTGCCCGCGGAGGCCATAGCGCTCCGCCTCTACTCCCTCCC
CGCAGCAGCCGCGGCCGCCGGCTCGCTCTACGCCTGGCTCGTCGCCGCACTCGCCGCCG
CCATCGGCCTCTGGCGCATCCGCGCCGTCAGCGTTTCCAACATCAACAAGCGTGACGCA
GGCGTCAGCGCCCTCGTCGACGATAACCAGGAGCCACTGGTGCCTCTTCTGCCGTCTCG
GGGGGCTGCCATTACTGACGAGCCGCCCGCCGCCGAGCCGTCTTCCGTGAGCGAGCCGA
GCACGCCGTCCAAGGTTCGGTTCACTGCGTACTACGGTGGCTCGGGAGACGAAGACGGA
GTGGTCGACGGCGTCAGGAAATGCGCGGATGACGACGACGTCGACGACGGTGCGCGCGG
CCGAGACTGCGCGGTGGAGGTGGTTCTCAGAAGGACCGTGTCAGAGCCGGGGACAAGAA
GAGCGGCGGCCCTGGCCACGGGGCCGTGGGAAGGGAGGGAGATGGCGGTGAGGCGGAGG
AGTGACCTGGGTTGGTACCGCCACATCGACATGGCCGCGCTCGACGGCAGCGTCGTGAG
GCTGTGGGACGGCGACCTGACGGCGTCGCCGAGGGGGGGGATGAGGAGGTCAGGATTGG
AATTGCAATTGCCATTATAG

A RFS amino acid sequence encoded by the nucleotide sequence of SEQ ID NO: 18 (GRMZM5G832939) is shown below as SEQ ID NO: 19.

MELLGLVPAE AIALRLYSLP AAAAAAGSLY AWLVAALAAA
IGLWRIRAVS VSNINKRDAG VSALVDDNQE PLVPLLPSRG
AAITDEPPAA EPSSVSEPST PSKVRFTAYY GGSGDEDGVV
DGVRKCADDD DVDDGARGRD CAVEVVLRRT VSEPGTRRAA
ALATGPWEGR EMAVRRRSDL GWYRHIDMAA LDGSVVRLWD
GDLTASPRGR MRRSGLELQL PL

A RFS nucleotide sequence from Hordeum vulgare (HORVU5Hr1G062450) is shown below as SEQ ID NO: 20.

GTGTATCAGTGCACTTGTTTACCTAATCCAACATTCACTAAATATATAGAAATTGCAAA
AGCTCGCAGTACTAAACTGCAAATTCCATCATTTCCATTCCGCCACTTATATGTACACT
TCCTACCCAAGCCCAACCTCCTCACTATCCTCATCGCAACAACCAAACTTGCTCCTTTC
TATCTCGAACAACCGCCGAGAGACCTCCCCGTCGCCGCCGCCGCCGCAAATGGAGCTGC
TCAACGTGGTGCCGGCGGGCGCCATGGACTTCCCCCTCTACTCCCTCCCCGCGGGGGCC
AACACCGTCGCCTCGCTCTTCGCCTGGCTCGTCGCGGCACTCGCCGCCGCCGTCGGCCT
CTGGCGCATGCGCGCGGTCGGCTCCTCCAGCAAAGCACCCGGCTCTGGCGACCGCAGCA
GCACCCTCGTGGAAAACAAGCAGCCGCTGCAGGCTGTTTCCTCGCCTGCCGCTGACGAG
TCACGACCCAGACTCGCCGAGCCGGTGGAGCCGGCTTCCCCGATGAGCGAGCCGAGCTC
GCCGTCCAAGGTCCGGTTCACGGCGTACTACGGCGGGGCCGGAGCTGACGCCGGCGATG
ACGGAGTAGTGGATGGCGTCAGGAAATGCGCGGACAGGGACGATGACGACGACAGCGGT
GTGCATGCCGTCGATGACCTGAGCGAGACGCTGTCGAGACGGACGGCGTCGATGAGGAT
CAGGCCGGCGGTGCCCTGCTGGGAGGAGAGGGAGATGGCCGTGAGGAGGCGGGCAGATC
TGGGCTGGTACCGCCACCTTGACATGACGGTGGTCGACGGCACCGTTGTAAGACTGTGG
GACGGCGAGGTCACCGCGTCGGTGGCGTCGCCGAGGGCGCGGCGGGGGAGGGCAGGATT
GGAACTGCGCCTGTTACTATAGAGAAGGAGATCCTAATGTGTGTATAATGTGGTTAGGA
TTATTGGAATGTTATTATGGTGGGTATGACAATTTTGTCCTTCAATTTGGGCATGTCAT
GTGTATGTGTATCATTGAAAGAGGGCCGAGGCTGAATAAATGAACAGCCTTCGTCTCAG
CTCTCAGGAGAAATGAAAATTTGGCAAATGGCAGAGTATTCTTTGCTCAGTTCTCTGCT
TCCA

A RFS amino acid sequence encoded by the nucleotide sequence of SEQ ID NO: 20 (HORVU5Hr1G062450) is shown below as SEQ ID NO: 21.

MYTSYPSPTS SLSSSQQPNL LLSISNNRRE TSPSPPPPQM
ELLNVVPAGA MDFPLYSLPA AANTVASLFA WLVAALAAAV
GLWRMRAVGS SSKAPGSGDR SSTLVENKQP LQAVSSPAAD
ESRPRLAEPV EPASPMSEPS SPSKVRFTAY YGGAGADAGD
DGVVDGVRKC ADRDDDDDSG VHAVDDLSET LSRRTASMRI
RPAVPCWEER EMAVRRRADL GWYRHLDMTV VDGTVVRLWD
GEVTASVASP RARRGRAGLE LRLLL

A RFS nucleotide sequence from Oryza sativa (LOC_Os09g26670) is shown below as SEQ ID NO: 22.

ATGGAGCTCCTCGACATGGTGCCCGCGGACGCCATCGCGCTCCGCCTCTACTCCCTCCC
GGCGGCGGCGGCGGCCGTCGGCTCGCTCTGGGCGTGGCTCGTCGCCGCCCTCGCCGCCG
CCGTCGGCCTGTGGCGCATCCGCGCCGCCGCCGGTGTCCGTAGCGCCCTCGTGGACGAC
GACGACTACAAGCAGCGCAAGGCGAAGCAGCCGCGCGGTGCTCTGCGGCCTGCCGGCGT
CGGCGAGGCACGGCCGGCTCGCGCCGAGGCGGCGGAGTCGGAGGCGACGACGCCGACCT
CCCCGAGCGAGCCGAGCACGCCTTCCAAGGTCCGGTTCACGGCGTACTACGGCGGCGAG
GGGGACGGCGCCGACGAGGGAGTCGTGGACAGCGTCAGGAGATGCGTGGACAACGACGG
CGACGGCGAGGGGGAGACGCCGACGGCGCCGGTGAGGCGGACGGCGTCGGGGAGGAGGA
GGTGGTCGACGACGACGACGACGACGACGGCGCCATTCATGGCGACGCCGTGGGAGGAG
AGGGAGATGGCCGTGAGGAGGAGGGGAGACCTGGGGTGGTACCGCCACCTCGACATGGC
GGCGCTCGACGGAAGCGTCGTCCGGCTGTGGGACGGCGAGGTCACGGCGGCGTCGCCGG
GGCGGCGCGGCCGGAGGGCATTATCGGAATTGCACCTCTCATTGTAG

A RFS amino acid sequence encoded by the nucleotide sequence of SEQ ID NO: 22 (LOC_Os09g26670) is shown below as SEQ ID NO: 23.

MELLDMVPAD AIALRLYSLP AAAAAVGSLW AWLVAALAAA
VGLWRIRAAA GVRSALVDDD DYKQRKAKQP RGALRPAGVG
EARPARAEAA ESEATTPTSP SEPSTPSKVR FTAYYGGEGD
GADEGVVDSV RRCVDNDGDG EGETPTAPVR RTASGRRRWS
TTTTTTTAPF MATPWEEREM AVRRRGDLGW YRHLDMAALD
GSVVRLWDGE VTAASPGRRG RRALSELHLS L

A RFS nucleotide sequence from Panicum virgatum (Pavir.2KG347804) is shown below as SEQ ID NO: 24.

CCTCCTCCTCTCTCTCTCTCTCTCTCTCTCTCTCTCTCTCTCTCTCTCT
CTCTCTCTCTCTCTCCCCAGTCTCCAACAACCGCCCCGCCGCGAATGGA
GCTCCTCGACATGGTGCCCGCGGACGCCATCGCGCTCCGCCTCTACTCC
CTCCCGGCAGCGGCGGCGGCCTCGCTCTACTACGCCTGGCTCGTCGCCG
CGCTCGCCGCCGCCGTCGGCCTGTGGCGCATCCGCGCGGTCGGCGCCGG
CGTCAGCAGAGCCAGCGCCGTCGTCGTCGTCGGAGAAAAGCAGCAGGCG
GCCCAGCCGTCGCCCGCCGTTGAGGAGCCGCGGCCGGCGGCGCGGGCGC
CCGAGCCGGCTGATGAGGCGGCGTCCCGGAGCGAGCCAGAGTCGAGCAC
GCCGTCCAAGGTCCGGTTCACGGCGTACTACGGTGTTTCCGGAGGCGAC
GACGGCGGAGTGGTGGACAGTGTCAGGAGATGCGCGGACGAGGACGAGG
ACGAGGACGACGAGGGGATCGATGGCGAGATGGAGGCGGTCCTGAGGAG
GACGGCGTCGGCGCCGGAGAGAAGAAGGGCGGCGACGACCTTCGCGGTG
GCACTGTGGGAGGAGAGGGAGATGGCCGTGAGGCGGAGGGGCGACCTGG
GGTGGTACCGGCACCTCGACATGGCGGCGCTCGACGGCAGCGTCGTGAG
GCTGTGGGACGGCGAGCTGACGGCGTCGCCGAGAGCGCGGTGGAGGAGG
GCAGGATTGGAATTACAGTTGTCATTTTAGAGATGGTGTATGCTTAATC
TGTATATTGTGGTTGTATTGTAGGAGTAATGGGATGATGTTATTAGGAG
GATATGGCAATTTTGCCCTTTGAGATTGTCATCAAGTTATGGCCAAGGC
CAAAGGGCAAAGAAGGTCCCTTTGTCACTAGTCCTGTACCCGAGCCAGG
GCACAAATGGAAACTGATAAATGTCAGTGGTATCTATCATTCTACCTCC
GACCTCCCTCCTACCTCAGGCCCTTTGCATCCATTGTGTCTTCTACCAT
TCTGCCTTTT

A RFS amino acid sequence encoded by the nucleotide sequence of SEQ ID NO: 24 (Pavir.2KG347804) is shown below as SEQ ID NO: 25.

MELLDMVPAD AIALRLYSLP AAAAASLYYA WLVAALAAAV
GLWRIRAVGA GVSRASAVVV VGEKQQAAQP SPAVEEPRPA
ARAPEPADEA ASRSEPESST PSKVRFTAYY GVSGGDDGGV
VDSVRRCADE DEDEDDEGID GEMEAVLRRT ASAPERRRAA
TTFAVALWEE REMAVRRRGD LGWYRHLDMA ALDGSVVRLW
DGELTASPRA RWRRAGLELQ LSF

A RFS nucleotide sequence from Setaria viridis (Sevir.2G225900) is shown below as SEQ ID NO: 26.

TCCATTCCTCCACACCGCACCAGGGTCATCCCCCACCTCCTCTCTCGCT
CTCCAACCACCGCCGAGACCTCCCCACCACCACTTACCATCGAAACGCC
GCGAATGGAGCTCCTCGACATGGTGCCCGCGGACGCCATCGCGCTGCGC
CTCTACTCCCTCCCCGCAGCGGTAGCCGCCGCCGCCTCGCTGTACTACG
CCTGGCTCGTCGCCGCGCTCGCTGCCGCCGTAGGTCTCTGGCGCATCCG
CGCGGTCAGCGCCGGCGTTCGCAGAGGCAGCGCCATCGTCGATGACAAG
TCGAAGGCCCAGTCTCCTTCGCCGTCTCCTGCCATCGAAGAGCCGCGGC
TGGCGGCTCCGGCCGAGCCGGCGTCCCCGAGCGGCGAGCCGAGCACGCC
GTCCAAGGTCCGGTTCACGGTGTACTACGGTGTTTCGGGAGACGCCAAC
GACGGAGTTGTGGACGGCGTTCGGAGATGCGCGGACGACGACGACAGGG
TCGACGGCGAGGTAGACGCGGTCCTGCGAAGGACGGCGTCGGCGCCGGA
GAGAAGAAGGGCGAAGGCCCTGGCGGCGGCGGCGCCGTGGGAGGAGAGG
GAGATGGCCGTGAGGCGGAGGGGCGACCTGGGGTGGTACCGCCACCTCG
ACATGGCGGCGCTCGACGGCAGCGTCGTGAAGCTGTGGGACGGCGAGCT
GACGGCCTCGCCGAGAGCGCGGAGGAGGAGGGCAGGATTGGAATTACAG
TTGTCATTTTAGAGATGGCATATGGTTAATCTGTATATTGTTGTTGTAG
GAGTAATGGAATGATGTTATTAGGTGGATATGGCAATTTTGCCCTTTGA
TATTGTCGTAAGTTATGGCCAAGGCCAAAGGGCGAAGATGGGCTCTTTG
TGTGCGATGGCACAATTGGAAACTGATAAATGTCAGTGGTATCCTAGCT
CAGATCCTTGCATCCATGGTGTCTAGCTGCTGCTGCTGCTGCTTCTTCT
TCTTTTATTTTGATTTTCTTGCATGTTTACATTAGAAGTGGTGCTTCAG
AACGCATTTAGTTTTATCGGTTGTACTATTTTTTTTTACTGACCTATCA
ATTCCAGTTCCAGTATGAAGGGAAA

A RFS amino acid sequence encoded by the nucleotide sequence of SEQ ID NO: 26 (Sevir.2G225900) is shown below as SEQ ID NO: 27.

MELLDMVPAD AIALRLYSLP AAVAAAASLY YAWLVAALAA
AVGLWRIRAV SAGVRRGSAI VDDKSKAQSP SPSPAIEEPR
LAAPAEPASP SGEPSTPSKV RFTVYYGVSG DANDGVVDGV
RRCADDDDRV DGEVDAVLRR TASAPERRRA KALAAAAPWE
EREMAVRRRG DLGWYRHLDM AALDGSVVKL WDGELTASPR
ARRRRAGLEL QLSF

A RFS nucleotide sequence from Sorghum bicolor (Sobic.002G214300) is shown below as SEQ ID NO: 28.

CGCCTGTTCAATTTCAGTTCGGTAAATTGGTATTACACAGTCTGAGCGA
TACTCTGCCTCTGTTCCAATATACCGTACAAATTAAAGATGGATGGAAT
AGGTGCACTAGCTCAACTCCACAAAAGACTCAAATCATATACGAAATTG
TTCATCTAAAAATTGGAGGCTGAATGATTGCAAAATGCAGCTTATACTG
CACCCAAGAAAGAAAATTGGAAAATGCTCAGTTGTGCAGGTGAAAGCCT
GCAATAATTCCAGGCAGTTCACCAGTGCACATTCGTTCCCACTCCCGCC
ACCTATATATACGCTTCCCACCCAAACCCATCGTCTTCCATTCCTCACC
GCACACCAACACCATCCCCCTCTTCAACAACCGCCGAGACCTCCCCCAC
CAACACCACCACAACCACAACCACACCGCCGCGAATGGAGCTCCTCGGC
ATGGTGCCCGCGGAGGCCATTGCGCTGCGCCTCTACTCCCTCCCCGCGG
CGGCCGTGGCCGCGGGCTCGCTCTGCGCCTGGCTCGTCGCCGCGCTCGC
CGCCGCCGCGGTCGGCCTCTGGCGCATCCGCGCCGTCAGCGGCGCTTCC
AACTCCAAGGCCGCCATCGCCGCCGGTGTTCGCAGCGCCAGCGCCCTCG
TCGACGACGATAATCAGGAGCCACAGGCGGCGCCTCTGCCTCCGTCTCG
GGCTGCCATTACTGACGAGCCGCGGCGGCCGGTCGCTCTCGCTACGGCG
CCCGAGCCGTCGTCGTCCGTGAGCGAGCCGAGCACCCCATCGAAGGTTC
GGTACACTGCGTACTACGGCGGCTCAGGAGACGACGACGACGAAGGATT
GGTGGACGGCGTCAGGAAATGCGCGGACGCGGACGACGACGGCGAGGCG
GAGGTGGTTCTCAGAAGAACGGTGTCAGAGCCGGCGGGGAGAAGAAGAG
CGACGACCTTGGCGGCCACGGCGCCGTGGAATGAAGAGAGGGAGATGGC
GGTGAGGCGGAGGAGCGACCTGGGTTGGTACTCCCACATCGACATGGCA
GCGCTGGACGGCAGCGTCGTCAGGCTGTGGGACGGCGACCTGACGGCGT
CGCCGAGAGGGCCGCGGAGGAGGGCAGGATTGGAATTGCAACTCTCACT
GTAAGAGAGGTAGTGTGGTTAATGCGGGCATGTAATGTATATGTGGTAG
GGACAATGGGTTGTTAGTAGGAGGATATGACGATTTTGGGCATAAGATT
GTCATAAGTTATAGCCAGGGCAAAGAGCTCTTCTTCAATCGTGTGCTTG
AATATGGTATGGTTCCTCGCTGCTGGTCTGGAAAAGATTAGACTTACGA
GAACACAGTTTAAAGTTTAGCCTCTTTGGAGCATGTTCATTTGTCCGAA
ATGACATGACTAAAAGTATTGTTCGCTGATTTATTATATAAGAGAAAAA
CACTCTTGG

A RFS amino acid sequence encoded by the nucleotide sequence of SEQ ID NO: 28 (Sobic.002G214300) is shown below as SEQ ID NO: 29.

MELLGMVPAE AIALRLYSLP AAAVAAGSLC AWLVAALAAA
AVGLWRIRAV SGASNSKAAI AAGVRSASAL VDDDNQEPQA
APLPPSRAAI TDEPRRPVAL ATAPEPSSSV SEPSTPSKVR
YTAYYGGSGD DDDEGLVDGV RKCADADDDG EAEVVLRRTV
SEPAGRRRAT TLAATAPWNE EREMAVRRRS DLGWYSHIDM
AALDGSVVRL WDGDLTASPR GPRRRAGLEL QLSL

A RFS nucleotide sequence from Triticum aestivum (TRIAE_CS42_5AL_TGACv1_376348_AA1235560) is shown below as SEQ ID NO: 30.

CCCTCCTCACTATCCTCATCATTCACAACAAACTCACAGACCAAGCCTCCTCCTTCCTATCT
TGAACAACCGCCGGGAGGCCTCCCCGTCGCCGTCGCCGCAAATGGAGCTGCTCAACGTGGTG
CCGGCGGACTCCATGGCCTTCCCCCTCTACTCCCTCCCCGCGGCGGCCAACACCGTCGCCTC
GCTCTTCGCCTGGCTCGTCGCCGCCCTCGCCGCCGCCGTTGGCCTCTGGCGCATCCGCGCGG
TCGGCTCCTCCAACAAACTACCCGTCGCCGGCGCCCGCGCCCACGGCAGCACCCTCGTGGAC
GACAAGCAGCAGACGCAGGCCGTTTCGTCGCCTGCCGCTGACGAGCCACGGCCCAGACTCAC
CGAGCCGGTGGAGCCGGCTTCCCCGATTAGCGAGCCGAGCTCGCCGTCCAAGGTCCGGTTCA
CGGCGTACTACGGCGGGGCCGGAGCTGACGCCGGCGACGACGGAGTAGTTGACGGCGTCAGG
AAATGCGCCGACAGGGACGAGGACGACAACGGCGTGCCCTTCGTCGACGACCAGAGCGAGAC
GCTGCCGAGACGGACGGCGTCGATGAGGATCAGGTCGGCGGCCTCGACGGCGGTGCCCTGCT
GGGAGGAGAGGGAGATGGCCGTGAGGAGGCGGGGGGATCTGGGCTGGTACCGCCACCTTGAC
ATGGCGGTGCTCGACGGCACCGTCGTAAGGCTGTGGGACGGCGAGGTCACCGCGGCGGTGGC
GTCGCCGAGGGCGCGGCGGGGGAGGGCAGGATTGGAACTGCACCTGTCACTATAGAGATGGA
GATCCTGATGTGTATAATGTGGTTAGGATTGTTGGACTGTGATTAGGTGGGTATGACAGTTT
TGTCCTTCAATTTGGGCATGTCATATGTATGTGATGTGTATCATGGAAGAGGGCCGAGGCTG
AAAAATGAACAGCCTTCGTCTCAGCTCTCAGGAGAAACGAAAATTTGGCAAATAATGGCAGA
GTATTCTTGCTCAGTTCCCTTCCCTGCTTCCATTTCCATGGCTTGCACTGCTCTCCATTACA
TCCTTCTGACCCTTTTGTTCTCTCGG

A RFS amino acid sequence encoded by the nucleotide sequence of SEQ ID NO: 30 (TRIAE_CS42_5AL_TGACv1_376348_AA1235500) is shown below as SEQ ID NO: 31.

MELLNVVPAD SMAFPLYSLP AAANTVASLF AWLVAALAAA
VGLWRIRAVG SSNKLPVAGA RAHGSTLVDD KQQTQAVSSP
AADEPRPRLT EPVEPASPIS EPSSPSKVRF TAYYGGAGAD
AGDDGVVDGV RKCADRDEDD NGVPFVDDQS ETLPRRTASM
RIRSAASTAV PCWEEREMAV RRRGDLGWYR HLDMAVLDGT
VVRLWDGEVT AAVASPRARR GRAGLELHLS L

A RFS nucleotide sequence from Triticum aestivum (TRIAE_CS42_5BL_TGACv1_407440_AA1356920) is shown below as SEQ ID NO: 32.

CTCCTCACTATCCTCATCATTCACAACAAACCCAAAAACCAAACCTGCTCCTTCCCTAT
CTTGAACAACCGCCCGGAGACCTCCCCGTCGCCGTCGCCGTCACCGCAAATGGATCTGC
TCAATGTGGTGCCGGCGGACGCCATGGCCTTCCCCCTCTACTCCCTCCCCGCGGCGGCC
AACACCGTCGCCTCGCTCTTCGCCTGGCTCGTCGCGGCCCTCGCCGCCGCCGTCGGCCT
CTGGCGCATCCGCGCGGTCGGCTCCTCCAACAAACTACCCGACGCCGGCGCCCGCAGCA
GCACCCTCGTGGACGACAAGCAGCAGATGCAGGCTCTTTCGTCGCCTGCCGCTGACGAG
CCACGACCCGCACGCACCGTGCCGGTGGAGCCGGCTTCCCCTATTAGCGAGCCGAGCTC
GCCGTCCAAGGTCCGGTTCACGGCGTACTACGGCGGGGCCGGAGCTGACGCCGGCAACG
ATGGAGTAGTGGATGGCGTCAGGAAATGCGCGGACAGGGACGAGGACGAGGACGGCGTG
CCCGTCGTCGACGACCAGAGCGAGACGCTGCCGAGACGGACGGCGTCGATGAGGATCAG
GTCGGTGGCATCGACGGCGGTGCCCTGCTGGGAGGAGAGGGAGATGGCGGTGAGGAGGC
GGGGAGATTTGGGCTGGTACCGCCACCTTGACATGGCAGTGCTCGACGGCACCGTCGTA
AGGCTGTGGGACAGCGAGGTCACCGCGGCGGTGGCGTCGCCGAGGGCGCGGCGGGGGAG
GGCAGGATTGGAACTGCACCTGTCACTATAGAGATGGAGATCCTGATGTGTATAATGTG
GTTAGGATTATTGGACTGTGATTAGGTGGGTATGACAGTTTTGTCCTTCAATTTGGGCA
TGTAATATGTATGTGTATCATCGAAGAGGGCCGAGGCTGAAAAAATGAACAGCCTTCGT
CTCAGCTCTCAGGAGAAATAAAAATTTGGCAAATGGCAGAGTATTCTTACT

A RFS amino acid sequence encoded by the nucleotide sequence of SEQ ID NO: 32 (TRIAE_CS42_5BL_TGACv1_407440_AA1356920) is shown below as SEQ ID NO: 33.

MDLLNVVPAD AMAFPLYSLP AAANTVASLF AWLVAALAAA
VGLWRIRAVG SSNKLPDAGA RSSTLVDDKQ QMQALSSPAA
DEPRPARTVP VEPASPISEP SSPSKVRFTA YYGGAGADAG
NDGVVDGVRK CADRDEDEDG VPVVDDQSET LPRRTASMRI
RSVASTAVPC WEEREMAVRR RGDLGWYRHL DMAVLDGTVV
RLWDSEVTAA VASPRARRGR AGLELHLSL

A RFS nucleotide sequence from Triticum aestivum (TRIAE_CS42_5DL_TGACv1_434454_AA1436290) is shown below as SEQ ID NO: 34.

ACCCTCCTCACTATCCTCATCATTCACAACAAACCCAAAAACCAAACCTCCTCCTTCCT
ATCTTGAACAACCGCCGAGAGACCTCCCCGTCGCCGTCGCCGCAAATGGAGCTGCTCAA
CGTGGTTCCGGCGGACGCCATGGCCTTCCCCCTCTACTCCCTCCCCGCGGCGGCCAACA
CCGTCGCCTCGCTCTTCGCCTGGCTCGTCGCGGCCCTCGCCGCCGCCGTCGGCCTCTGG
CGCATCCGCGCGGCCGGCTCCTCCAACAAACTACCCGGCGCCGGCGCCCGCAGCAGCAC
CCTCTTGGACGACAATCAGCAGCCGCAGGCCGTTTCGTCGTCTGCCGCCGACGAGCCAC
GGCCCGCACTCGCCGTGCCGGTGGAGCCGTCCTCCCCGATTAGCGAGCCGAGCTCGCCG
TCCAAGGTCCGGTTCACGGCATACTACGGCGGGGCCGGAGCTGACGATGGGGTAGTGGA
CGGCGTCAGGAAATGCGCGGACAGGGACGAGGACGAGGACGGCGTGCCCGTCGTCGACG
ACCAGAGCGAGACACAGCCGAGACGGACGGCGTCGATGAGGATCAGGTCGGCAGCCTCG
ACGGCGGTGCCCTGCTGGGAGAAGAGGGAGATGGCCGTGAGGAGGCGGGGAGATTTGGG
CTGGTACCGCCACCTTGACACGGCGGTGCTTGACGGCACCGTTGTAAGGCTGTGGGACG
GCGAGGTCACCGCGGCGGTGGCGTCGCCGAGGGCGCGGCGGCGGAGGGCAGGATTGAAA
CCGCGCCTGTCACTATAGAGATGTGTGTATAATGTGGTTAGGATTATTGGATTGTTATT
AGGTGGGTATGACGATTTTGTCCTTCAATTTGGGCATGTCATATGTATGTGTATCGTCT
AAGAGGGCCGAGGCTGAAAAAATGAACAGCCTTCGTCTCAGCTCTCAGGAGAAACGAAA
ATTTGGCAAATAATGGCAGAGTATTCTTGCT

A RFS amino acid sequence encoded by the nucleotide sequence of SEQ ID NO: 34 (TRIAE_CS42_5DL_TGACv1_434454_AA1436290) is shown below as SEQ ID NO: 35.

MELLNVVPAD AMAFPLYSLP AAANTVASLF AWLVAALAAA
VGLWRIRAAG SSNKLPGAGA RSSTLLDDNQ QPQAVSSSAA
DEPRPALAVP VEPSSPISEP SSPSKVRFTA YYGGAGADDG
VVDGVRKCAD RDEDEDGVPV VDDQSETQPR RTASMRIRSA
ASTAVPCWEK REMAVRRRGD LGWYRHLDTA VLDGTVVRLW
DGEVTAAVAS PRARRRRAGL KPRLSL

Additional RFS-like homologs identified by BLAST search using SEQ ID NO: 1 found in plant species that are water-intensive crops, such as rice, soybeans, wheat, sugarcane, cotton, alfalfa, and pasture.

The nucleic acids and polypeptides allow identification and isolation of related nucleic acids and their encoded enzymes that provide a means for production of healthy plants with drought tolerance, delayed flowering time, and increased biomass.

The related nucleic acids can be isolated and identified by mutation of the SEQ ID NOS: 2, 3, 6, 8, 10, 12, 14, 16, 18, 20, 22, 24, 26, 28, 30, 32, or 34 nucleic acid sequences and/or by hybridization to DNA and/or RNA isolated from other plant species using segments of these nucleic acids as probes. The sequence of the RFS proteins (e.g., SEQ ID NOS: 1, 4, 5, 7, 9, 11, 13, 15, 17, 19, 21, 23, 25, 27, 29, 31, 33, and 35) can also be examined and used a basis for designing alternative RFS nucleic acids that encode related RFS polypeptides.

The RFS nucleic acids described herein can include any nucleic acid that can selectively hybridize to any of SEQ ID NOS: 2, 3, 6, 8, 10, 12, 14, 16, 18, 20, 22, 24, 26, 28, 30, 32, or 34 nucleic acids.

The term “selectively hybridize” includes hybridization, under stringent hybridization conditions, of a nucleic acid sequence to a specified nucleic acid target sequence (e.g., any of the SEQ ID NOS: 2, 3, 6, 8, 10, 12, 14, 16, 18, 20, 22, 24, 26, 28, 30, 32, or 34 nucleic acids) to a detectably greater degree (e.g., at least 2-fold over background) than its hybridization to non-target nucleic acid sequences. Such selective hybridization substantially excludes non target nucleic acids. Selectively hybridizing sequences typically have about at least 40% sequence identity, or at least 50% sequence identity, or at least 60% sequence identity, or at least 70% sequence identity, or at least 80% sequence identity, or at least 90% sequence identity, or at least 95% sequence identity, or at least 96% sequence identity, or at least 97% sequence identity, or at least 98% sequence identity, or at least 99% sequence identity, or 60-99% sequence identity, or 70-99% sequence identity, or 80-99% sequence identity, or 90-95% sequence identity, or 90-99% sequence identity, or 95-97% sequence identity, or 97-99% sequence identity, or 100% sequence identity (or complementarity) with each other. In some embodiments, a selectively hybridizing sequence has about at least about 80% sequence identity or complementarity with SEQ ID NOS: 2, 3, 6, 8, 10, 12, 14, 16, 18, 20, 22, 24, 26, 28, 30, 32, and 34.

Thus, the nucleic acids of the invention include those with about 500 of the same nucleotides as SEQ ID NOS: 2, 3, 6, 8, 10, 12, 14, 16, 18, 20, 22, 24, 26, 28, 30, 32, or 34 or about 600 of the same nucleotides, or about 700 of the same nucleotides, or about 800 of the same nucleotides, or about 900 of the same nucleotides, or about 1000 of the same nucleotides, or about 1100 of the same nucleotides, or about 1200 of the same nucleotides as SEQ ID NO: 2, 3, 6, 8, 10, 12, 14, 16, 18, 20, 22, 24, 26, 28, 30, 32, and 34. The identical nucleotides or amino acids can be distributed throughout the nucleic acid or the protein, and need not be contiguous.

Note that if a value of a variable that is necessarily an integer, e.g., the number of nucleotides or amino acids in a nucleic acid or protein, is described as a range. e.g., 90-99% sequence identity what is meant is that the value can be any integer between 90 and 99 inclusive, i.e., 90, 91, 92, 93, 94, 95, 96, 97, 98 or 99, or any range between 90 and 99 inclusive, e.g., 91-99%, 91-98%, 92-99%, etc.

The terms “stringent conditions” or “stringent hybridization conditions” include conditions under which a probe will hybridize to its target sequence to a detectably greater degree than other sequences (e.g., at least 2-fold over background).

Stringent conditions are somewhat sequence-dependent and can vary in different circumstances. By controlling the stringency of the hybridization and/or washing conditions, target sequences can be identified with up to 100% complementarity to the probe (homologous probing). Alternatively, stringency conditions can be adjusted to allow some mismatching in sequences so that lower degrees of sequence similarity are detected (heterologous probing). The probe can be approximately 20-500 nucleotides in length but can vary greatly in length from about 18 nucleotides to equal to the entire length of the target sequence. In some embodiments, the probe is about 10-50 nucleotides in length, or about 18-25 nucleotides in length, or about 18-50 nucleotides in length, or about 18-100 nucleotides in length.

Typically, stringent conditions will be those where the salt concentration is less than about 1.5 M Na⁻ ion (or other salts), typically about 0.01 to 1.0 M Na⁺ ion concentration (or other salts), at pH 7.0 to 8.3 and the temperature is at least about 30° C. for shorter probes (e.g., 10 to 50 nucleotides) and at least about 60° C. for longer probes (e.g., greater than 50 nucleotides). Stringent conditions may also be achieved with the addition of destabilizing agents such as formamide or Denhardt's solution. Exemplary low stringency conditions include hybridization with a buffer solution of 30 to 35% formamide, 1M NaCl, 1% SDS (sodium dodecyl sulfate) at 37° C., and a wash in 1×SSC to 2×SSC (where 20×SSC is 3.0 M NaCl, 0.3 M trisodium citrate) at 50 to 55° C. Exemplary moderate stringency conditions include hybridization in 40 to 45% formamide, 1M NaCl, 1% SDS at 37° C., and a wash in 0.5×SSC to 1×SSC at 55 to 60° C. Exemplary high stringency conditions include hybridization in 50% formamide, 1M NaCl, 1% SDS at 37° C., and a wash in 0.1×SSC at 60 to 65° C. Specificity is typically a function of post-hybridization washes, where the factors controlling hybridization include the ionic strength and temperature of the final wash solution. Thus, high stringency conditions can include a wash that includes 0.1×SSC at 60 to 65° C.

For DNA-DNA hybrids, the Tₘ can be approximated from the equation of Meinkoth and Wahl (Anal. Biochem. 138:267-84 (1984)):

Tₘ=81.5° C.+16.6 (log M)+0.41 (% GC)−0.61 (% formamide)−500/L where M is the molarity of monovalent cations; % GC is the percentage of guanosine and cytosine nucleotides in the DNA, % formamide is the percentage of formamide in the hybridization solution, and L is the length of the hybrid in base pairs. The Tₘ is the temperature (under defined ionic strength and pH) at which 50% of a complementary target sequence hybridizes to a perfectly matched probe. The Tₘ is reduced by about 1° C. for each 1% of mismatching. Thus, the Tₘ, hybridization and/or wash conditions can be adjusted to hybridize to sequences of the desired sequence identity. For example, if sequences with greater than or equal to 90% sequence identity are sought, the Tₘ can be decreased 10° C. Generally, stringent conditions are selected to be about 5° C. lower than the thermal melting point (Tₘ) for the specific sequence and its complement at a defined ionic strength and pH.

However, severely stringent conditions can include hybridization and/or a wash at 1, 2, 3 or 4° C. lower than the thermal melting point (Tₘ). Moderately stringent conditions can include hybridization and/or a wash at 6, 7, 8, 9 or 10° C. lower than the thermal melting point (Tₘ). Low stringency conditions can include hybridization and/or a wash at 11, 12, 13, 14, 15 or 20° C. lower than the thermal melting point (Tₘ). Using the equation, hybridization and wash compositions, and a desired Tₘ, those of ordinary skill can identify and isolate nucleic acids with sequences related to any of SEQ ID NOS: 2, 3, 6, 8, 10, 12, 14, 16, 18, 20, 22, 24, 26, 28, 30, 32, or 34.

Those of skill in the art also understand how to vary the hybridization and/or wash solutions to isolate desirable nucleic acids. For example, if the desired degree of mismatching results in a Tₘ of less than 45° C. (aqueous solution) or 32° C. (formamide solution), it may be preferred to increase the SSC concentration so that a higher temperature can be used.

An extensive guide to the hybridization of nucleic acids is found in Tijssen, LABORATORY TECHNIQUES IN BIOCHEMISTRY AND MOLECULAR BIOLOGY—HYBRIDIZATION WITH NUCLEIC ACID PROBES, part 1, chapter 2, “Overview of principles of hybridization and the strategy of nucleic acid probe assays.” Elsevier. N.Y. (1993); and in CURRENT PROTOCOLS IN MOLECULAR BIOLOGY, chapter 2, Ausubel, et ah, eds. Greene Publishing and Wiley-Interscience, New York (1995).

Unless otherwise stated, in the present application high stringency is defined as hybridization in 4×SSC, 5×Denhardt's (5 g Ficoll, 5 g polyvinylpyrrolidone, 5 g bovine serum albumin in 500 ml of water), 0.1 mg/ml boiled salmon sperm DNA, and 25 mM Na phosphate at 65° C. and a wash in 0.1×SSC, 0.1% SDS at 65° C.

However, because specificity is typically a function of post-hybridization washes, where the factors controlling hybridization include the ionic strength and temperature of the final wash solution, the high stringency conditions can more simply be expressed as including a wash in 0.1×SSC at 60 to 65° C.

The following terms are used to describe the sequence relationships between two or more nucleic acids or polypeptides: (a) “reference sequence.” (b) “comparison window,” (c) “sequence identity,” (d) “percentage of sequence identity” and (e) “substantial identity.”

As used herein, “reference sequence” is a defined sequence used as a basis for sequence comparison. The reference sequence can be a nucleic acid sequence (e.g., any of SEQ ID NOS: 2, 3, 6, 8, 10, 12, 14, 16, 18, 20, 22, 24, 26, 28, 30, 32, or 34) or an amino acid sequence (e.g., any of SEQ ID NOS: 1, 4, 5, 7, 9, 11, 13, 15, 17, 19, 21, 23, 25, 27, 29, 31, 33, or 35). A reference sequence may be a subset or the entirety of a specified sequence. For example, a reference sequence may be a segment of a full-length cDNA or of a genomic DNA sequence, or the complete cDNA or complete genomic DNA sequence, or a domain of a polypeptide sequence.

As used herein, “comparison window” refers to a contiguous and specified segment of a nucleic acid or an amino acid sequence, wherein the nucleic acid/amino acid sequence can be compared to a reference sequence and wherein the portion of the nucleic acid/amino acid sequence in the comparison window may comprise additions or deletions (i.e., gaps) compared to the reference sequence (which does not comprise additions or deletions) for optimal alignment of the two sequences. The comparison window can vary for nucleic acid and polypeptide sequences. Generally, for nucleic acids, the comparison window is at least 20 contiguous nucleotides in length, and optionally can be 30, 40, 50, 100 or more nucleotides. For amino acid sequences, the comparison window is at least about 10 amino acids, and can optionally be 15, 20, 30, 40, 50, 100 or more amino acids. Those of skill in the art understand that to avoid a high similarity to a reference sequence due to inclusion of gaps in the nucleic acid or amino acid sequence, a gap penalty is typically introduced and is subtracted from the number of matches.

Methods of alignment of nucleotide and amino acid sequences for comparison are well known in the art. The local homology algorithm (BESTFIT) of Smith and Waterman, (1981) Adv. Appl. Math 2:482, may permit optimal alignment of compared sequences; by the homology alignment algorithm (GAP) of Needleman and Wunsch, (1970) J. Mol. Biol. 48:443-53; by the search for similarity method (Tfasta and Fasta) of Pearson and Lipman, (1988) Proc. Natl. Acad. Sci. USA 85:2444; by computerized implementations of these algorithms, including, but not limited to: CLUSTAL in the PC/Gene program by Intelligenetics, Mountain View, Calif., GAP, BESTFIT, BLAST, FASTA and TFASTA in the Wisconsin Genetics Software.

Package, Version 8 (available from Genetics Computer Group (GCG™ programs (Accelrys, Inc., San Diego, Calif.)). The CLUSTAL program is well described by Higgins and Sharp (1988) Gene 73:237-44; Higgins and Sharp, (1989) CABIOS 5: 151-3; Corpet, et al., (1988) Nucleic Acids Res. 16: 10881-90; Huang, et al., (1992) Computer Applications in the Biosciences 8:155-65 and Pearson, et al., (1994) Meth. Mol. Biol. 24:307-31. An example of a good program to use for optimal global alignment of multiple sequences is PileUp (Feng and Doolittle, (1987) J. Mol. Evol., 25:351-60, which is similar to the method described by Higgins and Sharp, (1989) CABIOS 5:151-53 (and is hereby incorporated by reference). The BLAST family of programs that can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., eds., Greene Publishing and Wiley-Interscience, New York (1995).

GAP uses the algorithm of Needleman and Wunsch, (1970) J. Mol. Biol. 48:443-53, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP makes a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package are 8 and 2, respectively. The gap creation and gap extension penalties can be expressed as an integer selected from the group of integers consisting of from 0 to 100. Thus, for example, the gap creation and gap extension penalties can be 0, 1, 2, 3, 4, 5, 6, 7, 8, 9, 10, 15, 20, 30, 40, 50 or more.

GAP presents one member of the family of best alignments. There may be many members of this family. GAP displays four figures of merit for alignments: Quality, Ratio. Identity and Similarity. The Quality is the metric maximized to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see, Henikoff and Henikoff, (1989) Proc. Natl. Acad. Sci. USA 89:10915).

For example, sequence identity/similarity values provided herein can refer to the value obtained using the BLAST 2.0 suite of programs using default parameters (Altschul, et ah, (1997) Nucleic Acids Res. 25:3389-402).

BLAST searches assume that proteins can be modeled as random sequences. However, many real proteins comprise regions of nonrandom sequences, which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, (1993) Comput. Chem. 17:149-63) and XNU (Ci-ayerie and States, (1993) Comput. Chem. 17:191-201) low-complexity filters can be employed alone or in combination.

The terms “substantial identity” indicates that a polypeptide or nucleic acid comprises a sequence with between 55-100% sequence identity to a reference sequence, with at least 55% sequence identity, or at least 60%, or at least 70%, or at least 80%, or at least 90% or at least 95% sequence identity, or at least 96%, or at least 97%, or at least 98%, or at least 99%, or any percentage value within the range of 55-100% sequence identity relative to the reference sequence over a specified comparison window. Optimal alignment may be ascertained or conducted using the homology alignment algorithm of Needleman and Wunsch, supra.

Alternatively, substantial identity is present when a second polypeptide is immunologically reactive with antibodies raised against the first polypeptide (e.g., a polypeptide with SEQ ID NOS: 1, 4, 5, 7, 9, 11, 13, 15, 17, 19, 21, 23, 25, 27, 29, 31, 33, or 35). Thus, a polypeptide is substantially identical to a first polypeptide, for example, where the two polypeptides differ only by a conservative substitution. In addition, a polypeptide can be substantially identical to a first polypeptide when they differ by a non-conservative change if the epitope that the antibody recognizes is substantially identical. Polypeptides that are “substantially similar” share sequences as noted above except that some residue positions, which are not identical, may differ by conservative amino acid changes.

The RFS polypeptide can include the first 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 and 99 N-terminal amino acid residues of a the SEQ ID NOS: 1, 4, 5, 7, 9, 11, 13, 15, 17, 19, 21, 23, 25, 27, 29, 31, 33, and 35 sequence. Alternatively, the RFS polypeptides may include the first 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 and 99 C-terminal amino acid residues of the SEQ ID NOS: 1, 4, 5, 7, 9, 11, 13, 15, 17, 19, 21, 23, 25, 27, 29, 31, 33, or 35 sequence.

### Plants Modified to Express or Contain RFS

To engineer healthy plants with increased tolerance to drought conditions and/or delayed flowering and increased biomass, one of skill in the art can introduce RFS, or nucleic acids encoding such RFS polypeptides into the plants. Introduction of RFS, or expression of increased levels of RFS, in a plant can increase the plant's biomass by approximately 20% or more. For example, introduction of RFS, or expression of increased levels of RFS, in a plant can increase the plant's biomass by at least 10%, or at least 15%, or at least 20%, or at least 25%, or at least 30%, or at least 33% compared to a wild-type plant of the same species that does not comprise the RFS expression cassette.

For example, one of skill in the art can inject RFS polypeptides into young plants.

Alternatively, one of skill in the art can generate genetically-modified plants that contain nucleic acids encoding RFS within their somatic and/or germ cells. Such genetic modification can be accomplished by various procedures.

Non-limiting examples of methods of introducing a modification into the genome of a plant cell can include microinjection, viral delivery, recombinase technologies, homologous recombination, TALENS, CRISPR, and/or ZFN, see, e.g. Clark and Whitelaw Nature Reviews Genetics 4:825-833 (2003); which is incorporated by reference herein in its entirety.

For example, nucleases such as zinc finger nucleases (ZFNs), transcription activator like effector nucleases (TALENs), and/or meganucleases can be employed with guide nucleic acid that allows the nuclease to target the genomic RFA site(s). In some cases of the various aspects described herein, a targeting vector can be used to introduce a transcriptional element, promotor, deletion, or modification of the genomic RFS chromosomal sites.

A “targeting vector” is a vector generally has a 5′ flanking region and a 3′ flanking region homologous to segments of the gene of interest. The 5′ flanking region and a 3′ flanking region can surround a DNA sequence comprising a modification and/or a foreign DNA sequence to be inserted into the gene. For example, foreign DNA to be inserted may encode a non-native promotor (as described below) and/or a selectable marker, such as an antibiotics resistance gene. Examples for suitable selectable markers include chloramphenicol resistance, gentamycin resistance, kanamycin resistance, spectinomycin resistance (SpecR), neomycin resistance gene (NEO) and hygromycin β-phosphotransferase markers (genes). The 5′ flanking region and the 3′ flanking region can be homologous to regions within the gene, or such flanking regions can flank the coding region of gene to be deleted, mutated, or replaced with the unrelated DNA sequence. In some cases, the targeting vector does not comprise a selectable marker. DNA comprising the targeting vector and the native gene of interest are contacted under conditions that favor homologous recombination (e.g., by transforming plant cell(s) with the targeting vector).

A typical targeting vector contains nucleic acid fragments of not less than about 0.1 kb nor more than about 10.0 kb from both the 5′ and the 3′ ends of the genomic locus which encodes the gene to be modified (e.g. the genomic RFS site(s)). These two fragments can be separated by an intervening fragment of nucleic acid that includes the modification to be introduced. When the resulting construct recombines homologously with the chromosome at this locus, it results in the introduction of the modification, e.g. an insertion, substitution, or a deletion of a portion of the genomic RFS site(s).

In some cases, a Cas9/CRISPR system can be used to create a modification in genomic RFS site(s). Clustered regularly interspaced short palindromic repeats (CRISPR)/CRISPR-associated (Cas) systems are useful for, e.g. RNA-programmable genome editing (see e.g., Marraffini & Sontheimer. Nature Reviews Genetics 11: 181-190 (2010); Sorek et al. Nature Reviews Microbiology 2008 6: 181-6; Karginov and Hannon. Mol Cell 2010 1:7-19; Hale et al. Mol Cell 2010:45:292-302; Jinek et al. Science 2012 337:815-820; Bikard and Marraffini Curr Opin Immunol 2012 24:15-20; Bikard et al. Cell Host & Microbe 2012 12: 177-186; all of which are incorporated by reference herein in their entireties). A CRISPR guide RNA can be used that can target a Cas enzyme to the desired location in the genome, where it generates a double strand break. This technique is available in the art and described, e.g. at Mali et al. Science 2013 339:823-6; which is incorporated by reference herein in its entirety and kits for the design and use of CRISPR-mediated genome editing are commercially available, e.g. the PRECISION X CAS9 SMART NUCLEASE™ System (Cat No. CAS900A-1) from System Biosciences. Mountain View, CA.

In another example, one of skill in the art can prepare an expression cassette or expression vector that can express one or more encoded RFS polypeptides. Plant cells can be transformed by the expression cassette or expression vector, and whole plants (and their seeds) can be generated from the plant cells that were successfully transformed with the RFS nucleic acids. Some procedures for making such genetically modified plants and their seeds are described below.

Promoters: The RFS nucleic acids described herein can be operably linked to a promoter, which provides for expression of mRNA from the RFS nucleic acids. The promoter can be a promoter functional in plants and/or seeds and can be a promoter functional during plant growth and development. In some cases, the promotor can be a non-native promotor that is not found in the plant cell, plant seed, or plant the RFS nucleic acid is expressed in. A RFS nucleic acid is operably linked to the promoter when it is located downstream from the promoter, to thereby form an expression cassette.

Most endogenous genes have regions of DNA that are known as promoters, which regulate gene expression. Promoter regions are typically found in the flanking DNA upstream from the coding sequence in both prokaryotic and eukaryotic cells. A promoter sequence provides for regulation of transcription of the downstream gene sequence and typically includes from about 50 to about 2,000 nucleotide base pairs. Promoter sequences also contain regulatory sequences such as enhancer sequences that can influence the level of gene expression. Some isolated promoter sequences can provide for gene expression of heterologous DNAs, that is a DNA different from the native or homologous DNA.

Promoter sequences are also known to be strong or weak, or inducible. A strong promoter provides for a high level of gene expression, whereas a weak promoter provides for a very low level of gene expression. An inducible promoter is a promoter that allows gene expression to be turned on and off in response to an exogenously added agent, or to an environmental or developmental stimulus. For example, a bacterial promoter such as the P_{tac} promoter can be induced to vary levels of gene expression depending on the level of isothiopropylgalactoside added to the transformed cells. Promoters can also provide for tissue specific or developmental regulation. An isolated promoter sequence that is a strong promoter for heterologous DNAs is advantageous because it provides for a sufficient level of gene expression for easy detection and selection of transformed cells and provides for a high level of gene expression when desired.

Expression cassettes generally include, but are not limited to, a plant promoter such as the CaMV 35S promoter (Odell et ah, Nature. 313:810-812 (1985)), or others such as CaMV 19S (Lawton et ah, Plant Molecular Biology. 9:315-324 (1987)), nos (Ebert et ah, Proc. Natl. Acad. Sci. USA. 84:5745-5749 (1987)), Adhl (Walker et ah, Proc. Natl. Acad. Sci. USA. 84:6624-6628 (1987)), sucrose synthase (Yang et ah, Proc. Natl. Acad. Sci. USA. 87:4144-4148 (1990)), a-tubulin, ubiquitin, actin (Wang et al., Mol. Cell. Biol. 12:3399 (1992)), cab (Sullivan et ah, Mol. Gen. Genet. 215:431 (1989)), PEPCase (Hudspeth et al., Plant Molecular Biology. 12:579-589 (1989)) or those associated with the R gene complex (Chandler et al., The Plant Cell. 1: 1175-1183 (1989)). Further suitable promoters include the poplar xylem-specific secondary cell wall specific cellulose synthase 8 promoter, cauliflower mosaic virus promoter, the Z10 promoter from a gene encoding a 10 kDa zein protein, a Z27 promoter from a gene encoding a 27 kDa zein protein, inducible promoters, such as the light inducible promoter derived from the pea rbcS gene (Coruzzi et al., EMBO J. 3: 1671 (1971)) and the actin promoter from rice (McElroy et al., The Plant Cell. 2: 163-171 (1990)). Seed specific promoters, such as the phaseolin promoter from beans, may also be used (Sengupta-Gopalan, Proc. Natl. Acad. Sci. USA. 83:3320-3324 (1985).

The promotor can include a drought-regulated promoter. For example, the drought-regulated promotor can include a 7DA2 promoter as disclosed in U.S. Pat. No. 10,106,813, issued Oct. 23, 2018 to Han, K. H., et. al., and incorporated herein by reference.

The promotor can include a cold-stress-responsive promoter for delayed flowering, such as to avoid flowering during cold temperatures. For example, the cold-stress-responsive promotor can be a Cor15A promotor. (Baker, S. et. al., The 5′-region of Arabidopsis thaliana cor15a has cis-acting elements that confer cold-, drought- and ABA-regulated gene expression, Plant Mol. Biol. 24: 701-713 (1994), incorporated herein by reference).

Another promoter useful for expression of RFS is the Brachypodium distachyon PIN-like (e.g., PIN-4) promoter, which can have the sequence shown below (SEQ ID NO: 36).

| | |
|---|---|
| 1 | GATTTGAGCA TGTTCTTGAT GAGGTCCTTG GCGCTGGGGG |
| 41 | AGATGTTGGG CCACGGGTCG GAGTCGAAGT CTATGGCGCC |
| 81 | TTTTAGGACC GCGTCGAAGA TCCCCTGCTG CGTCTCGGCC |
| 121 | CAGAAGGGCG GGACGCCGGA GAGCAGGATG TAGACGATGA |
| 161 | CCCCCGCCGT CCAGACGTCG GCTTCGGGCC CGTAGTGCTT |
| 201 | GCAGAGGACC TCGGGGGCCA CGTAGTACGG GCTTCCGACG |
| 241 | ACGTCGGTGA AGATCTGGCC GGGCTTGAAG AAGACGGAGA |
| 281 | GTCCGAAATC GATGGCCTTG AGATCGGCGA CCGAGTCGTC |
| 321 | TTCGTCTTCT CCGTTGCCGG CGCCGGCGCC GCCGAGCAAG |
| 361 | AGGAAGTTCT CGGGCTTGAG GTCGCGGTGC ATGACCCCCA |
| 401 | GAGAATGGCA CGCCTCGACG ACGCCGACGA CGACGCGTGC |
| 441 | GATCTCGGCG GCTTTCCGCT CGGAGAAGTA TCCGCGGGCG |
| 481 | ACGATGCGGT CGAAGAGCTC GCCGCCCTCG CAGAGGTCCA |
| 521 | TGACGATGTG GACGTAGAGC GGGTCCTCGT AGGCGCCGCG |
| 561 | GATGGTGACG ACGCTGGCGT GGCCCGCCAG GTGGTGCATG |
| 601 | ATCTGGATCT CGCGGCGGAC GTCGTCCACG TCCTCGGGGG |
| 641 | TGAGGAGCTT GCGCTTGGGG ATGGACTTGC AGGCGAGGGG |
| 681 | TGTCCCCGTG GCGATGTCGG TGCAGAGGTA GGTGGTGCCG |
| 721 | AACTGGCCCT GGCCGAGCTT GCGGCCGAGC GTGTAGAGGG |
| 761 | AGGTGAGCGG CGGGGTGTCG TGGCCGAGGA CGGCGGTCGG |
| 801 | GGAGGAGAGG TGGTGCTGGT GGCCGCGCAT GGTGTTGGTG |
| 841 | GTGCAGGGGG CTTGGAGGTG GAGATGGAAG GGGTCCGAGT |
| 881 | CGGCGGTGCT GCTGTTGGAA TCGCGGCACG AGTAGTTGCC |
| 921 | CATGCGCACC GCGTCAATTG TCGCCGGCGG CCATGGCGAC |
| 961 | CACCGTGGAT GGATGATTGG ACCACAGAGA AATTAGGGGG |
| 1001 | TGGAGAGGAA GAGGAGAGCT GTGCTCCATT AGTTTGGGAG |
| 1041 | GAAGAGGAGA CCAAATTGGC AATGGCCTGC ATGTCGTGCG |
| 1081 | CTGCACCTAC CTAAGCTAGC GTGCATGTCG ATTTGCTCCT |
| 1121 | GCGACACCAC GATTCGGCCC TTTTTCGGCC TAAATGAAAC |
| 1161 | ATCGTCCATC TCGAATCAAC CTAGCCACAT CATTCTTTTT |
| 1201 | CTTTTTGCAA GATCGATCCC TGTGCAGTAG ACATGCATGC |
| 1241 | TGGAGTAGCA GTAGGAATCA GGGACTGGCC AGCCTGGCCT |
| 1281 | TGCTAGTGAG CGAGTGTACG TGCAATGCCA ATTAACCGTT |
| 1321 | TGCTTATTTT ACTAGTACCA TCATATCGAT CGATCTCAAT |
| 1361 | CAAGCTGCTG ACGTAGGGCA ACATATATAA GATCGTTTTC |
| 1401 | AGCTCGTGGT GCACGATGCG CAATAATACC GATCCTGTTA |
| 1441 | GTTGAGTTCA ATCAATTAAG AGCTCTGTTT CCTCATCTCT |
| 1481 | CACCTACGAG AAGCGGCGCA TACAGAAATA GAAGATGTTG |
| 1521 | AGGTAGATCA AGTTCATATT GATGTTAACT TGAATACTTA |
| 1561 | TTGAAGATTT CAATTCAAAG GACACTAGAA GAATGATGCT |
| 1601 | GTTCAAATAA AGATGTTGAG GTAGAGGAAG TTCATTATTC |
| 1641 | TAGTACTTTT CTAGTGAGGG AGATTTTCGC ACCTGCATGT |
| 1681 | ATTTATTGCT GTCAAATATA TGACGCCAAT GAAATAGAAA |
| 1721 | AATACTCTTA ATTAATAATA TGCGATAATA AATTATTTTA |
| 1761 | CCCCGGCCGG TGGTTTATTT TTCTTGCTTC GCGCCCCTGC |
| 1801 | CTAGCGAGGA GAGGTGCATG CGATCCACCG GCCCATGGAT |
| 1841 | CGTCGCTTAA TTAGTACCGG TAATTTCCTT ATTAAACCAG |
| 1881 | GAATGCAAAT AATTCATGTC CTGGACAGTG AGATGATGAG |
| 1921 | CAGGTCGGCG GGTATGCGCG CGAACGTACG GTCTCTGTCG |
| 1941 | ATCGTGTGCC ACGTGCATTA GCGGAGCCGA CGGCCTGCTC |
| 1961 | GCAGAGCCCG GACAAATTCC CTAAAAATTA ATTATACAAG |
| 2001 | AAAAACACTA CTCTGGTGGC TAATTAACAC GCTGGCTAGC |
| 2041 | GGCATCATGG CTTCCCCAGT GATCGATAGC ACTGGGGAAG |
| 2081 | CATGCATAGC TCGATGGAAT CACTCCATGC GAGTGCATAT |
| 2121 | GTCGCACCAA CCAAATTTCT TTCGTCACTT AGTATGAAAC |
| 2161 | GGAGAGAATG TATGATCGAC CGATTCTGAT CCCGCATGAT |
| 2201 | AATAGTGAGA TCGATTCTGG TCCCGCATGA TAATAATGAG |
| 2241 | ATCTCAACAA ATTAACCAAC AAACATACAA TTGCACATGC |
| 2281 | CTGCCTATAC TACTTATCAC CGTCCAAATT AAAGCATTCA |
| 2321 | TGCCACCCTA GCTAAAAATA GATACATCCA TATTTAAACA |
| 2361 | AATTTGAATT AAGAATTTAG AAACGGGAGC AGGCAGGAAC |
| 2401 | AATCCAGCGG CTTCTTATTG ACTCTGTCAA CACAACACTA |
| 2441 | GCTAGCTGGG TTTTCAGACT TCATTAACAG CGCACGCTAG |
| 2481 | CGGCATCATG GCTTCCCAAG TGAGCGGTCG AGCGCCGACA |
| 2521 | AAAACGGGAC CCCGGCCCTC TGTGTGATTT GATGCGAGTT |
| 2561 | GCTAGCAGTGT GTCTGACAC TGTGATGTTT GGTCCAGGTA |
| 2601 | TGAACCAACC AAGATCACAG GAAAAAAAAC AATCGCACAT |
| 2641 | GCATGTATGA ATCTCCTCCG GCCTATATAT ACTCGCCACC |
| 2681 | ATCTCGGAAT TAAAGCATGC ATGCCACTTA CAGCAGGCTT |
| 2721 | GCATCACCAG CTGCCACTCA GCTGGGTTTT CATCAGTCTT |
| 2761 | AAACTGAGCT GTGTTAATTA CCTGAGCACA CACACAGCTC |
| 2801 | AAGTCTGAAC AAGCTAGTAA G |

Alternatively, novel tissue specific promoter sequences may be employed in the practice of the present invention. cDNA clones from a particular tissue can be isolated and those clones which are expressed specifically in that tissue are identified, for example, using Northern blotting. Preferably, the gene isolated is not present in a high copy number but is relatively abundant in specific tissues. The promoter and control elements of corresponding genomic clones can then be localized using techniques well known to those of skill in the art.

A RFS nucleic acid can be combined with the promoter by standard methods to yield an expression cassette, for example, as described in Sambrook et al. (MOLECULAR CLONING: A LABORATORY MANUAL. Second Edition (Cold Spring Harbor. NY: Cold Spring Harbor Press (1989); MOLECULAR CLONING: A LABORATORY MANUAL. Third Edition (Cold Spring Harbor, NY: Cold Spring Harbor Press (2000)). Briefly, a plasmid containing a promoter such as the 35 S CaMV promoter can be constructed as described in Jefferson (Plant Molecular Biology Reporter 5:387-405 (1987)) or obtained from Clontech Lab in Palo Alto, California (e.g., pBI121 or pBI221). Typically, these plasmids are constructed to have multiple cloning sites having specificity for different restriction enzymes downstream from the promoter. The RFS nucleic acids can be subcloned downstream from the promoter using restriction enzymes and positioned to ensure that the DNA is inserted in proper orientation with respect to the promoter so that the DNA can be expressed as sense RNA. Once the RFS nucleic acid sequence is operably linked to a promoter, the expression cassette so formed can be subcloned into a plasmid or other vector (e.g., an expression vector).

In some embodiments, a cDNA clone encoding a RFS protein is isolated from plant tissue, for example, a root, stem, leaf, seed, or flower tissue. For example, cDNA clones from selected species (that encode a RFS protein with homology to any of those described herein) are made from isolated mRNA from selected plant tissues. In another example, a nucleic acid encoding a mutant or modified RFS protein can be prepared by available methods or as described herein. For example, the nucleic acid encoding a mutant or modified RFS protein can be any nucleic acid with a coding region that hybridizes to a segment of a SEQ ID NOS: 2, 3, 6, 8, 10, 12, 14, 16, 18, 20, 22, 24, 26, 28, 30, 32, or 34 nucleic acid. Such a nucleic acid can encode an enzyme with glucan synthase activity and/or protein folding activity. Using restriction endonucleases, the entire coding sequence for the modified RFS is subcloned downstream of the promoter in a 5′ to 3′ sense orientation.

Targeting Sequences: Additionally, expression cassettes can be constructed and employed to target the RFS proteins to an intracellular compartment within plant cells, into a membrane, or to direct an encoded protein to the extracellular environment. This can generally be achieved by joining a DNA sequence encoding a transit or signal peptide sequence to the coding sequence of the RFS nucleic acid. The resultant transit, or signal, peptide will transport the protein to a particular intracellular, or extracellular destination, respectively, and can then be posttranslational removed. Transit peptides act by facilitating the transport of proteins through intracellular membranes, e.g., vacuole, vesicle, plastid and mitochondrial membranes, whereas signal peptides direct proteins through the extracellular membrane. By facilitating transport of the protein into compartments inside or outside the cell, these sequences can increase the accumulation of a particular gene product in a particular location. For example, see U.S. Pat. No. 5,258,300.

3′ Sequences: When the expression cassette is to be introduced into a plant cell, the expression cassette can also optionally include 3′ nontranslated plant regulatory DNA sequences that act as a signal to terminate transcription and allow for the polyadenylation of the resultant mRNA. The 3′ nontranslated regulatory DNA sequence preferably includes from about 300 to 1,000 nucleotide base pairs and contains plant transcriptional and translational termination sequences. For example, 3′ elements that can be used include those derived from the nopaline synthase gene of Agrobacterium tumefaciens (Bevan et ah, Nucleic Acid Research. 11:369-385 (1983)), or the terminator sequences for the T7 transcript from the octopine synthase gene of Agrobacterium tumefaciens, and/or the 3′ end of the protease inhibitor I or II genes from potato or tomato. Other 3′ elements known to those of skill in the art can also be employed. These 3′ nontranslated regulatory sequences can be obtained as described in Methods in Enzymology. 153:292 (1987). Many such 3′ nontranslated regulatory sequences are already present in plasmids available from commercial sources such as Clontech, Palo Alto, California. The 3′ nontranslated regulatory sequences can be operably linked to the 3′ terminus of the RFS nucleic acids by standard methods.

Selectable and Screenable Marker Sequences: To improve identification of transformants, a selectable or screenable marker gene can be employed with the expressible RFS nucleic acids. “Marker genes” are genes that impart a distinct phenotype to cells expressing the marker gene and thus allow such transformed cells to be distinguished from cells that do not have the marker. Such genes may encode either a selectable or screenable marker, depending on whether the marker confers a trait which one can ‘select’ for by chemical means, e.g., by use of a selective agent (e.g., an herbicide, antibiotic, or the like), or whether it is simply a trait that one can identify through observation or testing, i.e., by ‘screening’ (e.g., the R-locus trait). Of course, many examples of suitable marker genes are known to the art and can be employed in the practice of the invention.

Included within the terms selectable or screenable marker genes are also genes which encode a “secretable marker” whose secretion can be detected as a means of identifying or selecting for transformed cells. Examples include markers which encode a secretable antigen that can be identified by antibody interaction, or secretable enzymes that can be detected by their catalytic activity. Secretable proteins fall into a number of classes, including small, diffusible proteins detectable, e.g., by ELISA; and proteins that are inserted or trapped in the cell wall (e.g., proteins that include a leader sequence such as that found in the expression unit of extensin or tobacco PR-S).

With regard to selectable secretable markers, the use of a gene that encodes a polypeptide that becomes sequestered in the cell wall, where the polypeptide includes a unique epitope may be advantageous. Such a secreted antigen marker can employ an epitope sequence that would provide low background in plant tissue, a promoter-leader sequence that imparts efficient expression and targeting across the plasma membrane and can produce protein that is bound in the cell wall and yet is accessible to antibodies. A normally secreted wall protein modified to include a unique epitope would satisfy such requirements.

Examples of proteins suitable for modification in this manner include extensin or hydroxyproline rich glycoprotein (HPRG). For example, the maize HPRG (Stiefel et al., The Plant Cell. 2:785-793 (1990)) is well characterized in terms of molecular biology, expression, and protein structure and therefore can readily be employed. However, any one of a variety of extensins and/or glycine-rich wall proteins (Keller et ah, EMBO J. 8:1309-1314 (1989)) could be modified by the addition of an antigenic site to create a screenable marker.

Numerous other possible selectable and/or screenable marker genes will be apparent to those of skill in the art in addition to those forth herein below. Therefore, it will be understood that the discussion herein is exemplary rather than exhaustive. In light of the techniques disclosed herein and the general recombinant techniques that are known in the art, the present invention readily allows the introduction of any gene, including marker genes, into a recipient cell to generate a transformed plant cell, e.g., a monocot cell or dicot cell.

Possible selectable markers for use in connection with the present invention include, but are not limited to, a neo gene (Potrykus et al., Mol. Gen. Genet. 199: 183-188 (1985)) which codes for kanamycin resistance and can be selected for using kanamycin, G418, and the like; a bar gene which codes for bialaphos resistance; a gene which encodes an altered EPSP synthase protein (Hinchee et al., Bio/Technology. 6:915-922 (1988)) thus conferring glyphosate resistance; a nitrilase gene such as bxn from Klebsiella ozaenae which confers resistance to bromoxynil (Stalker et al., Science. 242:419-423 (1988)); a mutant acetolactate synthase gene (ALS) which confers resistance to imidazolinone, sulfonylurea or other ALS-inhibiting chemicals (European Patent Application 154,204 (1985)); a methotrexate-resistant DHFR gene (Thillet et al., J. Biol. Chem. 263:12500-12508 (1988)); a dalapon dehalogenase gene that confers resistance to the herbicide dalapon; or a mutated anthranilate synthase gene that confers resistance to 5-methyl tryptophan. Where a mutant EPSP synthase gene is employed, additional benefit may be realized through the incorporation of a suitable chloroplast transit peptide, CTP (European Patent Application 0 218 571 (1987)).

An illustrative embodiment of a selectable marker gene capable of being used in systems to select transformants is the gene that encode the enzyme phosphinothricin acetyltransferase, such as the bar gene from Streptomyces hygroscopicus or the pat gene from Streptomyces viridochromogenes (U.S. Pat. No. 5,550,318). The enzyme phosphinothricin acetyl transferase (PAT) inactivates the active ingredient in the herbicide bialaphos, phosphinothricin (PPT). PPT inhibits glutamine synthetase, (Murakami et al., Mol. Gen. Genet. 205:42-50 (1986); Twell et al., Plant Physiol. 91:1270-1274 (1989)) causing rapid accumulation of ammonia and cell death. The success in using this selective system in conjunction with monocots was surprising because of the major difficulties that have been reported in transformation of cereals (Potrykus, Trends Biotech. 7:269-273 (1989)).

Screenable markers that may be employed include, but are not limited to, a b-glucuronidase or u id A gene (GUS) that encodes an enzyme for which various chromogenic substrates are known; an R-locus gene, which encodes a product that regulates the production of anthocyanin pigments (red color) in plant tissues (Dellaporta et al., In: Chromosome Structure and Function: Impact of New Concepts, 18* Stadler Genetics Symposium, J. P. Gustafson and R. Appels, eds. (New York: Plenum Press) pp. 263-282 (1988)); a b-lactamase gene (Sutcliffe, Proc. Natl. Acad. Sci. USA. 75:3737-3741 (1978)), which encodes an enzyme for which various chromogenic substrates are known (e.g., PADAC, a chromogenic cephalosporin); a xylE gene (Zukowsky et al., Proc. Natl. Acad. Sci. USA. 80:1101 (1983)) which encodes a catechol dioxygenase that can convert chromogenic catechols; an a-amylase gene (Ikuta et al., Bio/technology 8:241-242 (1990)); a tyrosinase gene (Katz et al., J. Gen. Microbiol. 129:2703-2714 (1983)) which encodes an enzyme capable of oxidizing tyrosine to DOPA and dopaquinone which in turn condenses to form the easily detectable compound melanin; a b-galactosidase gene, which encodes an enzyme for which there are chromogenic substrates; a luciferase (lux) gene (Ow et al., Science. 234:856-859.1986), which allows for bioluminescence detection; or an aequorin gene (Prasher et al., Biochem. Biophys. Res. Comm. 126: 1259-1268 (1985)), which may be employed in calcium-sensitive bioluminescence detection, or a green or yellow fluorescent protein gene (Niedz et al., Plant Cell Reports. 14:403 (1995)).

For example, genes from the maize R gene complex can be used as screenable markers. The R gene complex in maize encodes a protein that acts to regulate the production of anthocyanin pigments in most seed and plant tissue. Maize strains can have one, or as many as four, R alleles that combine to regulate pigmentation in a developmental and tissue specific manner. A gene from the R gene complex does not harm the transformed cells. Thus, an R gene introduced into such cells will cause the expression of a red pigment and, if stably incorporated, can be visually scored as a red sector. If a maize line carries dominant alleles for genes encoding the enzymatic intermediates in the anthocyanin biosynthetic pathway (C2, A1, A2, Bz1 and Bz2), but carries a recessive allele at the R locus, transformation of any cell from that line with R will result in red pigment formation. Exemplary lines include Wisconsin 22 that contains the rg-Stadler allele and TR112, a K55 derivative that is r-g, b, PI. Alternatively any genotype of maize can be utilized if the Cl and R alleles are introduced together.

The R gene regulatory regions may be employed in chimeric constructs to provide mechanisms for controlling the expression of chimeric genes. More diversity of phenotypic expression is known at the R locus than at any other locus (Coe et al., in Corn and Corn Improvement, eds. Sprague, G. F. & Dudley, J. W. (Am. Soc. Agron., Madison, WI), pp. 81-258 (1988)). It is contemplated that regulatory regions obtained from regions 5′ to the structural R gene can be useful in directing the expression of genes, e.g., insect resistance, drought resistance, herbicide tolerance or other protein coding regions. For the purposes of the present invention, it is believed that any of the various R gene family members may be successfully employed (e.g., P, S, Fe, etc.). However, one that can be used is Sn (particularly Sn:bol3). Sn is a dominant member of the R gene complex and is functionally similar to the R and B loci in that Sn controls the tissue specific deposition of anthocyanin pigments in certain seedling and plant cells, therefore, its phenotype is similar to R.

A further screenable marker contemplated for use in the present invention is firefly luciferase, encoded by the lux gene. The presence of the lux gene in transformed cells may be detected using, for example, X-ray film, scintillation counting, fluorescent spectrophotometry, low-light video cameras, photon counting cameras or multiwell luminometry. It is also envisioned that this system may be developed for population screening for bioluminescence, such as on tissue culture plates, or even for whole plant screening.

Other Optional Sequences: An expression cassette of the invention can also further comprise plasmid DNA. Plasmid vectors include additional DNA sequences that provide for easy selection, amplification, and transformation of the expression cassette in prokaryotic and eukaryotic cells. e.g., pUC-derived vectors such as pUC8, pUC9, pUC18, pUC19, pUC23, pUC119, and pUC120, pSK-derived vectors, pGEM-derived vectors, pSP-derived vectors, or pBS-derived vectors. The additional DNA sequences include origins of replication to provide for autonomous replication of the vector, additional selectable marker genes, preferably encoding antibiotic or herbicide resistance, unique multiple cloning sites providing for multiple sites to insert DNA sequences or genes encoded in the expression cassette and sequences that enhance transformation of prokaryotic and eukaryotic cells.

Another vector that is useful for expression in both plant and prokaryotic cells is the binary Ti plasmid (as disclosed in Schilperoort et ah, U.S. Pat. No. 4,940,838) as exemplified by vector pGA582. This binary Ti plasmid vector has been previously characterized by An (Methods in Enzymology. 153:292 (1987)) and is available from Dr. An. This binary Ti vector can be replicated in prokaryotic bacteria such as E. coli and Agrobacterium. The Agrobacterium plasmid vectors can be used to transfer the expression cassette to dicot plant cells, and under certain conditions to monocot cells, such as rice cells. The binary Ti vectors preferably include the nopaline T DNA right and left borders to provide for efficient plant cell transformation, a selectable marker gene, unique multiple cloning sites in the T border regions, the co/El replication of origin and a wide host range replicon. The binary Ti vectors carrying an expression cassette of the invention can be used to transform both prokaryotic and eukaryotic cells but is preferably used to transform dicot plant cells.

In Vitro Screening of Expression Cassettes: Once the expression cassette is constructed and subcloned into a suitable plasmid, it can be screened for the ability to substantially inhibit the translation of an mRNA coding for a seed storage protein by standard methods such as hybrid arrested translation. For example, for hybrid selection or arrested translation, a preselected antisense DNA sequence is subcloned into an SP6/T7 containing plasmids (as supplied by ProMega Corp.). For transformation of plants cells, suitable vectors include plasmids such as described herein. Typically, hybrid arrest translation is an in vitro assay that measures the inhibition of translation of an mRNA encoding a particular seed storage protein. This screening method can also be used to select and identify preselected antisense DNA sequences that inhibit translation of a family or subfamily of zein protein genes. As a control, the corresponding sense expression cassette is introduced into plants and the phenotype assayed.

DNA Delivery of the DNA Molecules into Host Cells: The present invention generally includes steps directed to introducing RFS nucleic acids, such as a preselected cDNA encoding the RFS enzyme, into a recipient cell to create a transformed cell. In some instances, the frequency of occurrence of cells taking up exogenous (foreign) DNA may be low. Moreover, it is most likely that not all recipient cells receiving DNA segments or sequences will result in a transformed cell wherein the DNA is stably integrated into the plant genome and/or expressed. Some may show only initial and transient gene expression. However, certain cells from virtually any dicot or monocot species may be stably transformed, and these cells regenerated into transgenic plants, through the application of the techniques disclosed herein.

Another aspect of the invention is a plant with increased drought tolerance and/or delayed flowering and increased biomass, wherein the plant has an introduced RFS nucleic acid. The plant can be a monocotyledon or a dicotyledon. Another aspect of the invention includes plant cells (e.g., embryonic cells or other cell lines) that can regenerate fertile transgenic plants and/or seeds. The cells can be derived from either monocotyledons or dicotyledons. Suitable examples of plant species include grasses, softwoods, hardwoods, wheat, rice, maize, barley, rye, Brachypodium, Arabidopsis, alfalfa, oats, sorghum, millet, miscanthus, switchgrass, poplar, eucalyptus, sugarcane, bamboo, tobacco, cucumber, tomato, soybean, and the like. In some embodiments, the plant or cell is a monocotyledon plant or cell. For example, the plant or cell can be a grass plant or cell. In some embodiments, the plant or cell is a dicotyledon plant or cell. For example, the plant or cell can be a hardwood plant or cell. The cell(s) may be in a suspension cell culture or may be in an intact plant part, such as an immature embryo, or in a specialized plant tissue, such as callus, such as Type I or Type II callus.

Transformation of the cells of the plant tissue source can be conducted by any one of a number of methods known to those of skill in the art. Examples are:

Transformation by direct DNA transfer into plant cells by electroporation (U.S. Pat. Nos. 5,384,253 and 5,472,869, Dekeyser et ah, The Plant Cell. 2:591-602 (1990)); direct DNA transfer to plant cells by PEG precipitation (Hayashimoto et al. Plant Physiol. 93:857-863 (1990)); direct DNA transfer to plant cells by microprojectile bombardment (McCabe et ah, Bio/Technology. 6:923-926 (1988); Gordon-Kamm et ah. The Plant Cell. 2:603-618 (1990); U.S. Pat. Nos. 5,489,520; 5,538,877; and 5,538,880) and DNA transfer to plant cells via infection with Agrobacterium. Methods such as microprojectile bombardment or electroporation can be carried out with “naked” DNA where the expression cassette may be simply carried on any E. coli-derived plasmid cloning vector. In the case of viral vectors, it is desirable that the system retain replication functions, but lack functions for disease induction.

One method for dicot transformation, for example, involves infection of plant cells with Agrobacterium tumefaciens using the leaf-disk protocol (Horsch et ak, Science 227:1229-1231 (1985). Monocots such as Zea mays can be transformed via microprojectile bombardment of embryogenic callus tissue or immature embryos, or by electroporation following partial enzymatic degradation of the cell wall with a pectinase-containing enzyme (U.S. Pat. Nos. 5,384,253; and 5,472,869). For example, embryogenic cell lines derived from immature Zea mays embryos can be transformed by accelerated particle treatment as described by Gordon-Kamm et al. (The Plant Cell. 2:603-618 (1990)) or U.S. Pat. Nos. 5,489,520; 5,538,877 and 5,538,880, cited above. Excised immature embryos can also be used as the target for transformation prior to tissue culture induction, selection and regeneration as described in U.S. application Ser. No. 08/112,245 and PCT publication WO 95/06128. Furthermore, methods for transformation of monocotyledonous plants utilizing Agrobacterium tumefaciens have been described by Hiei et al. (European Patent 0 604 662, 1994) and Saito et al. (European Patent 0 672 752, 1995).

Methods such as microprojectile bombardment or electroporation are carried out with “naked” DNA where the expression cassette may be simply carried on any E. coli-derived plasmid cloning vector. In the case of viral vectors, it is desirable that the system retain replication functions, but lack functions for disease induction.

The choice of plant tissue source for transformation will depend on the nature of the host plant and the transformation protocol. Useful tissue sources include callus, suspension culture cells, protoplasts, leaf segments, stem segments, tassels, pollen, embryos, hypocotyls, tuber segments, meristematic regions, and the like. The tissue source is selected and transformed so that it retains the ability to regenerate whole, fertile plants following transformation, i.e., contains totipotent cells. Type I or Type II embryonic maize callus and immature embryos are preferred Zea mays tissue sources. Similar tissues can be transformed for softwood or hardwood species. Selection of tissue sources for transformation of monocots is described in detail in U.S. application Ser. No. 08/112,245 and PCT publication WO 95/06128.

The transformation is carried out under conditions directed to the plant tissue of choice. The plant cells or tissue are exposed to the DNA or RNA carrying the RFS nucleic acids for an effective period of time. This may range from a less than one second pulse of electricity for electroporation to a 2-3 days co-cultivation in the presence of plasmid-bearing Agrobacterium cells. Buffers and media used will also vary with the plant tissue source and transformation protocol. Many transformation protocols employ a feeder layer of suspended culture cells (tobacco or Black Mexican Sweet corn, for example) on the surface of solid media plates, separated by a sterile filter paper disk from the plant cells or tissues being transformed.

Electroporation: Where one wishes to introduce DNA by means of electroporation, it is contemplated that the method of Krzyzek et al. (U.S. Pat. No. 5,384,253) may be advantageous. In this method, certain cell wall-degrading enzymes, such as pectin-degrading enzymes, are employed to render the target recipient cells more susceptible to transformation by electroporation than untreated cells. Alternatively, recipient cells can be made more susceptible to transformation, by mechanical wounding.

To effect transformation by electroporation, one may employ either friable tissues such as a suspension cell cultures, or embryogenic callus, or alternatively, one may transform immature embryos or other organized tissues directly. The cell walls of the preselected cells or organs can be partially degraded by exposing them to pectin-degrading enzymes (pectinases or pectolyases) or mechanically wounding them in a controlled manner. Such cells would then be receptive to DNA uptake by electroporation, which may be carried out at this stage, and transformed cells then identified by a suitable selection or screening protocol dependent on the nature of the newly incorporated DNA.

Microprojectile Bombardment: A further advantageous method for delivering transforming DNA segments to plant cells is microprojectile bombardment. In this method, microparticles may be coated with DNA and delivered into cells by a propelling force. Exemplary particles include those comprised of tungsten, gold, platinum, and the like.

It is contemplated that in some instances DNA precipitation onto metal particles would not be necessary for DNA delivery to a recipient cell using microprojectile bombardment. In an illustrative embodiment, non-embryogenic BMS cells were bombarded with intact cells of the bacteria E. coli or Agrobacterium tumefaciens containing plasmids with either the b-glucoronidase or bar gene engineered for expression in maize. Bacteria were inactivated by ethanol dehydration prior to bombardment. A low level of transient expression of the b-glucoronidase gene was observed 24-48 hours following DNA delivery. In addition, stable transformants containing the bar gene were recovered following bombardment with either E. coli or Agrobacterium tumefaciens cells. It is contemplated that particles may contain DNA rather than be coated with DNA. Hence it is proposed that particles may increase the level of DNA delivery but are not, in and of themselves, necessary to introduce DNA into plant cells.

The microprojectile bombardment is an effective means of reproducibly stably transforming monocots that avoids the need to prepare and isolate protoplasts (Christou et al., PNAS. 84:3962-3966 (1987)), avoids the formation of partially degraded cells, and the susceptibility to Agrobacterium infection is not required. An illustrative embodiment of a method for delivering DNA into maize cells by acceleration is a Biolistics Particle Delivery System, which can be used to propel particles coated with DNA or cells through a screen, such as a stainless steel or Nytex screen, onto a filter surface covered with maize cells cultured in suspension (Gordon-Kamm et al., The Plant Cell. 2:603-618 (1990)). The screen disperses the particles so that they are not delivered to the recipient cells in large aggregates. It is believed that a screen intervening between the projectile apparatus and the cells to be bombarded reduces the size of projectile aggregate and may contribute to a higher frequency of transformation, by reducing damage inflicted on the recipient cells by an aggregated projectile.

For bombardment, cells in suspension are preferably concentrated on filters or solid culture medium. Alternatively, immature embryos or other target cells may be arranged on solid culture medium. The cells to be bombarded are positioned at an appropriate distance below the macroprojectile stopping plate. If desired, one or more screens are also positioned between the acceleration device and the cells to be bombarded. Using techniques set forth herein, one may obtain up to 1000 or more foci of cells transiently expressing a marker gene. The number of cells in a focus which express the exogenous gene product 48 hours post-bombardment often range from about 1 to 10 and average about 1 to 3.

In bombardment transformation, one may optimize the prebombardment culturing conditions and the bombardment parameters to yield the maximum numbers of stable transformants. Both the physical and biological parameters for bombardment can influence transformation frequency. Physical factors are those that involve manipulating the DNA/microprojectile precipitate or those that affect the path and velocity of either the macro- or microprojectiles. Biological factors include all steps involved in manipulation of cells before and immediately after bombardment, the osmotic adjustment of target cells to help alleviate the trauma associated with bombardment, and also the nature of the transforming DNA, such as linearized DNA or intact supercoiled plasmid DNA.

One may wish to adjust various bombardment parameters in small scale studies to fully optimize the conditions and/or to adjust physical parameters such as gap distance, flight distance, tissue distance, and helium pressure. One may also minimize the trauma reduction factors (TRFs) by modifying conditions which influence the physiological state of the recipient cells and which may therefore influence transformation and integration efficiencies. For example, the osmotic state, tissue hydration and the subculture stage or cell cycle of the recipient cells may be adjusted for optimum transformation. Execution of such routine adjustments will be known to those of skill in the art.

An Example of Production and Characterization of Stable Transgenic Plants: After effecting delivery of a RFS nucleic acid to recipient cells by any of the methods discussed above, the transformed cells can be identified for further culturing and plant regeneration. As mentioned above, to improve the ability to identify transformants, one may desire to employ a selectable or screenable marker gene as, or in addition to, the expressible RFS nucleic acids. In this case, one would then generally assay the potentially transformed cell population by exposing the cells to a selective agent or agents, or one would screen the cells for the desired marker gene trait.

Selection: An exemplary embodiment of methods for identifying transformed cells involves exposing the bombarded cultures to a selective agent, such as a metabolic inhibitor, an antibiotic, herbicide or the like. Cells which have been transformed and have stably integrated a marker gene conferring resistance to the selective agent used, will grow and divide in culture. Sensitive cells will not be amenable to further culturing.

To use the har-bialaphos or the EPSPS-glyphosate selective system, bombarded tissue is cultured for about 0-28 days on nonselective medium and subsequently transferred to medium containing from about 1-3 mg/l bialaphos or about 1-3 mM glyphosate, as appropriate. While ranges of about 1-3 mg/l bialaphos or about 1-3 mM glyphosate can be employed, it is proposed that ranges of at least about 0.1-50 mg/l bialaphos or at least about 0.1-50 mM glyphosate will find utility in the practice of the invention. Tissue can be placed on any porous, inert, solid or semi solid support for bombardment, including but not limited to filters and solid culture medium. Bialaphos and glyphosate are provided as examples of agents suitable for selection of transformants, but the technique of this invention is not limited to them.

An example of a screenable marker trait is the red pigment produced under the control of the R-locus in maize. This pigment may be detected by culturing cells on a solid support containing nutrient media capable of supporting growth at this stage and selecting cells from colonies (visible aggregates of cells) that are pigmented. These cells may be cultured further, either in suspension or on solid media. The R-locus is useful for selection of transformants from bombarded immature embryos. In a similar fashion, the introduction of the Cl and B genes will result in pigmented cells and/or tissues.

The enzyme luciferase is also useful as a screenable marker in the context of the present invention. In the presence of the substrate luciferin, cells expressing luciferase emit light which can be detected on photographic or X-ray film, in a luminometer (or liquid scintillation counter), by devices that enhance night vision, or by a highly light sensitive video camera, such as a photon counting camera. All of these assays are nondestructive and transformed cells may be cultured further following identification. The photon counting camera is especially valuable as it allows one to identify specific cells or groups of cells which are expressing luciferase and manipulate those in real time.

It is further contemplated that combinations of screenable and selectable markers may be useful for identification of transformed cells. For example, selection with a growth inhibiting compound, such as bialaphos or glyphosate at concentrations below those providing 100% inhibition followed by screening of growing tissue for expression of a screenable marker gene such as luciferase would allow one to recover transformants from cell or tissue types that are not amenable to selection alone. In an illustrative embodiment embryogenic Type II callus of Zea mays L. can be selected with sub-lethal levels of bialaphos. Slowly growing tissue was subsequently screened for expression of the luciferase gene and transformants can be identified.

Regeneration and Seed Production: Cells that survive the exposure to the selective agent, or cells that have been scored positive in a screening assay, are cultured in media that supports regeneration of plants. One example of a growth regulator that can be used for such purposes is dicamba or 2,4-D. However, other growth regulators may be employed, including NAA, NAA+2,4-D or perhaps even picloram. Media improvement in these and like ways can facilitate the growth of cells at specific developmental stages. Tissue can be maintained on a basic media with growth regulators until sufficient tissue is available to begin plant regeneration efforts, or following repeated rounds of manual selection, until the morphology of the tissue is suitable for regeneration, at least two weeks, then transferred to media conducive to maturation of embryoids. Cultures are typically transferred every two weeks on this medium. Shoot development signals the time to transfer to medium lacking growth regulators.

The transformed cells, identified by selection or screening and cultured in an appropriate medium that supports regeneration, can then be allowed to mature into plants. Developing plantlets are transferred to soilless plant growth mix, and hardened, e.g., in an environmentally controlled chamber at about 85% relative humidity, about 600 ppm C02, and at about 25-250 microeinsteins/sec-m² of light. Plants can be matured either in a growth chamber or greenhouse. Plants are regenerated from about 6 weeks to 10 months after a transformant is identified, depending on the initial tissue. During regeneration, cells are grown on solid media in tissue culture vessels. Illustrative embodiments of such vessels are petri dishes and Plant Con™. Regenerating plants can be grown at about 19° C. to 28° C. After the regenerating plants have reached the stage of shoot and root development, they may be transferred to a greenhouse for further growth and testing.

Mature plants are then obtained from cell lines that are known to express the trait. In some embodiments, the regenerated plants are self-pollinated. In addition, pollen obtained from the regenerated plants can be crossed to seed grown plants of agronomically important inbred lines. In some cases, pollen from plants of these inbred lines is used to pollinate regenerated plants. The trait is genetically characterized by evaluating the segregation of the trait in first and later generation progeny. The heritability and expression in plants of traits selected in tissue culture are of interest if the traits are to be commercially useful.

Regenerated plants can be repeatedly crossed to inbred plants to introgress the RFS nucleic acids into the genome of the inbred plants. This process is referred to as backcross conversion. When a sufficient number of crosses to the recurrent inbred parent have been completed to produce a product of the backcross conversion process that is substantially isogenic with the recurrent inbred parent except for the presence of the introduced RFS nucleic acids, the plant is self-pollinated at least once to produce a homozygous backcross converted inbred containing the RFS nucleic acids. Progeny of these plants are true breeding.

Alternatively, seed from transformed monocot plants regenerated from transformed tissue cultures is grown in the field and self-pollinated to generate true breeding plants.

Seed from the fertile transgenic plants can then be evaluated for the presence and/or expression of the RFS nucleic acids (or RFS proteins). Transgenic plant and/or seed tissue can be analyzed for RFS expression using standard methods such as SDS polyacrylamide gel electrophoresis, liquid chromatography (e.g., HPLC) or other means of detecting a product of RFS activity (e.g., increased glucan content and/or good growth).

Once a transgenic seed expressing the RFS sequence and having an increase in glucan content in the plant is identified, the seed can be used to develop true breeding plants. The true breeding plants are used to develop a line of plants with an increase in the percent of glucan content and growth of the plant while still maintaining other desirable functional agronomic traits. Adding the trait of increased glucan content and growth and normal to improved growth of the plant can be accomplished by back-crossing with this trait and with plants that do not exhibit this trait and studying the pattern of inheritance in segregating generations. Those plants expressing the target trait in a dominant fashion are preferably selected.

Back-crossing is carried out by crossing the original fertile transgenic plants with a plant from an inbred line exhibiting desirable functional agronomic characteristics while not necessarily expressing the trait of an increased percent of glucan synthase activity, normal to improved growth, and/or protein folding in the plant. The resulting progeny are then crossed back to the parent that expresses the increased RFS trait (more glucans, normal to improved growth, and/or protein folding). The progeny from this cross will also segregate so that some of the progeny carry the trait and some do not. This back-crossing is repeated until an inbred line with the desirable functional agronomic traits, and with expression of the trait involving an increase in glucan content and normal to improved growth of the plant. Such expression of the increased glucan content and/or normal to improved growth of plant can be expressed in a dominant fashion.

Subsequent to back-crossing, the new transgenic plants can be evaluated for an increase in the weight percent of glucan synthase activity, normal to improved growth, and/or protein folding of the plant. This can be done, for example, by immunofluorescence analysis of whole plant cell walls (e.g., by microscopy), glucan synthase activity assays, protein folding assays, growth measurements, and any of the assays described herein or available to those of skill in the art.

The new transgenic plants can also be evaluated for a battery of functional agronomic characteristics such as lodging, kernel hardness, yield, resistance to disease, resistance to insect pests, drought resistance, and/or herbicide resistance.

As described herein, expression of RFS can not only increase the tolerance of plant tissues to drought, but such expression can also delay flowering time with concomitant increase in plant biomass. Hence it is useful to modify a variety of plant types to express RFS.

Plants that can be improved include but are not limited to forage plants (e.g., alfalfa, clover, soybeans, turnips, bromegrass, bluestem, and fescue), starch plants (e.g., canola, potatoes, lupins, sunflower and cottonseed), grains (maize, wheat, barley, oats, rice, sorghum, millet and rye), grasses (switchgrass, prairie grass, wheat grass, sudangrass, sorghum, straw-producing plants, miscanthus, switchgrass), sugar producing plants (sugarcane, beets), vegetable plants (e.g., cucumber, tomato), Brachypodium, Arabidopsis, bamboo, softwood, hardwood and other woody plants (e.g., those used for paper production such as poplar species, pine species, and eucalyptus). In some embodiments the plant is a forage crop species or a species useful for production of biofuels. Examples of plants useful for pulp and paper production include most pine species such as loblolly pine, Jack pine. Southern pine. Radiata pine, spruce, Douglas fir and others. Hardwoods that can be modified as described herein include aspen, poplar, eucalyptus, and others. Plants useful for making biofuels and ethanol include corn, Brachypodium, grasses (e.g., miscanthus, switchgrass, and the like), as well as trees such as poplar, aspen, willow, and the like. Plants useful for generating dairy forage include legumes such as alfalfa, as well as clover, soybeans, turnips, Brachypodium, Arabidopsis, and forage grasses such as bromegrass, and bluestem.

Determination of Stably Transformed Plant Tissues: To confirm the presence of the RFS nucleic acids in the regenerating plants, or seeds or progeny derived from the regenerated plant, a variety of assays may be performed. Such assays include, for example, molecular biological assays available to those of skill in the art, such as Southern and Northern blotting and PCR; biochemical assays, such as detecting the presence of a protein product, e.g., by immunological means (ELISAs and Western blots) or by enzymatic function; plant part assays, such as leaf, seed or root assays; and also, by analyzing the phenotype of the whole regenerated plant.

Whereas DNA analysis techniques may be conducted using DNA isolated from any part of a plant, RNA may only be expressed in particular cells or tissue types and so RNA for analysis can be obtained from those tissues. PCR techniques may also be used for detection and quantification of RNA produced from introduced RFS nucleic acids. PCR also be used to reverse transcribe RNA into DNA, using enzymes such as reverse transcriptase, and then this DNA can be amplified by use of conventional PCR techniques. Further information about the nature of the RNA product may be obtained by Northern blotting. This technique will demonstrate the presence of an RNA species and give information about the integrity of that RNA. The presence or absence of an RNA species can also be determined using dot or slot blot Northern hybridizations. These techniques are modifications of Northern blotting and also demonstrate the presence or absence of an RNA species.

While Southern blotting and PCR may be used to detect the RFS nucleic acid in question, they do not provide information as to whether the preselected DNA segment is being expressed. Expression may be evaluated by specifically identifying the protein products of the introduced RFS nucleic acids or evaluating the phenotypic changes brought about by their expression.

Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange, liquid chromatography or gel exclusion chromatography. The unique structures of individual proteins offer opportunities for use of specific antibodies to detect their presence in formats such as an ELISA assay. Combinations of approaches may be employed with even greater specificity such as Western blotting in which antibodies are used to locate individual gene products that have been separated by electrophoretic techniques. Additional techniques may be employed to absolutely confirm the identity of the RFS such as evaluation by amino acid sequencing following purification. The Examples of this application also provide assay procedures for detecting and quantifying RFS activity. Other procedures may be additionally used.

The expression of a gene product can also be determined by evaluating the phenotypic results of its expression. These assays also may take many forms including but not limited to analyzing changes in the chemical composition, morphology, or physiological properties of the plant. Chemical composition may be altered by expression of preselected DNA segments encoding storage proteins which change amino acid composition and may be detected by amino acid analysis.

Release of Fermentable Sugars from Plant Biomass

Plant parts, components and biomass from plants expressing RFS can be converted into fermentable sugars using various procedures. For example, the plant parts, components and biomass from plants expressing RFS can be dried and/or ground up so that the polysaccharides become accessible to enzymatic cleavage.

Effective enzyme mixtures for biomass deconstruction can have combined catalytic activities so that the enzymes can cleave substantially all saccharide linkages found in plant cell walls to release free, fermentable sugar residues. Such enzyme mixtures can often be derived from microorganisms. Many microorganisms that live in lignocellulose-rich environments secrete large numbers and broad ranges of cell wall-active enzymes, including, but not limited to, cellulases, hemicellulases, pectinases, and/or proteases. Most commercially available deconstruction enzyme mixtures contain between approximately twenty-five to one hundred and fifty (25-150) enzymes. Nagendran et al., Fung. Genet. Biol. 46: 427-435 (2009); Banerjee et al., Bioresour. Technol. 101: 9097-9105 (2010); and Scott-Craig et al., J Biol Chem 286:42848-42854 (2011). For example, commercial enzyme mixtures can be used that include hemicellulose degrading enzymes such as b-14-xylanasc. b-xylosidase, a-arabinosidase, mixed-linked glucanase, a-glucuronidase, etc. Examples of commercial enzyme mixtures that can be employed to release fermentable sugars from plant biomass include Spezyme CP, Accellerase® 1000, Multifect Xylanase, Cellic® CTec2, HTec2, CTec3, HTec3, and AlternaFuel® CMAX.

Incubation of the plant biomass with the enzyme mixture can be performed at a temperature ranging from approximately 400 to approximately 60° C. In one embodiment, the incubation is performed at a pH ranging from approximately 4 to approximately 6.

### Definitions

As used herein, the term “plant” is used in its broadest sense. It includes, but is not limited to, any species of grass (e.g. forage, grain-producing, turf grass species), ornamental or decorative, crop or cereal, fodder or forage, fruit or vegetable, fruit plant or vegetable plant, herb plant, woody plant, flower plant or tree. It is not meant to limit a plant to any particular structure. It also refers to a unicellular plant (e.g. microalga) and a plurality of plant cells that are largely differentiated into a colony (e.g. volvox) or a structure that is present at any stage of a plant's development. Such structures include, but are not limited to, a seed, a tiller, a sprig, a stolen, a plug, a rhizome, a shoot, a stem, a leaf, a flower petal, a fruit, et cetera.

As used herein, “isolated” means a nucleic acid or polypeptide has been removed from its natural or native cell. Thus, the nucleic acid or polypeptide can be physically isolated from the cell or the nucleic acid or polypeptide can be present or maintained in another cell where it is not naturally present or synthesized.

The term “transgenic” when used in reference to a plant or leaf or fruit or seed or plant biomass, for example a “transgenic plant,” transgenic leaf,” “transgenic fruit,” “transgenic fruit,” “transgenic seed,” “transgenic biomass.” or a “transgenic host cell” refers to a plant or leaf or fruit or seed or biomass that contains at least one heterologous or foreign gene in one or more of its cells. The term “transgenic plant material” refers broadly to a plant, a plant structure, a plant tissue, a plant seed or a plant cell that contains at least one heterologous gene in one or more of its cells.

The term “transgene” refers to a foreign gene that is placed into an organism (e.g. a plant) or host cell by the process of transfection. The term “foreign gene” or heterologous gene refers to any nucleic acid (e.g., gene sequence) that is introduced into the genome of an organism or tissue of an organism or a host cell by experimental manipulations, such as those described herein, and may include gene sequences found in that organism so long as the introduced gene does not reside in the same location, as does the naturally occurring gene.

As used herein, the term “heterologous” when used in reference to a nucleic acid or protein refers to a nucleic acid or protein that has been manipulated in some way. For example, a heterologous nucleic acid includes a nucleic acid from one species introduced into another species. A heterologous nucleic acid also includes a nucleic acid that is native to an organism that has been altered in some way (e.g., mutated, added in multiple copies, present in a locus within the genome, expressed from an autonomously replicating vector, linked to a non-native promoter, linked to a mutated promoter, or linked to an enhancer sequence, etc.). Heterologous nucleic acids may comprise plant gene sequences that comprise cDNA forms of a plant gene; the cDNA sequences may be expressed in either a sense (to produce mRNA) or anti-sense orientation (to produce an anti-sense RNA transcript that is complementary to the mRNA transcript). In some cases, heterologous nucleic acids are distinguished from endogenous plant genes in that the heterologous nucleic acids can be joined to nucleotide sequences comprising regulatory elements such as promoters that are not found naturally associated with the nucleic acid. In another example, the heterologous nucleic acids are associated with portions of the chromosome not found in nature (e.g., genes expressed in loci where the gene is not normally expressed).

As used herein, a “native” nucleic acid or polypeptide means a DNA, RNA or amino acid sequence or segment that has not been manipulated in vitro, i.e., has not been isolated, purified, and/or amplified.

As used herein, the term “wild-type” when made in reference to a gene refers to a functional gene common throughout an outbred population. As used herein, the term “wild-type” when made in reference to a gene product refers to a functional gene product common throughout an outbred population. A functional wild-type gene is that which is most frequently observed in a population and is thus arbitrarily designated the “normal” or “wild-type” form of the gene. As used herein, the term “wild-type” when made in reference to a plant refers to the plant type common throughout an outbred population that has not been genetically manipulated to contain an expression cassette, e.g., any of the expression cassettes described herein.

The following non-limiting Examples illustrate how aspects of the technology have been developed and can be made and used. Additional embodiments of the disclosure reside in specific examples and data described in more detail herein.

### Example 1: Materials and Methods

This example describes some of the materials and methods used in developing the technology.

### Plant Material, Growth and Treatment:

A. thaliana (ecotype Col-0) seeds were sterilized and planted as described in (Ying et al., 2022). A. thaliana bolting time was monitored daily and determined as the number of days from sowing to the first elongation of the floral stem at 1 mm height. The T-DNA insertion mutant line of At3g43110 (atrfs1, GK-422C11) and At5g20790 (atrfs2, SALK_030486) were obtained from GABI-Kat (Kleinboelting et al., 2012) or Arabidopsis Biological Resource Center (ABRC), respectively. Homozygous mutant plants were identified or validated according to the of SiGnAL database PCR-based protocol (http//signal.salk.edu/).

B. distachyon (ecotype BD21-3) seeds were surface sterilized by solution containing 30% (v/v) bleach and 0.05% (v/v) Tween-20, then stratified for 3 days in the dark at 4° C., and germinated on ½ MS agar (1% w/v) plates. Plants were cultured vertically in growth chamber (175 μmol² s⁻¹ light intensity, 22° C./18° C., 16-h-light and 8-h-dark cycle) for 7 days, then transplanted into soil (SureMix) and grown in greenhouse (24° C., 16-h-light and 8-h-dark cycle) until plants were matured. Leaf samples of mature plants were harvested for phenotypic, biochemical or molecular examination. Leaf thickness was measured by using digital micrometer. For P-limitation treatment, sterilized seeds were directly placed on ½ MS agar containing 675 μM or 0 μM phosphate and grown vertically in growth chamber for 10 days. For dehydration treatment, three-week-old seedlings were taken immediately out from pots and placed on the Whatman 3MM paper to dry in ambient temperature for 6 hr. For freezing treatment, the seedlings at the same development stage were transferred to a growth chamber at −4° C. for 6 hr. For phytohormone treatments, 100 μM of abscisic acid (ABA) or methyl jasmonate (MeJA) was sprayed on the leaves of 3-week-old BD21-3 seedlings and covered with plastic wrap for 30 min. For tunicamycin (Tm) treatment, 7-day-old BD21-3 seedlings were transferred to ½ MS agar supplemented with 50 ng mL⁻¹ Tm and grown vertically in growth chamber for 3 hr. Target tissues were sampled at indicated time point, quickly frozen in liquid N₂ and stored in −80° C. for further studies.

### Quantitative Reverse Transcription PCR (RT-qPCR):

Total RNA was extracted using the RNeasy® plant mini kit (Qiagen). Complementary DNA (cDNA) was synthesized from 1 μg of DNasc treated RNA using SuperScript™ III reverse transcriptase (Invitrogen). Quantitative reverse transcription PCR (RT-qPCR) was conducted as described in (Ying et al., 2022). The A. thaliana GAPDH (At1g13440) or B. distachyon UBC18 (Bd4g00660) gene was used as an internal control (Czechowski e al., 2005; Hong et al., 2008). All the experiments were repeated at least three times using cDNAs prepared from two different biological replicates with representative results shown in the figures. Sequences of primers used in the study are listed in Table 2:

TABLE 1
Primer Sequences

| SEQ | | | | |
|---|---|---|---|---|
| ID NO | GeneID | Primer name | Sequence (5′ to 3′) | Purpose |
| 37 | At3g43110 | AtTM1-FP | GGAGACTGAGGATTGGAGTTTG | RT-qPCR |
| 38 | (AtRFS1) | AtTM1-RP | TCTCTCCGTCATCTTCTTCTCT | analysis |
| 39 | At5g20790 | AtTM2-FP | ATCTTGAGAAGCTTTCTTGGTTTG | |
| 40 | (AtRFS2) | AtTM2-RP | GCGTTCCTTCTTGGTCTCTT | |
| 41 | Bd4g31140 | BdiTM-FP | CCTCGTGGACAACAAGAAGATA | |
| 42 | (BdRFS) | BdiTM-RP | AGCTCGGCTCGCTAATTG | |
| 43 | At1g13440 | GAPDH-FP | TTGGTGACAACAGGTCAAGCA | |
| 44 | (GAPDH) | GAPDH-RP | AAACTTGTCGCTCAATGCAATC | |
| 45 | Bd4g00660 | UBC18-FP | GGAGGCACCTCAGGTCATTT | |
| 46 | (BdUBC18) | UBC18-RP | ATAGCGGTCATTGTCTTGCG | |
| 47 | Bd2g06050 | BIP2-FP | AGAACCCGGAAGCGGATAAG | |
| 48 | (BdBiP2) | BIP2-RP | GAAGACAGGGTTGCAGAGGT | |
| 49 | Bd3g34200 | MYC2-FP | TACTCTCAGGACCAGCTCAA | |
| 50 | (BdMYC2) | MYCZ-RP | CAAGATCACAGTTCCCTCACTATC | |
| 51 | Bd1g11670 | LOX-FP | CGTCCATTGTACTCCTGCTTAC | |
| 52 | (BdLOX) | LOX-RP | GCGTACAAGCTGATCTCACA | |
| 53 | Bd3g23190 | JAZ-FP | ACCTCCACCTCACTTCTTCT | |
| 54 | (BdJAZ) | JAZ-RP | CTGCGGCCATTACTTCTTCT | |
| 55 | Bd3g37650 | OPR3-FP | AAGGTGTAGTTTCAGCCATAGGA | |
| 56 | (BdOPR3) | OPR3-RP | GATAGTGGGTCAGAATCAGTTGC | |
| 57 | Bd1g48830 | FT1-FP | CACACTACACACACGCAAGTACTGT | |
| 58 | (BdFT1) | FT1-RP | CAGCACGTCCCCCACAA | |
| 59 | Bd2g07070 | FT2-FP | TGGTTGTGATGGTCCGTTTG | |
| 60 | (BdFT2) | FT2-RP | AGACAGAACCGACTTGCTAGAAATTAC | |
| 61 | At3g43110 | AtTM1 PRO- | CACCGCCTTCCCGTCACTGTC | Promoter |
| | (AtRFS1) | FP | | cloning |
| 62 | | AtTM1 PRO- | CGAAGAAGATTGAATCAGTGGTTAT | |
| | | RP | | |
| 63 | At5g20790 | AtTM2 PRO- | CACCTTGCTGGTCTCGGGAATTG | |
| | (AtRFS2) | FP | | |
| 64 | | AtTM2 PRO- | GGTGAACGATTCAGTCGGTTAT | |
| | | RP | | |
| 65 | At3g43110 | AtTM1 CDS- | CACCATGGCGACTTTGGATTCTC | CDS cloning |
| | (AtRFS1) | FP | | |
| 66 | | AtTM1 CDS- | TCAACCATAACACTCCTTA | |
| | | RP | | |
| 67 | At5g20790 | AtTM2 CDS- | CACCATGTCGACTTTGGAATCT | |
| | (AtRFS2) | FP | | |
| 68 | | AtTM2 CDS- | TTAGACACATGCCGCCGT | |
| | | RP | | |
| 69 | Bd4g31140 | BdiTM CDS-FP | CACCATGGAGCTCCTGGACGTGG | |
| 70 | (BdRFS) | BdiTM CDS-RP | CTATACTGACAAGTGCAGT | |
| | T-DNA insertion | | | |
| | mutant | Primer name | Sequence (5′ to 3′) | Purpose |
| 71 | At3g43110 (GK- | tm1-LP | ATGACATACACGAAAACTAC | T-DNA |
| 72 | 422C11) | tm1-RP | GTTAGAGGTTTTGGCTTTCGACTA | mutant |
| 73 | At5g20790 | tm2-LP | CGACCGCACTTGTAAGCTTAG | genotyping |
| 74 | (SALK_030486) | tm2-RP | CGTCAACGTCTACCTCGTAGC | |

### Identification and Phylogenetic Analysis of RFS Proteins:

To search RFS proteins from different plant species, a BLASTP search was conducted on the Phytozome v13 (Goodstein et al., 2011) website using the C-terminal conserved domain of At3g43110 protein as query sequence. Identified protein sequences with low E value (<0.001) were downloaded and aligned using the MUSCLE algorithm with the MEGA X software (Kumar et al., 2018). A phylogenetic rooted tree was constructed with MEGA X software by using the UPGMA algorithm with the default settings. Bootstrapping was performed 1000 times. The inferred trees were visualized using iTOL (Letunic and Bork, 2021).

### Generation of Transformants:

For histochemical GUS analysis of A. thaliana RFS genes, 1.561-bp or 486-bp upstream (from start codon) fragment of At3g43110 or At5g20790 gene was amplified from genomic DNA and subsequently cloned into the vector pBGWFS7 (Karimi et al., 2002). For generation of CRISPR knockout (CRISPR-ko) mutants, two target sgRNA sequences of Bd4g31140 gene were designed by CRISPR-P 2.0 online program (Liu et al., 2017) and constructed into pRGEB32 vector as described previously (Xie et al., 2015). For generation of overexpressor lines, full-length coding region of Bd4g31140 gene was amplified from B. distachyon BD21-3 seedling cDNA and ligated to pANIC6B vector (Mann et al., 2012). Transformation procedures for A. thaliana or B. distachyon were described in (Ying et al., 2022).

Histochemical GUS (β-glucuronidase) staining:
The histochemical GUS staining was performed as previously described (Ying et al., 2022). Briefly, seedlings were incubated in a GUS staining solution containing 100 mM sodium phosphate (pH 7.0), 1 mM EDTA, 0.05% Triton X-100, 1 mM potassium ferricyanide/ferrocyanide, and 0.5 mg mL⁻¹ X-glucuronide (Goldbio) at 37° C. for 1 to 3 hr. Then samples were progressively cleared in gradient ethanol solution for 30 min. Images were acquired using a Nikon SMZ1500 stereomicroscope.

### Tobacco Infiltration and Fluorescence Microscopy:

The full-length coding region of At3g43110 gene were ligated into pEarlyGate103 vector (Earley et al., 2006), to generate GFP-fused protein driven by CaMV35S promoter. Constructs were transformed into N. benthamiana using Agro-infiltration (Li, 2011). Forty-eight hours after transformation, GFP fluorescence was monitored with a Leica TCS SP8 confocal laser-scanning microscope (Ex: 488 nm; Em 507 nm). All experiments have been repeated at least three times and representative images are displayed in figures.

### Transmission Electron Microscopy (TEM):

The middle third of fully expanded leaves from 3-week-old B. distachyon plants were collected for TEM analysis. Samples (1 mm² size) were fixed, stained and sectioned as described in (Lundquist et al., 2013). The images were captured by a JEOL 1400 Flash Transmission Electron Microscopy. Measurements of plastoglobule densities in chloroplast areas were made using ImageJ.

### Extraction and Quantification of Jasmonic Acid (JA) and its Derivatives:

Leaf tissues were collected at the indicated developmental stages and immediately snap-frozen in liquid N₂. JAs were extracted and quantified as previously described (Havko et al., 2020). Briefly, frozen tissue was ground by TissueLyser (QIAGEN) and extracted in cold buffer containing 80% (v/v) methanol (Millipore Sigma), 0.1% (v/v) formic acid and 0.1 mg mL⁻¹ butylated hydroxytoluene (BHT). (+)-Abscisic Acid-d₆ (Cayman Chemical) was used as internal standard. Measurements were performed by using a Xevo TQ-XS UPLC/MS/MS.

### Lipid Analyses:

Total lipid was extracted from leaf tissues of different genotypes at the same developmental stage. The procedures of extraction, thin-layer chromatography (TLC) of polar lipids, transesterification, and gas-liquid chromatography (GC) were performed as described previously (Wang and Benning, 2011; Yang et al., 2017). Briefly, lipid separation was performed by activated (NH₄)₂SO₄-impregnated silica gel TLC plates (TLC Silica gel 60, EMD Chemical) with a solvent consisting of acetone, toluene, and water (91:30:7.5 by volume). Lipids were visualized by brief exposure to iodine vapor on TLC plates, scraped and converted to methyl esters, which were subsequently quantified by Agilent 7890A GC system.

For prenyl-lipid analysis, the extraction was conducted according to previous report (Espinoza-Corral et al., 2021). Compounds were separated by using Nexera-i2040C-3D HPLC system (Shimadzu) and the concentrations were determined by using LabSolutions software (Version 5.97 SP1, Shimadzu), based on calibration curves. All the experiments were repeated three times by using different biological replicates.

Measurement of Total Chlorophyll Contents. Malondialdehyde and Relative Water Content:

For total chlorophyll measurements, 100 mg of ground leaf tissue was incubated with 10 mL of 80% acetone (v/v) at 80° C. for 20 min. The absorbance of chlorophyll extraction was detected with spectrophotometer (Biotek) at 645- and 663-nm wavelength separately, and the chlorophyll content was calculated using the following formula: total chlorophyll content (mg g⁻¹ fresh weight)=[20.2×(A645)+8.02×(A663)]×volume/fresh weight. The content of malondialdehyde (MDA) in the leaf tissue was measured as described in (DHINDSA et al., 1981). Briefly, approximately 100 mg of leaf sample was homogenized in 0.8 mL 0.1% (w/v) trichloroacetic acid (TCA). The homogenate was centrifuged at 10,000 g for 5 min. About 0.4 mL the supernatant was mixed with 1.6 mL 20% (w/v) TCA containing 0.5% (w/v) thiobarbituric acid (TBA). The mixture was heated at 95° C. for 30 min and then quickly chilled on ice. After centrifuging at 10,000 g for 10 min, the absorbance of the supernatant was respectively read at 532 nm and 600 nm (non-specific absorption) by using spectrophotometer. The content of MDA (μM mg⁻¹ fresh weight) was calculated as: (A532−A600)×2/(155×0.1). For leaf relative water content (RWC) analysis, four individual leaves from the same plant were collected together as one replicate. The RWC (%) was calculated as: 100×(fresh weight−dry weight)/(turgid weight−dry weight). All the experiments were repeated three times using samples harvested from five biological replicates of each genotype.

Detection of Hydrogen Peroxide (H₂O₂):

To visually detect endogenous H₂O₂, B. distachyon fully-expanded leaves at same developmental stage were collected and incubated in 3,3′-Diaminobenzidine (DAB, 1 mg mL⁻¹, Millipore Sigma) solution for 24 hr at room temperature. Leave samples were then decolorized in boiling ethanol for 30 min. Images were captured by an Epson Perfection V700 scanner. All the experiments were repeated three times using samples harvested from five biological replicates of each genotype.

### Co-Immunoprecipitation (Co-IP) Analysis:

For heterologous expression of BdRFS, the full-length (FL) and N-terminally truncated (Δ1-45, residues 1 to 45, including the transmembrane domain) cDNA fragments were subcloned into a modified pET28b vector (New England Biolabs) carrying an N-terminal 6×His and SUMO(Small Ubiquitin-like Modifier) tag. For recombinant protein production, constructs were separately introduced into the Escherichia coli BL21 (DE3) pLysS competent cells (Agilent). The purification was performed using Ni-NTA agarose (Qiagen, Hilden, Germany) according to the manufacturer's instructions. Protein concentration was quantified using Pierce™ Rapid Gold BCA Protein Assay kit (Thermo Fisher Scientific), and stored in −80° C. freezer for further study.

Co-IPs and crude protein extractions were carried out according to Ying, et al. (Ying et al., 2022) with minor modifications. Briefly, recombinant His-SUMO-BdRFS^{Δ1-45} or His-SUMO protein (2 μg) were incubated with 25 μL Dynabeads™ His tag magnetic beads (Invitrogen) in the buffer containing 50 mM Sodium Phosphate (pH 8.0), 300 mM NaCl, and 0.01% (v/v) Tween™-20 for 10 min at 4° C. Then, the supernatant was discarded by placing the tube on a magnet for 2 min. After three times washing with the same buffer, crude protein extract (10 mg) was added to the magnetic beads-recombinant protein complex and incubated on a rotator for 1 hr at 4° C. The bound protein was eluted by using 100 μL Elution buffer containing 50 mM Sodium Phosphate (pH 8.0), 300 mM NaCl, 0.01% (v/v) Tween-20, and 300 mM Imidazole. Composition of the eluates and inputs were determined using LC-MS/MS as follows: proteins were loaded onto a 12.5% pre-cast BioRad Criterion 1D gel and electrophoresed at 50V constant for ˜20 min or until the dye front migrated 2-3 mm below the well. Electrophoresis was stopped and the gel stained using Coomassie Blue. Concentrated sample bands were then excised from the gel and placed into individual microfuge tubes. Gel bands were digested in-gel according to (Shevchenko et al., 1996) with modifications. Briefly, gel bands were dehydrated using 100% acetonitrile and incubated with 10 mM dithiothreitol in 100 mM ammonium bicarbonate, pH ˜8, at 56° C. for 45 min, dehydrated again and incubated in the dark with 50 mM iodoacetamide in 100 mM ammonium bicarbonate for 20 min. Gel bands were then washed with ammonium bicarbonate and dehydrated again. Sequencing grade modified trypsin was prepared to 0.005 μg μL⁻¹ in 50 mM ammonium bicarbonate and ˜100 μL of this was added to each gel band so that the gel was completely submerged. Bands were then incubated at 37° C. overnight. Peptides were extracted from the gel by water bath sonication in a solution of 60% Acetonitrile (ACN)/1% Trifluoroacetic acid (TFA) and vacuum dried to ˜2 μL. Digests were re-suspended to 20 μL in 2% ACN/0.1% TFA. An injection of 10 μL was automatically made using a Thermo EASYnLC 1200 onto a Thermo Acclaim PepMap RSLC 0.075 mm×250 mm C18 column with a gradient of 5% B to 40% B in 24 min, ramping to 90% B at 25 min and held at 90% B for the duration of the run (Buffer A=99.9% Water/0.1% Formic Acid, Buffer B=80% Acetonitrile/0.1% Formic Acid/19.9% Water) at a constant flow rate of 300 nL min⁻¹. Column temperature was maintained at a constant temperature of 50° C. using and integrated column oven (PRSO-V2, Sonation GmbH, Biberach, Germany). Eluted peptides were sprayed into a ThermoScientific Q-Exactive HF-X mass spectrometer using a FlexSpray spray ion source. Survey scans were taken in the Orbitrap (60,000 resolution, determined at m/z 200) and the top 15 ions in each survey scan are then subjected to automatic higher energy collision induced dissociation (HCD) with fragment spectra acquired at 15000 resolution. The resulting MS/MS spectra are converted to peak lists using MaxQuant, v1.6.3.4 and searched against a library containing all B. distachyon protein sequences available from Phytozome v13. The MaxQuant output was then analyzed using Scaffold Q+S, v5.1.2 to probabilistically validate protein identifications. Assignments validated using the Scaffold 1% FDR confidence filter are considered true. The mass spectrometry proteomics data have been deposited to the ProteomeXchange Consortium via the PRIDE partner repository (https//www.ebi.ac.uk/pride/) in MIAPE-compliant format with the dataset identifier PXD033835.

### Graphs and Statistical Analyses:

Statistical analyses and plotting were performed with GraphPad Prism 9.3 (GraphPad Software, Inc., San Diego, CA). Data on phenotypic or phytohormone measurements were subjected to statistical analysis by one-way ANOVA or Student's t-test.

### Example 2: Conserved RFS Genes Respond to Multiple Abiotic Stresses

This Example illustrates how conserved RFS genes respond to abiotic stresses.

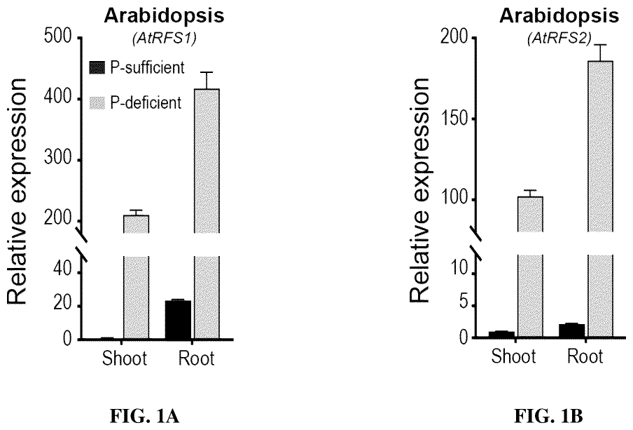

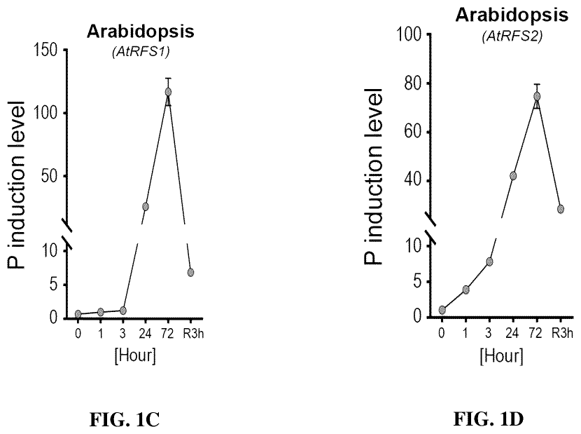

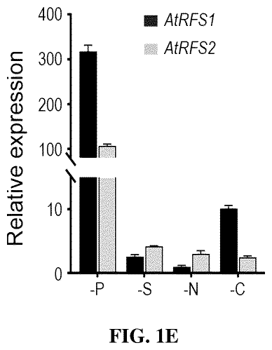

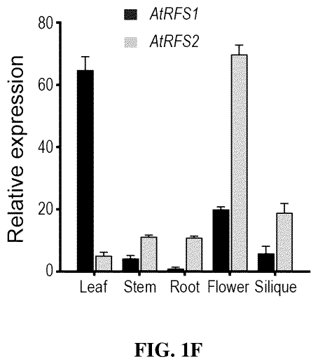

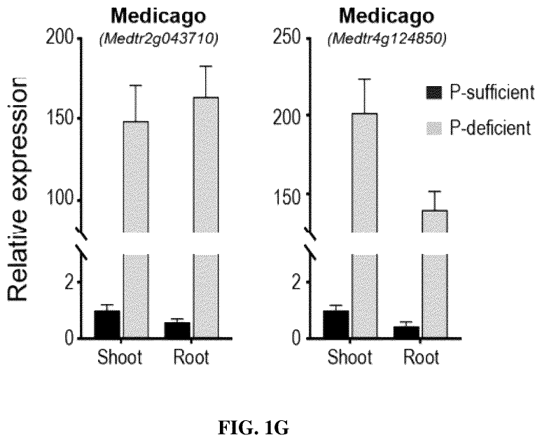

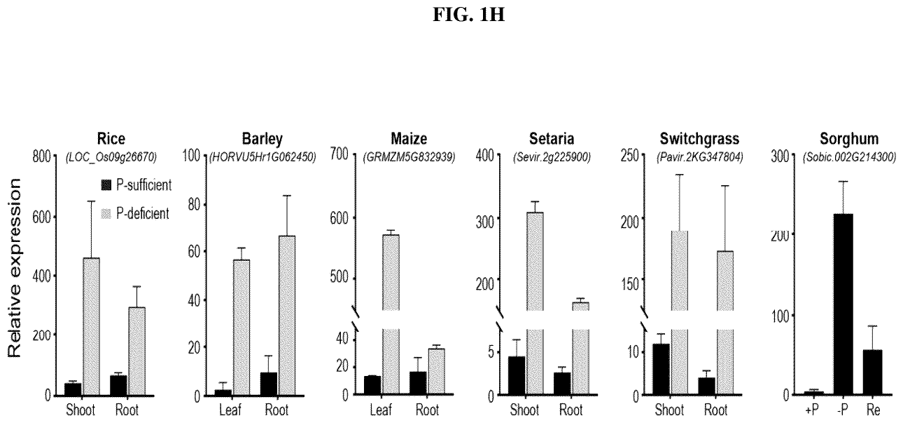

The A. thaliana RFS genes, AtRFS1 (At3g43110) and AtRFS2 (At5g20790), have been proposed as hallmark genes of the P-limitation response (Bari et al., 2006). RT-qPCR assays confirmed the strong induction of both gene transcripts in shoot and root tissue during P-limitation (FIG. 1A-B). Following P re-addition, both transcript levels dropped rapidly within 3 hours (FIG. 1C-D). Notably, AtRFS1 and AtRFS2 gene transcripts were strongly induced by P-limitation only, but either not at all or much less by sulfur, nitrogen and carbon limitation (FIG. 1E). Promoter-GUS staining assays revealed complementary tissue expression patterns between the two homologs. While staining assays revealed no distinguishable GUS signals in P-replete conditions, under P-stress. AtRFS1 was expressed in leaf apex, root meristem and root tip, whereas AtRFS2 was expressed in leaves and primary root, but not in the root tip (images not shown). Furthermore. RT-qPCR assays of P-replete A. thaliana tissue demonstrated that both homologs are expressed at low levels, with AtRFS1 referentially expressed in leaves and AtRFS2 predominant in flowers (FIG. 1F). Furthermore, both homologs are strongly expressed in seeds (images not shown).

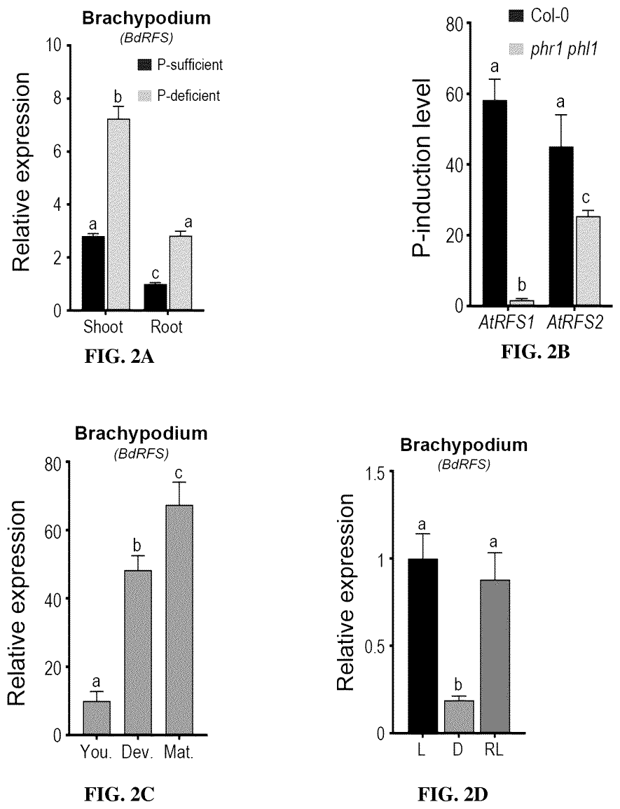

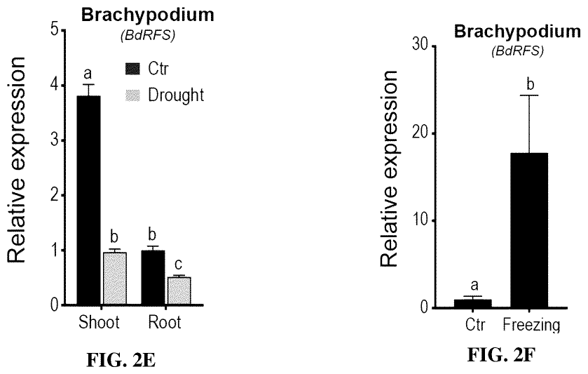

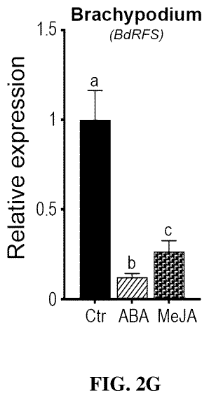

Orthologs of RFS genes were identified in both monocots and eudicots, as shown in SEQ ID NOS: 2, 3, 6, 8, 10, 12, 14, 16, 18, 20, 22, 24, 26, 28, 30, 32, or 34 above. Protein sequence alignment revealed a high degree of conservation in the N-terminal transmembrane domain and C-terminus of proteins (FIG. 12). To better understand their evolutionary relationship, a phylogenetic tree consisting of RFS protein sequences from eight monocot (B. distachyon, Oryza sativa, Zea mays, Sorghum bicolor, Setaria italica, Panicum virgatum, Hordeum vulgare, and Triticum aestivum) and three eudicot species (A. thaliana, Medicago trumcatula, and Glycine max) was constructed. While single copies of RFS were identified in monocot genomes, dicots harbored two homologs (or three in the tetraploid G. max). Monocot and dicot RFS proteins were divided into distinct monocot and dicot clades (data not shown). These results indicate that RFS proteins are conserved throughout the higher plant lineage, and that their sequence conservation is largely concordant with the species tree. Comparable to A. thaliana, the orthologous RFS genes in M. truncatula, O. sativa, and Z. mays were strongly induced by P-limitation in both root and shoot tissue (FIGS. 1G-H). In B. distachyon, RFS genes were also induced although somewhat less than in the other investigated species (FIG. 2A).

PHR1 and PHL1 are transcription factors in P-signaling (Rubio et al., 2001; Nilsson et al., 2007; Bustos et al., 2010). During P-limitation, AtRFS1 and AtRFS2 transcript levels were clearly reduced in a phr1 phl1 double mutant (FIG. 2B), consistent with a previous report (Castrillo et al., 2017), indicating their transcriptional dependence on PHR1 and PHL1 (Bustos et al., 2010). Similarly, abundance of the rice OsRFS gene transcript, LOC_Os09g26670, was dependent on OsPHRs (data not shown). These results suggest that plant RFS genes are integrated within PHR1/PHL1-regulated signaling networks.

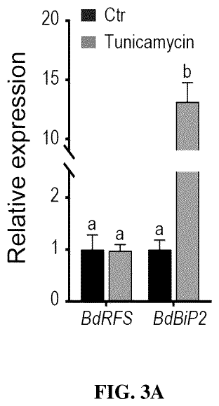

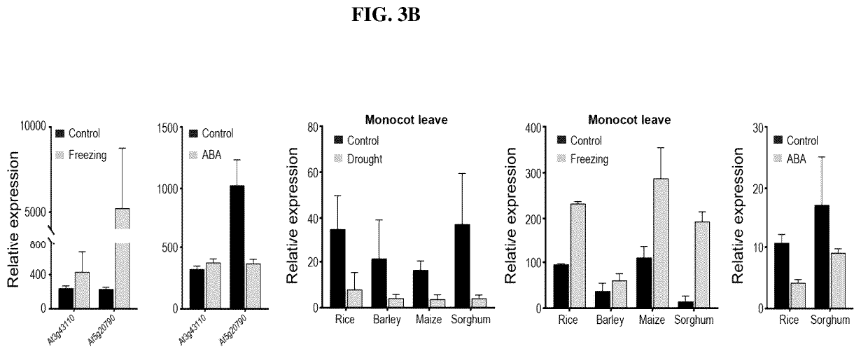

BdRFS transcript increased over 6-fold during leaf maturation, consistent with public datasets for O. sativa and Z. mays (FIG. 2C). On the other hand, BdRFS transcript was reduced in the dark (FIG. 2D). To investigate whether BdRFS transcript abundance may be subject to PHR1 TF, we scanned the promoter region (3 kb upstream of start codon) for PHR1 binding sites (P1BS, GNATATNC, where N represents any base). Two P1BS sequences were found at 231 and 491 bp upstream of the start codon. Moreover, cis-regulatory elements (CREs) related to drought stress, low temperature and ABA/MeJA responses were identified in the BdRFS promoter region. Therefore, we hypothesized that BdRFS might respond to environmental stimuli and endogenous phytohormones. BdRFS transcript abundance was induced during freezing treatment but suppressed during drought or phytohormones (ABA or MeJA) treatments (FIG. 2E-G). P-limitation activates endoplasmic reticulum (ER) stress and then elicits the unfolded protein response (UPR) (Naumann et al., 2019). We treated 7-day-old B. distachyon wild-type (BD21-3) seedlings with tunicamycin (Tm) to stimulate ER stress (Kim et al., 2017). RT-qPCR showed that BdRFS transcript abundance was not induced by Tm treatment (FIG. 3A). RFS genes in other grasses (e.g., rice, barley and maize) or in A. thaliana exhibited similar expression patterns to abiotic stresses (drought and freezing) or ABA treatment (FIG. 3A). Collectively, these findings suggest that plant RFS genes are conserved in higher plants and respond to various environmental stimuli.

### Example 3: RFS Proteins May Localize to Endoplasmic Reticulum and Cytosol

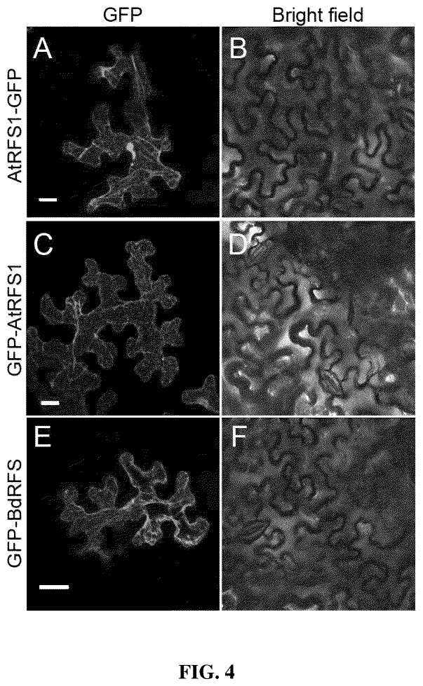

To investigate the subcellular localization of plant RFS proteins, proteomic databases and subcellular localization prediction tools were searched, namely SUBA4, WoLF P-Sort, TargetP, and the Plant Proteome Database (Horton et al., 2007; Sun et al., 2009; Hooper et al., 2016; Almagro Armenteros et al., 2019). Dicotyledonous RFS proteins tended to hold predicted localizations in the cytosol, whereas prediction tools for the monocotyledonous RFSs gave conflicting results (data not shown). Proteomics datasets provided little insight because only a single proteomics study identifying an RFS protein was found in the literature, a study of isolated plasma membrane from A. thaliana cell suspension culture which identified low levels of AtRFS2 (Mitra et al., 2009). Since public databases and prediction tools provided limited and conflicting information on possible subcellular localization, we experimentally tested localization with AtRFS1 tagged N- or C-terminally with GFP (GFP-AtRFS1 and AtRFS1-GFP, respectively), introduced transiently into tobacco leaves. Both fusion orientations demonstrated patterns consistent with localization in the endoplasmic reticulum network and, to a lesser extent, cytosol (FIG. 4). Notably, BdRFS protein was localized in ER and cytosol, consistent with AtRFS1 (FIG. 4).

### Example 4: Overexpression of the BdRFS Gene Delays Flowering and Enhances Drought Tolerance

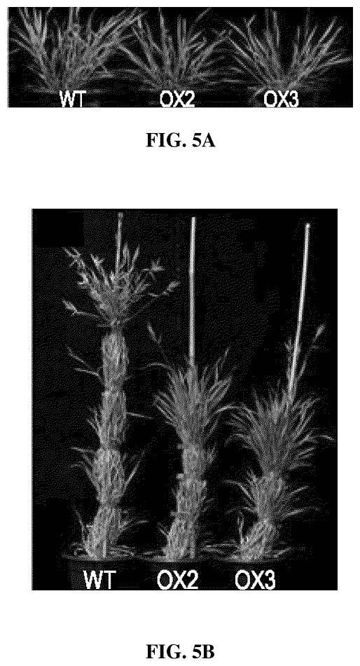

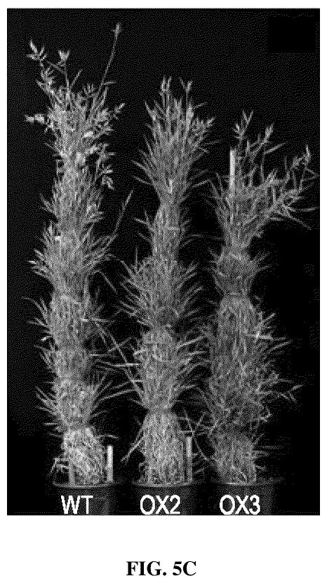

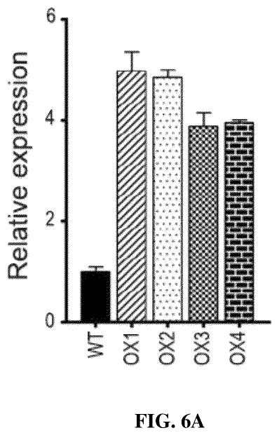

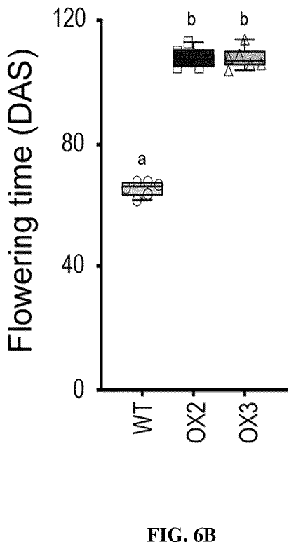

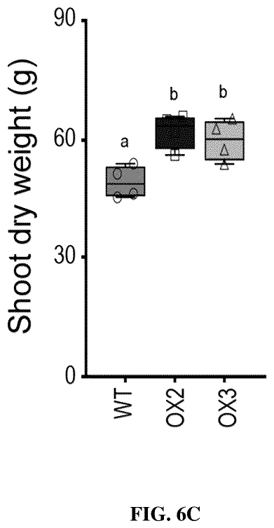

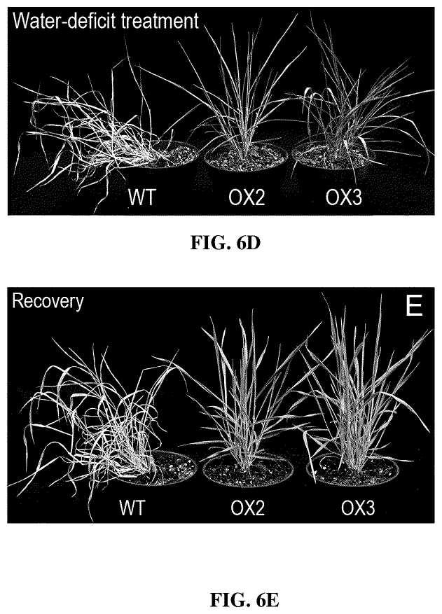

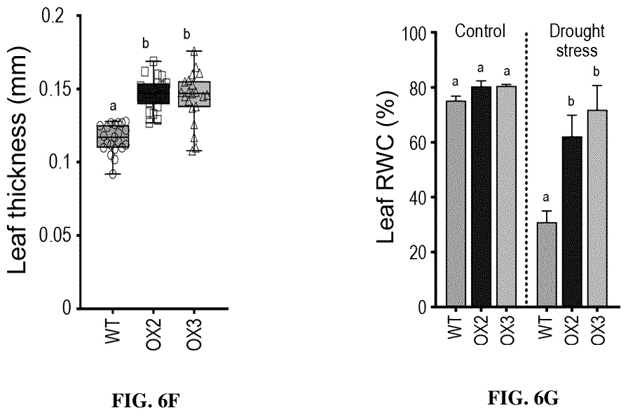

To elucidate the function of BdRFS in B. distachyon, constitutive overexpressor lines were generated. RT-qPCR analysis revealed that BdRFS transcript was significantly (˜4-fold) elevated in four independent transgenic lines (FIG. 6A). Flowering time and senescence were greatly delayed in the BdRFS overexpressor lines (FIGS. 5A and 5C). For example, panicles of wild-type control were visible by about 66 days after sowing (DAS), while those of OX2 appeared after only about 107 DAS. Thus, overexpression of RFS is shown to delay flowering time by approximately forty days as compared to plants not overexpressing RFS. Stated another way, plants overexpressing RFS delay flowering approximately 62% longer than plants not overexpressing RFS. Plants with delayed flowering time produce greater biomass, a desired trait for grazing operations and biofuel feedstocks (Hilbert et al., 1981; Schwartz et al., 2010). Shoot dry weight of overexpressors were significantly increased compared to wild-type (FIG. 6C). Shoot dry weight of the plant overexpressing RFS increased by approximately 20 percent (%) as compared to plants not overexpressing RFS (wild-type). Photosynthetic pigment levels of overexpressors were unchanged, except for a slight but significant increase of plastoquinone-9, which could be in favor of preventing photooxidative damage to thylakoid membranes (data not shown).

Because BdRFS was strongly suppressed by dehydration (FIG. 2E), the drought stress response of BdRFS overexpressing lines was examined. Five-week-old B. distachyon plants in the vegetative growth phase were deprived of water for 2 weeks and then re-watered for 3 days. Wild-type plants exhibited an extremely dehydrated phenotype, marked by dry, necrotic leaf tissue, whereas the leaves of overexpressors remained vigorous throughout the treatment interval (FIG. 6D). After recovery, overexpressors restored growth, but wild-type died (FIG. 6E). Notably, the leaves of overexpressors were significantly thicker than those of wild-type, and had a higher leaf relative water content (RWC) during drought stress (FIGS. 6F and 6G). Leaves of plants overexpressing RFS exhibit approximately 38% to approximately 52% more RWC as compared to plants not overexpressing RFS. Overall, the results suggest that BdRFS not only affects plant flowering time and vegetative biomass production, but also promotes robust tolerance to drought stress.

### Example 5: Knockout Mutants of RFS Exhibit Earlier Flowering and Hypersensitivity to Water Stress

To further investigate its biological functions, BdRFS knockout mutants in B. distachyon were generated using CRISPR/Cas9. Two sgRNA sequences targeting the second and third conserved domain of BdRFS were designed by CRISPR-P (data not shown). Both sgRNAs were simultaneously constructed into the pRGEB32 vector and introduced into wild-type B. distachyon BD21-3 calli via Agrobacterium-mediated transformation. Hygromycin-resistant transformants were selected for further propagation. The effects of BdRFS gene-editing were determined by PCR analysis and confirmed by sequencing. Two independent BdRFS CRISPR-edited knockout mutant lines (CRISPR-ko) were selected for downstream characterization (data not shown). The first line, crispr #3, was the result of a one nucleotide insertion (Adenine, A) at position 366 bp of the coding region, causing an early stop codon at amino acid position 130. The other mutant, crispr #9, contained a 180-nucleotide deletion between the two sgRNAs, resulting in partial deletion of conserved domains 2 and 3.

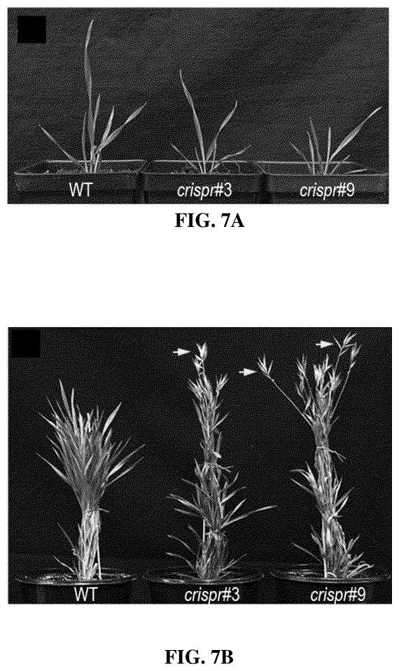

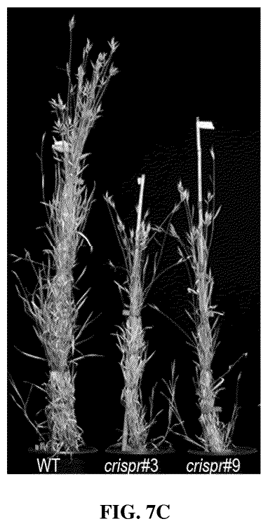

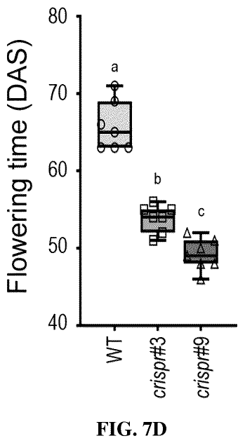

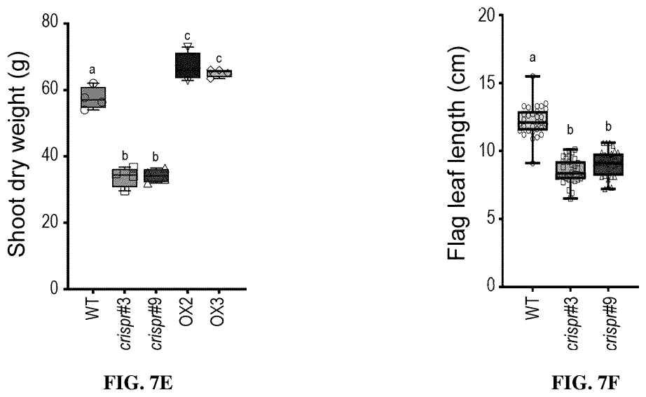

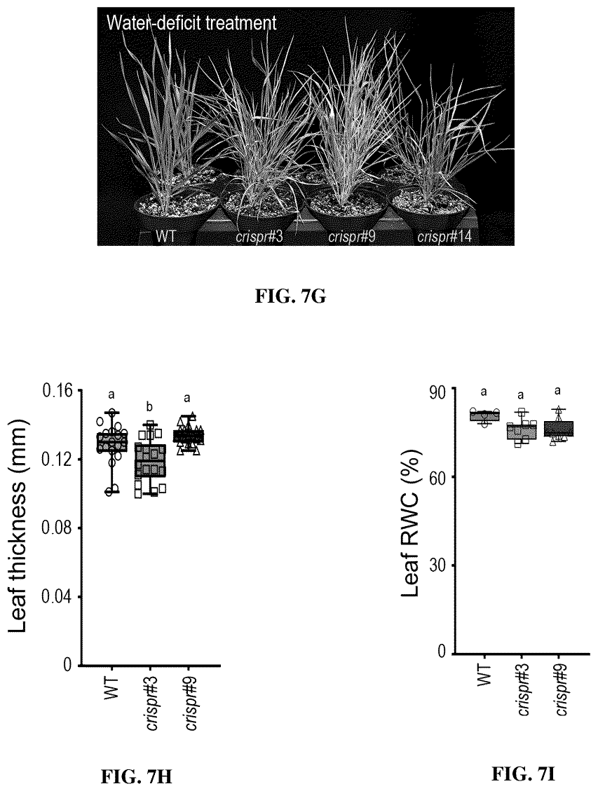

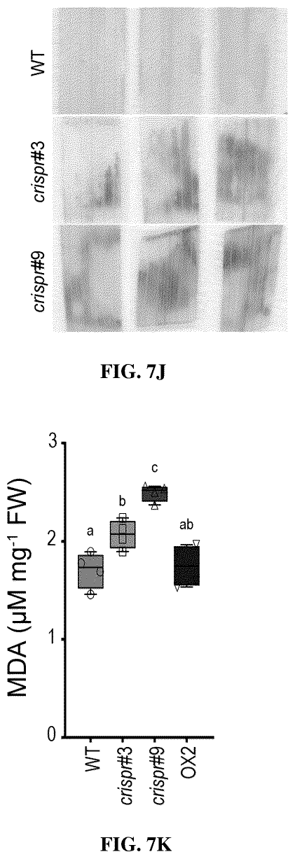

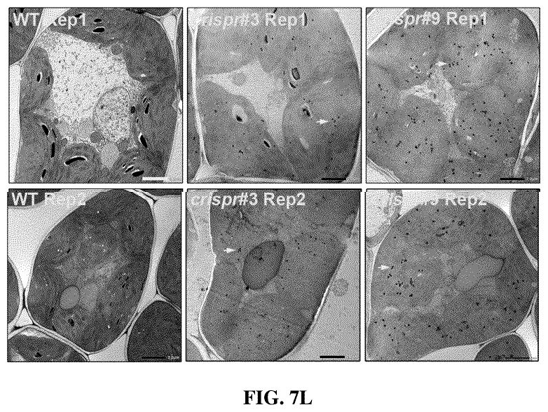

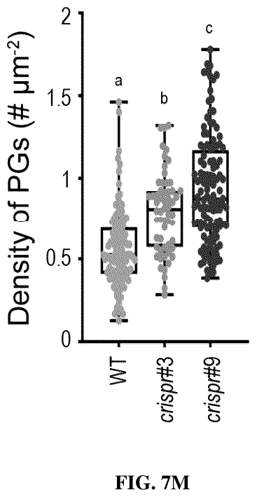

B. distachyon CRISPR-ko mutants displayed a semi-dwarfed phenotype, earlier flowering and reduced biomass accumulation (FIGS. 7A-C). The accelerated flowering phenotype of CRISPR-ko mutants was also observed when grown under an extra-long day (20 hr light and 4 hr dark) photoperiod (data not shown), indicating that the BdRFS gene prolongs flowering time without disturbing photoperiodism. RT-qPCR analysis showed that expression of the Flowering Locus T homologs, BdFT1 (Bd1g48830) and BdFT2 (Bd2g07070), were slightly increased in crispr #9, but not significantly (data not shown). Photosynthetic pigments were reduced in mature leaves of CRISPR-ko mutants (data not shown). However, photosynthetic parameters such as Phi2 (Quantum yield of Photosystem II) and Fv/Fm (the maximum potential quantum efficiency of Photosystem II) were unaffected (data not shown).

Drought stress experiments revealed that the CRISPR-ko mutants were hypersensitive to water stress (FIG. 7G). After withholding water for 7 days, the leaves of knockout mutants already exhibited severe wilting and dehydration symptoms in sharp contrast to wild-type plants. The leaves of crispr #3 were slightly thinner than those of wild-type, but the leaf RWC of the two CRISPR-ko mutants were unchanged (FIG. 7H-1).

The levels of hydrogen peroxide (H2O2) and malondialdehyde (MDA) in leaves of wild-type and CRISPR-ko mutants were also examined. Leaves were sampled at the same developmental stage (i.e., bolting day), to exclude developmental effects. 3,3′-diaminobenzidine (DAB) staining indicated that H2O2 levels were increased in CRISPR-ko mutants under standard growth conditions (FIG. 7. MDA is one of the end products of PUFA peroxidation, and its level reflects oxidative stress status (Draper and Hadley, 1990). The CRISPR-ko mutants accumulated more MDA (FIG. 7K), indicating that either the antioxidant systems were impaired, or reactive oxygen species generation was elevated, in knockout mutants (FIG. 7GK). Thylakoid-associated plastoglobules (PGs) are monolayer lipid droplet particles of chloroplasts. The size and density of PGs can vary substantially under adverse conditions (Wijk and Kessler, 2017). Transmission electron microscopy revealed that the density, but not the size, of PGs was significantly increased in CRISPR-ko mutants (FIG. 7L-M). Together, the results suggest that BdRFS is a stress-inducible regulator of plant flowering time and drought tolerance.

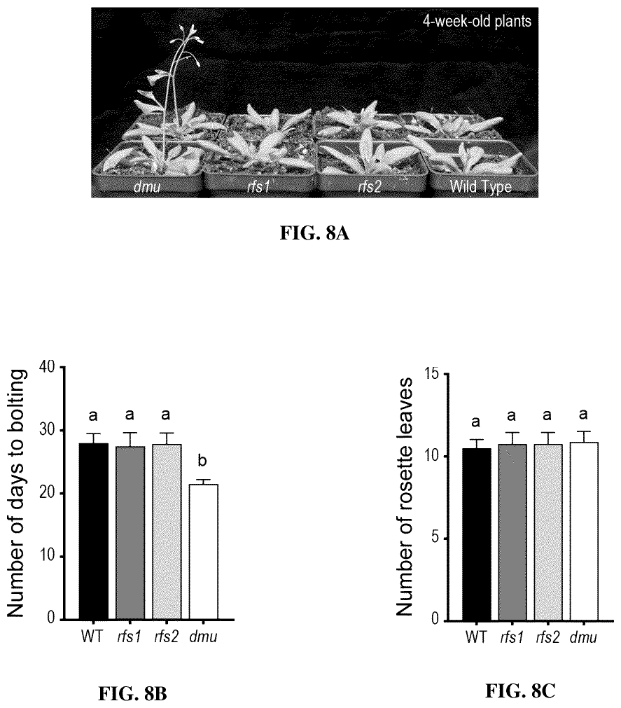

To investigate conservation of function among the RFS family, T-DNA single mutants of the two A. thaliana homologs, At3g43110 (atrfs1) and At5g20790 (atrfs2), were isolated and a corresponding double homozygous mutant (dmu) was generated. Morphological differences were not observed from seedlings of single and double mutants under P-replete or -deficient conditions (data not shown). However, it was observed that dmu bolted earlier than wild type or single mutants (FIGS. 8A and 8B). The number of rosette leaves at bolting, and mature plant height, did not show any significant differences (FIG. 8C). Notably, dmu exhibited water deficit sensitivity (data not shown), which is consistent with the observation in the B. distachyon CRISPR-ko lines. Thus, the A. thaliana RFS genes synergistically affect transition to reproductive growth and drought tolerance, comparable to the role of the B. distachyon homolog.

### Example 6: RFS Genes Regulate Lipid Composition and Lipid Acyl-Group Composition

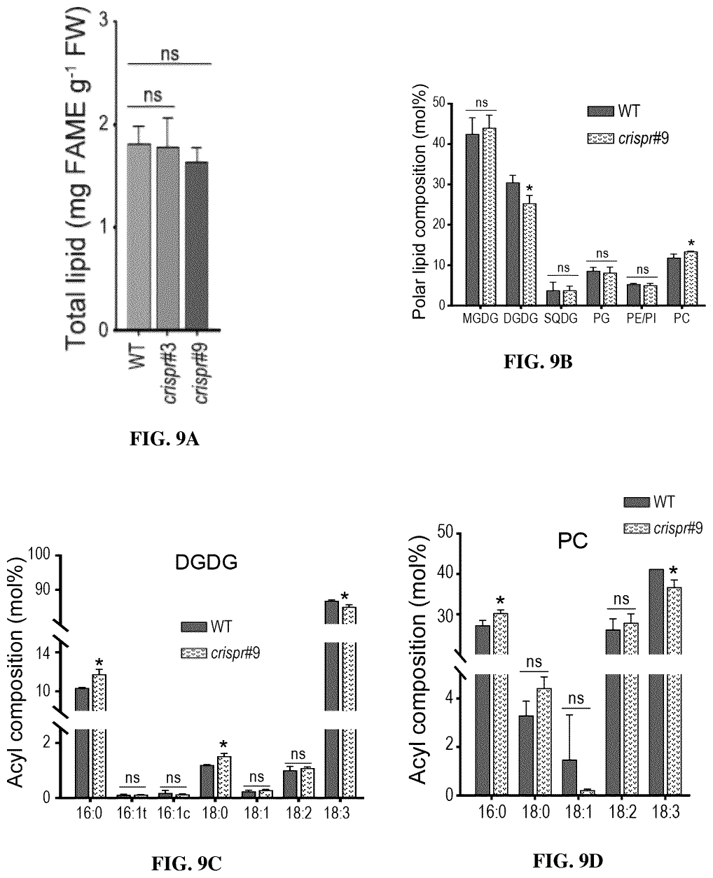

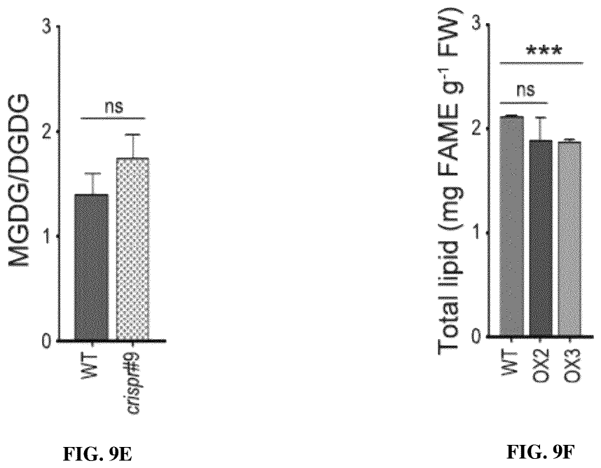

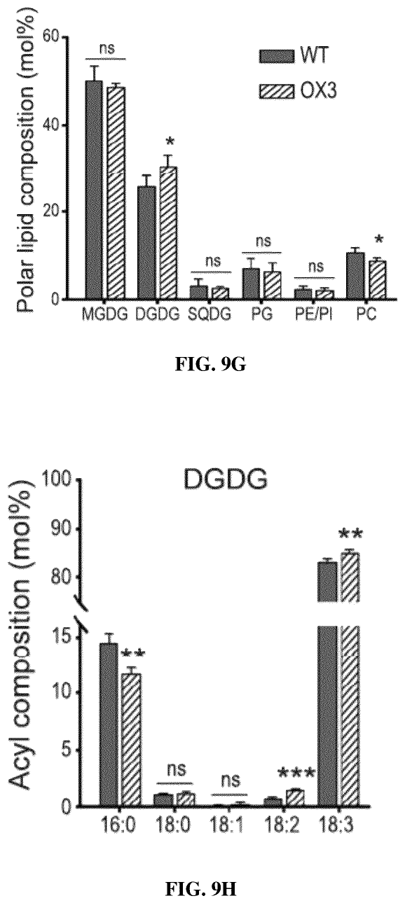

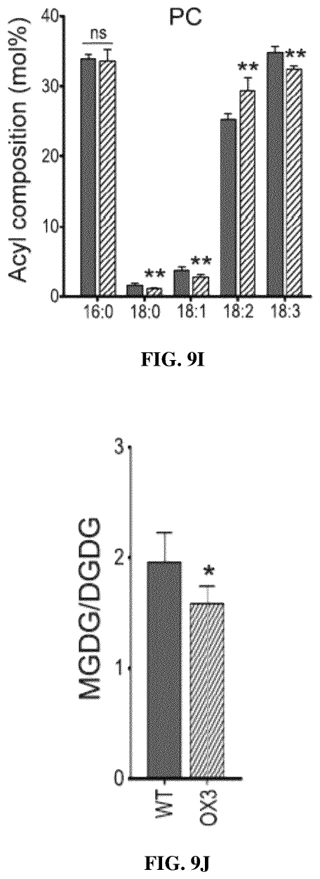

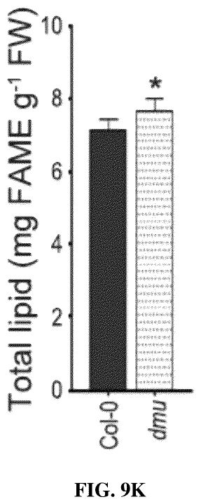

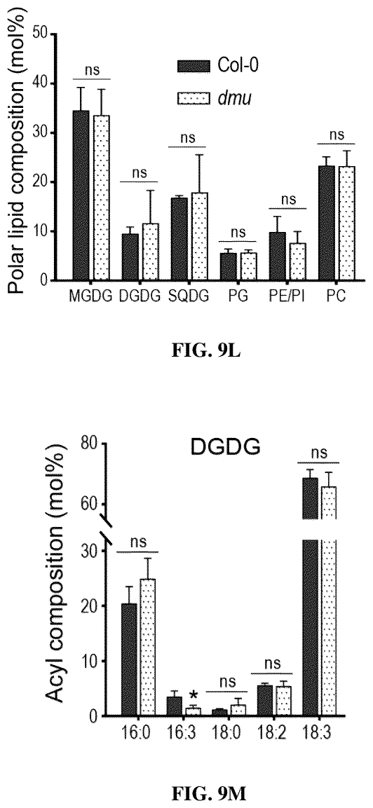

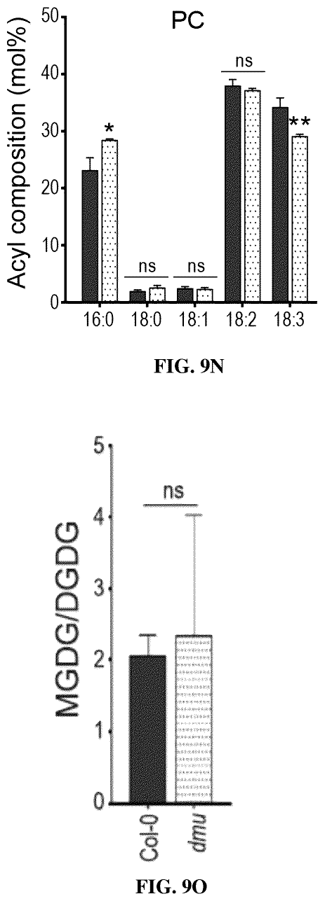

Previous studies have shown that changes in phosphatidylcholine (PC) content in total lipids affect plant flowering/heading time (Nakamura et al., 2014; Qu et al., 2021). Furthermore, the accumulation of malondialdehyde (MDA) in CRISPR-ko mutants suggested that unsaturated membrane lipids might be subjected to peroxidation and destabilization. To investigate if changes to lipid composition may underlie the developmental phenotype, the lipid profiles of the BdRFS CRISPR-ko mutant lines and overexpressors were investigated. Although the total free fatty acid (FA) content of CRISPR-ko mutants did not change compared to wild-type (FIG. 9A), in crispr #9, the DGDG content was significantly reduced, while the PC content was increased (FIG. 9B). The distribution of acyl groups of free FAs was altered in each lipid class, with a slight but significant decrease in C18:3 and an increase in C16:0, as shown in Table 2 below.

TABLE 2
Acyl group distribution (mol %) of each lipid species in BD21-3 and crispr#9 mutant

| | | | C16:1 | | | | | |
|---|---|---|---|---|---|---|---|---|
| | | C16:0 | Cis | C16:1 Trans | C18:0 | C18:1 | C18:2 | C18:3 |
| BD21-3 | MGDG | 1.04 ± 0.04 | 0.09 ± 0.01 | 0.13 ± 0.01 | 0.14 ± 0.01 | 0.14 ± 0.01 | 1.30 ± 0.15 | 97.15 ± 0.20 |
| | DGDG | 10.37 ± 0.06 | 0.12 ± 0.01 | 0.19 ± 0.09 | 1.20 ± 0.01 | 0.25 ± 0.04 | 1.01 ± 0.14 | 86.87 ± 0.22 |
| | SQDG | 27.36 ± 3.81 | ND | ND | 1.83 ± 0.24 | ND | 3.62 ± 0.03 | 66.97 ± 3.76 |
| | PG | 16.23 ± 1.43 | ND | 39.85 ± 0.12 | 0.93 ± 0.10 | 0.20 ± 0.03 | 3.38 ± 0.33 | 39.41 ± 1.99 |
| | PC | 27.35 ± 1.19 | ND | ND | 3.31 ± 0.59 | 1.49 ± 1.84 | 26.29 ± 2.61 | 41.28 ± 0.01 |
| | PE/PI | 24.43 ± 1.26 | ND | ND | 1.05 ± 0.06 | 0.93 ± 0.02 | 34.20 ± 1.52 | 39.39 ± 0.18 |
| crispr#9 | MGDG | 1.34 ± 0.11* | 0.12 ± 0.01 | 0.20 ± 0.02** | 0.19 ± 0.02* | 0.17 ± 0.02 | 1.63 ± 0.17 | 96.34 ± 0.29* |
| | DGDG | 11.76 ± 0.48* | 0.13 ± 0.01 | 0.14 ± 0.01 | 1.53 ± 0.09* | 0.29 ± 0.02 | 1.09 ± 0.03 | 85.06 ± 0.62* |
| | SQDG | 33.31 ± 1.81 | ND | ND | 2.27 ± 0.37 | ND | 4.36 ± 0.67 | 59.94 ± 1.89 |
| | PG | 23.31 ± 0.63** | ND | 36.61 ± 1.01* | 0.96 ± 0.04 | 0.27 ± 0.03 | 4.14 ± 0.39 | 34.71 ± 0.88* |
| | PC | 30.37 ± 0.73* | ND | ND | 4.43 ± 0.47 | 0.23 ± 0.03 | 27.98 ± 2.13 | 36.80 ± 1.69* |
| | PE/PI | 26.27 ± 1.37 | ND | ND | 1.46 ± 0.31 | 0.94 ± 0.23 | 40.72 ± 1.89* | 30.61 ± 0.48*** |

The data represent the molar ratio of total lipids (mol %) and represent the mean values of three replicates ± SD. Acyl groups are designated with numbers of carbons:number of double bonds. *P < 0.05, **P < 0.01, ***P < 0.001, and ****P < 0.0001 indicate statistical significance as determined by Student's t test. ND, not detected.

For example, the C16:0 level in PC of crispr #9 was approximately 10% more than that of wild-type (FIG. 9D). Moreover. C18:0 level in DGDG was also increased in crispr #9 (FIG. 9C). The double bond index (DBI) reflects the degree of unsaturation of membrane lipids. The DBI levels of CRISPR-ko mutants were significantly lower than wild-type (data not shown). In contrast, OX3 had significantly lower total FA content (FIG. 9F). The DGDG content of OX3 was increased, while its PC content was decreased, the reverse of the pattern observed in the CRISPR-ko mutants (FIG. 9G). Reciprocal alteration of PC and DGDG levels is repeatedly Observed in P-starved plants (Pfaff et al., 2020; Sun et al., 2020). Alterations to DGDG content consequently affected the MGDG/DGDG ratio (FIGS. 9E and 9J). Levels of C18:2 in total FAs was significantly increased in overexpressor lines, as shown in Table 3 below.

TABLE 3
Acyl groups distribution (mol %) of each polar lipid species in BD21-3 and overexpression line #3.

| | Polar | | C16:1 | | | | | |
|---|---|---|---|---|---|---|---|---|
| Genotype | Lipid | C16:0 | Cis | C16:1 Trans | C18:0 | C18:1 | C18:2 | C18:3 |
| BD21-3 | MGDG | 1.43 ± 0.08 | ND | ND | 0.13 ± 0.09 | 0.17 ± 0.13 | 1.44 ± 0.11 | 96.83 ± 0.15 |
| | DGDG | 14.56 ± 0.80 | ND | ND | 1.20 ± 0.04 | ND | 0.83 ± 0.10 | 83.26 ± 0.63 |
| | SQDG | 39.05 ± 3.97 | ND | ND | 2.45 ± 0.50 | ND | 5.09 ± 0.62 | 52.95 ± 3.64 |
| | PG | 16.58 ± 1.24 | ND | 49.11 ± 1.25 | 1.51 ± 0.51 | ND | 2.45 ± 0.28 | 30.12 ± 1.83 |
| | PC | 34.05 ± 0.53 | ND | ND | 1.82 ± 0.19 | 3.87 ± 0.42 | 25.34 ± 0.69 | 34.91 ± 0.76 |
| | PE/PI | 34.06 ± 0.86 | ND | ND | ND | ND | 33.87 ± 1.65 | 30.50 ± 1.03 |
| OX3 | MGDG | 1.26 ± 0.14* | ND | ND | 0.19 ± 0.03 | 0.26 ± 0.10 | 2.60 ± 0.41** | 95.68 ± 0.56** |
| | DGDG | 11.74 ± 0.53** | ND | ND | 1.27 ± 0.11 | ND | 1.54 ± 0.07**** | 85.04 ± 0.63** |
| | SQDG | 39.96 ± 4.50 | ND | ND | 2.54 ± 1.94 | ND | ND | 55.65 ± 1.67 |
| | PG | 15.94 ± 0 30** | ND | 49.47 ± 1.09 | 1.13 ± 0.12 | 1.48 ± 0.39 | 4.37 ± 0.30**** | 27.61 ± 0.26* |
| | PC | 33.74 ± 1.50 | ND | ND | 1.37 ± 0.03** | 2.96 ± 0.26** | 29.48 ± 1.69** | 32.45 ± 0.37** |
| | PE/PI | 34.67 ± 3.02 | ND | ND | 2.17 ± 1.56 | ND | 40.46 ± 1.70** | 22.69 ± 1.90*** |

The data represent the molar ratio of total lipids (mol %) and represent the mean values of three replicates ± SD. Acyl groups are designated with numbers of carbons:number of double bonds. *P < 0.05, **P < 0.01, ***P < 0.001, and ****P < 0.0001 indicate statistical significance as determined by Student's t test. ND, not detected.

Additionally, in OX3, the level of C16:0 in DGDG was substantially decreased, whereas the C18:2 and C18:3 levels were significantly increased (FIG. 9H). The level of C18:2 in PC was also increased, but C18:0, C18:1, and C18:3 levels were noticeably decreased (FIG. 9I). Notably, the double bond index (DBI) levels of overexpressors were comparable to wild-type (data not shown).

The lipid profiles of the atrfs double mutant (dmu), which exhibited the earlier bolting (transition to reproductive growth) phenotype, was investigated (FIG. 8A). Compared to wild-type (Col-0), dmu contained a higher total lipid content, but its relative proportions of lipid classes were unchanged (FIGS. 9K and 9L). The abundance of C16:3 in DGDG was decreased in dmu (FIG. 9M), as also shown in Table 4 below.

TABLE 4
Acyl group distribution (mol %) of each polar lipid
species in Col-0 and Arabidopsis dmu mutant.

| | Polar | | C16:1 | | | |
|---|---|---|---|---|---|---|
| Genotype | Lipid | C16:0 | Cis | C16:1 Trans | C16:2 | C16:3 |
| Col-0 | MGDG | 3.69 ± 0.65 | ND | ND | 1.41 ± 0.07 | 30.73 ± 0.35 |
| | DGDG | 20.45 ± 3.06 | ND | ND | ND | 3.57 ± 0.99 |
| | SQDG | 54.46 ± 7.89 | ND | ND | ND | ND |
| | PG | 24.08 ± 3.15 | ND | 39.73 ± 1.78 | ND | ND |
| | PC | 23.18 ± 2.16 | ND | ND | ND | ND |
| | PE/PI | 32.76 ± 2.99 | ND | ND | ND | ND |
| dmu | MGDG | 2.76 ± 0.65 | ND | ND | 1.37 ± 0.19 | 29.79 ± 1.52 |
| | DGDG | 24.93 ± 3.70 | ND | ND | ND | 1.54 ± 0.40 |
| | SQDG | 60.07 ± 2.09 | ND | ND | ND | ND |
| | PG | 25.79 ± 2.04 | ND | 45.59 ± 1.67* | ND | ND |
| | PC | 28.51 ± 0.13* | ND | ND | ND | ND |
| | PE/PI | 43.48 ± 1.47** | ND | ND | ND | ND |

| | Polar | | | | |
|---|---|---|---|---|---|
| Genotype | Lipid | C18:0 | C18:1 | C18:2 | C18:3 |
| Col-0 | MGDG | ND | ND | 3.69 ± 0.31 | 59.56 ± 1.44 |
| | DGDG | 1.21 ± 0.14 | ND | 5.62 ± 0.34 | 68.78 ± 2.67 |
| | SQDG | 3.94 ± 0.98 | ND | 17.67 ± 2.68 | 23.93 ± 6.34 |
| | PG | ND | 3.28 ± 0.23 | 8.80 ± 0.36 | 24.12 ± 4.64 |
| | PC | 2.00 ± 0.17 | 2.52 ± 0.27 | 38.04 ± 1.03 | 34.26 ± 1.58 |
| | PE/PI | ND | ND | 42.16 ± 1.10 | 23.23 ± 1.36 |
| dmu | MGDG | ND | ND | 2.97 ± 0.30 | 63.11 ± 1.41* |
| | DGDG | 2.13 ± 1.04 | ND | 5.46 ± 0.86 | 65.84 ± 4.66 |
| | SQDG | 4.28 ± 0.42 | ND | 14.56 ± 0.51 | 21.09 ± 1.67 |
| | PG | 2.81 ± 0.55 | 2.66 ± 0.54 | 6.69 ± 0.56** | 16.45 ± 1.63 |
| | PC | 2.65 ± 0.34 | 2.40 ± 0.19 | 37.24 ± 0.26 | 29.19 ± 0.26** |
| | PE/PI | ND | ND | 37.05 ± 1.31** | 16.95 ± 1.48** |

The data represent the molar ratio of total lipids (mol %) and represent the mean values of three replicates ± SD. Acyl groups are designated with numbers of carbons:number of double bonds. *P < 0.05, **P < 0.01, ***P < 0.001, and ****P < 0.0001 indicate statistical significance as determined by Student's t test. ND, not detected.

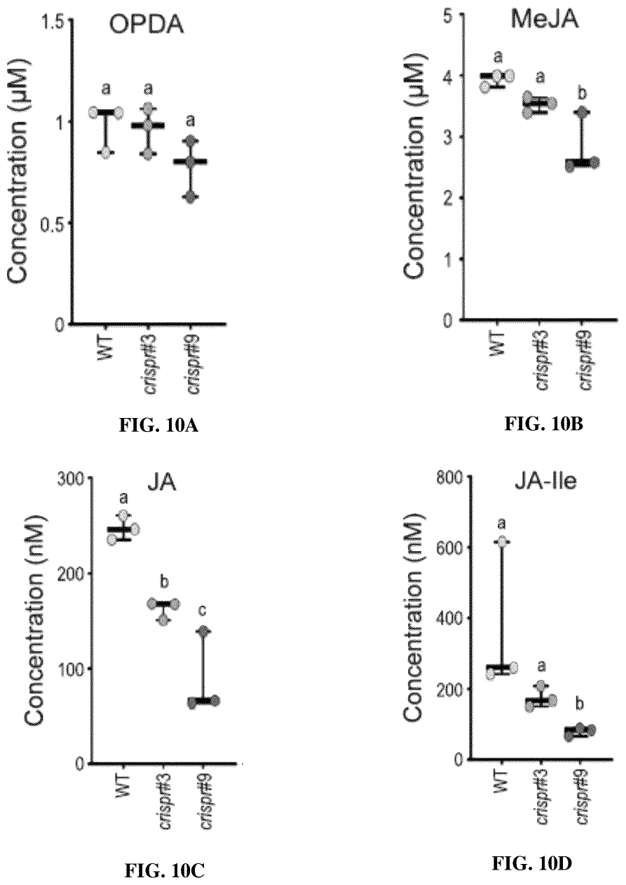

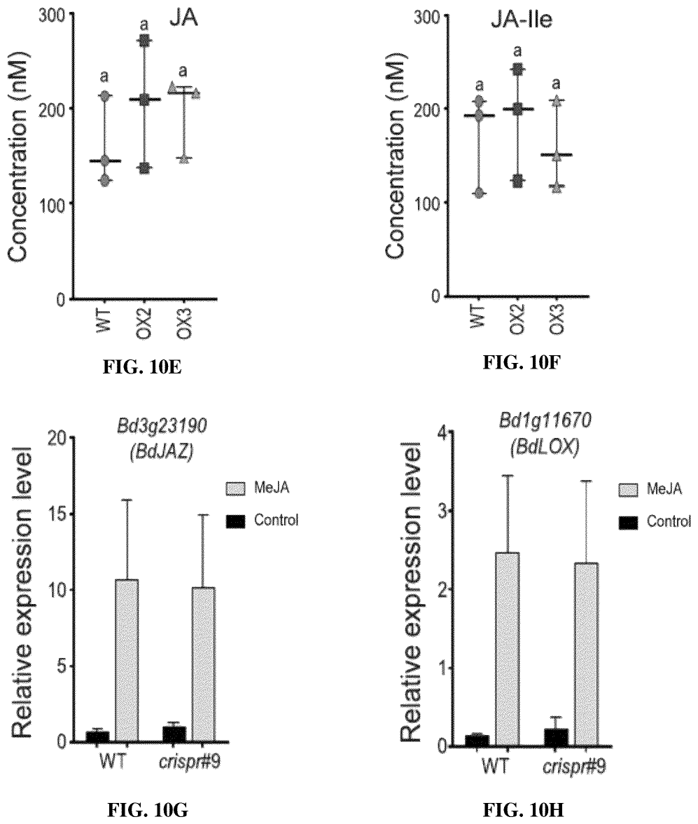

Similar to B. distachyon crispr #9, levels of C16:0 and C18:3 in PC were respectively increased and decreased in dmu (FIG. 9N). The MGDG/DGDG ratio of dmu, however, did not change (FIG. 9N). Collectively, the results suggest that plant RFS genes affect polar lipids, particularly the acyl chain composition, in a specific and conserved manner. Endogenous jasmonic acid abundance is reduced in BdRFS CRISPR-ko mutants. The PUFA, α-linolenic acid (C18:3), is the precursor of jasmonic acid (JA) biosynthesis (Wasternack and Song, 2016). Because the level of C18:3 FA was decreased in CRISPR-ko mutants, we hypothesized that the JA and related metabolites might be reduced as well. To test this hypothesis, we examined endogenous phytohormones from different genotypes. Despite no change in the 12-oxophytodienoic acid (OPDA, intermediate of JA biosynthesis) level (FIG. 10A), levels of JA, methyl JA (MeJA) and JA-Isoleucine (JA-Ile) were reduced in crispr #9 (FIGS. 10A-B). However, the JA and JA-Ile levels were not altered in overexpressors (FIGS. 10E and 10F). Next, four B. distachyon JA-inducible genes were selected. BdJAZ (Bd3g23190), BdLOX (Bd1g11670). BdMYC2 (Bd3g34200), and BdOPR3 (Bd3g37650) based on previous studies (Kakei et al., 2015; Kouzai et al., 2016) and examined their transcriptional changes after MeJA treatment, to determine whether JA signaling in crispr #9 was altered. Similar to the responses in wild type, all tested genes were up-regulated after MeJA treatment (FIGS. 10G to 10I), indicating that JA signaling was unaffected in crispr #9. The results suggest that BdRFS affects JA biosynthesis but not signal transduction.

### Example 7: Identification of Candidate Interacting Proteins of BdRFS

To reveal the molecular mechanisms underlying the role of BdRFS, co-immunoprecipitations were performed to identify putative protein interactors. Due to high insolubility of the full-length heterogeneously expressed BdRFS protein, even with an N-terminal SUMO tag, the first 45 amino acids of BdRFS were deleted, encompassing its predicted transmembrane domain. Solubility was greatly improved in this truncated form (His-SUMO-BdRFS^{Δ1-45}) and hence was used for the subsequent co-immunoprecipitation assay alongside an empty His-SUMO tag as negative control. Proteins were incubated with crude protein extracts from mature wild-type leaf tissue and eluates were analyzed by LC-MS/MS. A total of 46 potential interactors were identified specifically in the presence of BdRFS^{Δ1-45}, as shown in Table 5 below.

Remarkably, the highest abundant candidate interactor was Abscisic acid-, Stress- and Ripening-induced 2 (BdASR2, Bd4g24650), homologs of which are positive regulators of drought stress tolerance with very similar overexpression phenotypes to the BdRFS overexpressor lines (Wang et al., 2016; Yoon et al., 2019; Qiu et al., 2021; Yoon et al., 2021). Also of particular interest, Phospholipase D al (BdPLD α1, Bd2g04480) was identified as a putative BdRFS-interacting protein. The A. thaliana PLD al preferentially hydrolyzes PC to phosphatidic acid (PA), and the latter acts as an essential lipid signaling molecular in regulation of freezing or drought stress and ABA responses (Wang, 2005; Wang et al., 2014; Hong et al., 2016; Takáč et al., 2019). Furthermore, overexpression of AtPLD α1 significantly enhances drought tolerance by regulating ABA-dependent stomatal movements and transpirational water loss (Sang et al., 2001; Hong et al., 2008). Notably, six different putative interacting proteins are associated with abscisic acid signaling or biosynthesis, including the two aforementioned proteins, BdASR2, and BdPLD α1 (Table 5).

TABLE 5
Selected Candidate Interacting Proteins of BdRFS Determined by Co-Immunoprecipitation

| Protein ID | Annotation | Eluate (BdRFS) ^{a} | Eluate (Neg Ctrl) ^{a} | Input ^{a} | Notes |
|---|---|---|---|---|---|
| Bradi4g24650 | ASR2 (Abscisic acid-, stress- and | 51,856,000 | — ^{b} | — ^{b} | Responsive to ABA and various abiotic |
| | ripening-induced 2) | | | | stresses; overexpression of a wheat |
| | | | | | homolog in B distachyon strongly |
| | | | | | promotes drought stress tolerance and |
| | | | | | ABA accumulation |
| Bradi2g01850 | LPR2 (Low Phosphate Root 2; | 25,251,000 | — ^{b} | — ^{b} | Responsive to P-starvation; adjusts root |
| | cupredoxin family) | | | | meristem activity; copper oxidase domain |
| Bradi5g12120 | PLAT1 (Polycystin, Lipoxygenase, | 22,478,000 | — ^{b} | — ^{b} | Lipase/lipoxygenase-like protein; |
| | Alpha-toxin, and Triacylglycerol | | | | responsive to various (a)biotic stresses |
| | lipase 1) | | | | |
| Bradi4g00330 | NCED1 (Nine-cis Epoxycarotenoid | 14,836,200 | — ^{b} | — ^{b} | Committed enzymatic step of ABA |
| | Cleavage Dioxygenase 1) | | | | biosynthesis |
| Bradi2g04480 | PLD-α1 (Phospholipase D alpha 1) | 9,165,100 | — ^{b} | 6,092,600 | Responsive to ABA signaling, and positive |
| | | | | | regulator of ABA signaling; cleaves |
| | | | | | terminal phosphodiester bond of |
| | | | | | phospholipids to generate phosphatidic |
| | | | | | acid |

^{a} average LFQ intensity from two bioreplicates
^{b} not detected

### Example 8: Ectopic Expression of BdRFS Significantly Affects Stomatal Density and Size

Materials and Methods for Examples 8 and 9:

The Brachypodium transgenic material and Arabidopsis mutant (dmu) used in this study were generated previously (Ying et al., 2023). Brachypodium seeds (BD21-3 and transgenic lines) were soaked in double-distilled water (ddH2O) and kept for 3 days in the dark at 4° C. Then, stratified seeds were planted into soil (SureMix) and grown in a greenhouse with the following conditions: natural light plus high-pressure sodium grow light, 24° C., and a 20/4-h light/dark cycle. For drought stress, three-week-old plants were deprived of water for 7 days. For ABA treatment, 100 μM of ABA was sprayed on the leaves of three-week-old plants and covered with plastic wrap for 30 min. Target tissues were sampled at indicated time points, quickly frozen in liquid N₂ and stored at −80° C. for further studies.

For the water deprivation treatment, four-week-old chamber-grown plants were deprived of water for 10 days. Photographs were captured on day 10, after which aboveground tissue from each plant was collected, weighed, and dried at 65° C. for 48 h. Water content (%) was calculated as (fresh weight-dry weight)×100/fresh weight. Experiments were repeated for three times, and each biological replicate had four to eight plants of each genotype. All experiments were repeated for three times, and representative results were shown in figures.

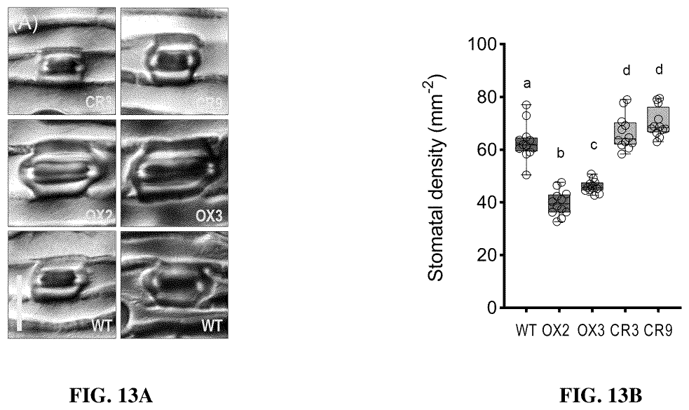

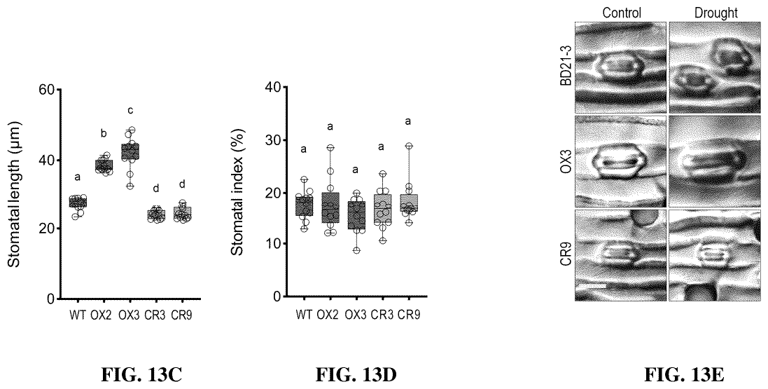

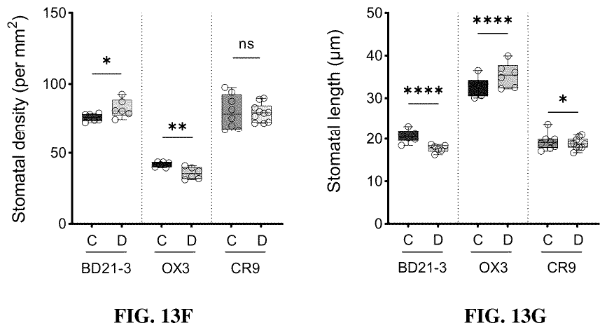

Overexpression of BdRFS in Brachypodium enhanced drought tolerance by reducing leaf water loss. Stomatal anatomy was therefore investigated on the abaxial and adaxial surfaces of mature leaves in overexpression lines (OXs) (FIGS. 13A-D). The morphology of the stomatal complex, formed by four cells, was unchanged in OXs (FIG. 13A), however, stomatal density (SD) on the abaxial surface of OX leaves (40 to 46 stomata mm-2) was reduced by 27%-37% compared to that of BD21-3 (WT) leaves (63 stomata mm-2, FIG. 13B). In contrast, the stomatal length (SL) of OXs (37 to 38 μm on average) was 43%-50% longer than that of WT leaves (26 μm on average, FIG. 13C). On the other hand, the BdRFS CRISPR/Cas9-mediated loss-of-function mutants (i.e., CR3 and 9) exhibited opposite stomatal changes of OXs. For instance, compared to WT, the SD was increased by 12% and SL decreased by 10% in CR9 (FIGS. 13A-C). The stomatal index (SI) did not significantly change in any of the genotypes (FIG. 13D). Similar trends of stomatal change were observed from the adaxial side of the leaf blades of the genotypes (data not shown).

After a 10-day water deprivation treatment, the SD and SL of WT were 14% increased, respectively decrease by 15% (FIGS. 13E-G), compared to well-watered (control) conditions. In contrast, the SD was decreased by 15% and SL increased by 8% in stressed OX3, relative to control conditions. Drought stress only negatively affected the SL of CR9 (8% decrease) but not the SD.

Time-course measurements of water loss using excised mature leaves (after heading) of different genotypes confirmed that the transcript level of the BdRFS gene negatively correlated to the rate of water loss. Because the genotypes exhibit divergent heading times and leaf lengths, the comparisons of water loss rate are performed after OXs or CRs headed, which led to the significantly different measurements of WT between the two sets of experiments. Collectively, the results suggest that BdRFS modulates SD and SS to control leaf water loss.

### Example 9: Ectopic Expression of BdRFS Significantly Affects Biomass and Yield

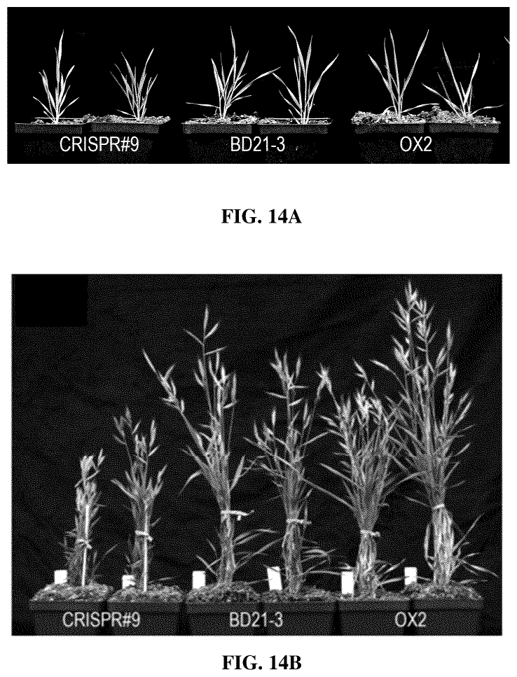

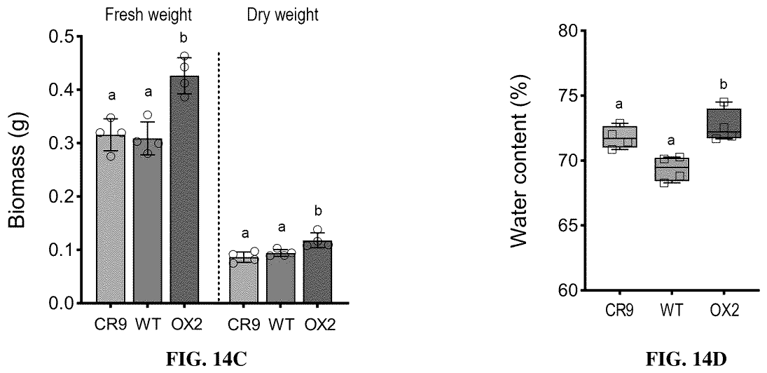

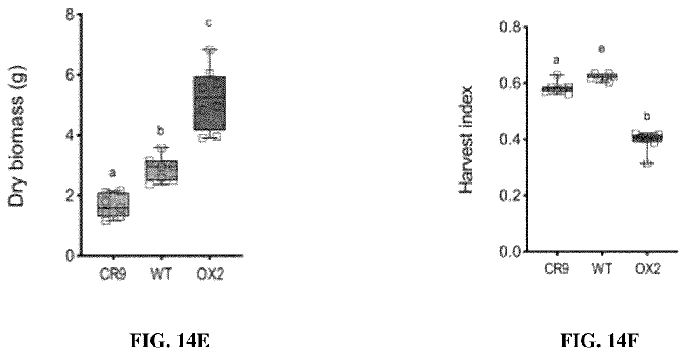

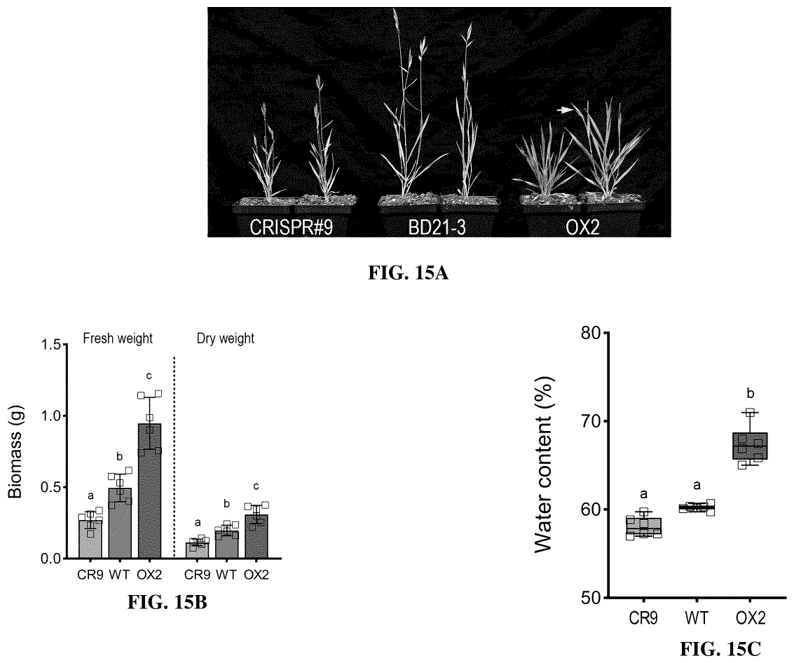

Whether the BdRFS-driven stomatal changes affect plant agronomic traits (FIGS. 14 and 15) were investigated. Under well-watered conditions, the fresh and dry biomass and water content of 4-week-old OX2 plants were 38%, 25%, and 5% higher than those of WT, respectively (FIGS. 14A, C, D). Note that CR9 was already heading at the time of sampling. After plant senescence, the dry weight of OX2 was 82% higher and 43% lower in CR9 than in WT (FIGS. 14B and 14E). Total grain dry weight of each plant and from each genotype was collected to calculate the harvest index (HI, the ratio of grain to total aboveground tissue dry matter). The HI of OX2 was reduced by 37% compared to WT, whereas CR9 exhibited a HI comparable to WT (FIG. 14F).

The water deprivation treatment significantly advanced the heading time of each genotype. For OX2 in particular, by day 10, 33% (2/6) of the drought-stressed plants headed, while none of the well-watered plants did (FIG. 15A). Under drought stress, the fresh and dry weights of OX2 were respectively 89% and 57% higher than that of WT. In contrast, the fresh and dry weights of CR9 decreased by 46% and 43%, respectively (FIG. 15B). Furthermore, the water content of OX2 was 12% higher than that of WT and CR9 (FIG. 15C). Taken together, OX2 and CR9 seemingly adopted different strategies to cope with the water deficit. The drought-avoiding OX2 was prompted to reduce water loss, whereas the drought-escaping CR9 accelerated heading time.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following statements describe some of the elements or features of the invention. The statements provide features that can be claimed in the application and the dependencies of the statements illustrate combinations of features that can be present when included in the claims.

Statements:
1. A plant cell, plant seed, or plant comprising at least one heterologous promotor operably linked to a nucleic acid segment encoding a RFS polypeptide that has at least 95% sequence identity to any of SEQ ID NOS: 2, 3, 6, 8, 10, 12, 14, 16, 18, 20, 22, 24, 26, 28, 30, 32, or 34, wherein the promotor and nucleic acid segment is endogenously expressed or encoded in an expression system.
2. The plant cell, plant seed, or plant of statement 1, wherein the promotor is a strong or inducible promotor.
3. The plant cell, plant seed, or plant of statement 1, wherein the promotor is a tissue-specific promotor.
4. The plant cell, plant seed, or plant of statement 1, wherein the nucleic acid segment encoding the RFS polypeptide is a RFS transgene in a non-native location in the genome of the plant cell, plant seed, or plant.
5. The plant cell, plant seed, or plant of statement 1, wherein the promoter is a CaMV 35S promoter, Cor15a promotor, 7DA2 promotor, CaMV 19S promoter, nos promoter, a Brachypodium PIN-like promotor, Adhl promoter, sucrose synthase promoter, a-tubulin promoter, ubiquitin promoter, actin promoter, cab promoter, PEPCase promoter, R gene complex promoter, poplar xylem-specific secondary cell wall specific cellulose synthase 8 promoter, cauliflower mosaic virus promoter, Z10 promoter from a gene encoding a 10 kDa zein protein, Z27 promoter from a gene encoding a 27 kDa zein protein, pea rbcS gene and the actin promoter from rice promoter, or phaseolin promoter.
6. The plant cell, plant seed, or plant of statement 1, wherein the promotor increases the endogenous expression of the RFS gene relative to the expression of an RFS gene without the heterologous promotor.
7. The plant cell, plant seed, or plant of statement 1, which is an agricultural crop species, forage crop species, a fiber crop species, or a biofuel species.
8. The plant cell, plant seed, or plant of statement 1, which is alfalfa, clover, soybeans, turnips, bromegrass, bluestem, fescue, a starch plant, a grain, a grass, a sugar producing plant, corn, wheat, soy, or rice.
9. The plant cell, plant seed, or plant of statement 8, wherein the grains are maize, wheat, barley, oats, rice, sorghum, millet, or rye.
10. The plant cell, plant seed, or plant of statement 8, wherein the grasses are switchgrass, prairie grass, wheat grass, sudangrass, sorghum, straw-producing plants, or miscanthus.
11. The plant cell, plant seed, or plant of statement 8, wherein the sugar producing plants are sugarcane or beets.
12. The plant cell, plant seed, or plant of statement 7, which is cotton or flax.
13. The plant cell, plant seed, or plant of statement 1 or 2, wherein overexpression of RFS delays flowering time by approximately thirty to approximately forty days as compared to plants not overexpressing RFS.
14. The plant cell, plant seed, or plant of statement 1 or 2, wherein overexpression of RFS delays flowering time by approximately fourteen to approximately twenty-nine days as compared to plant not overexpressing RFS.
15. The plant cell, plant seed, or plant of statement 1 or 2, wherein plants overexpressing RFS delay flowering approximately 62% longer than plants not overexpressing RFS.
16. The plant cell, plant seed, or plant of statement 1 or 2, wherein shoot dry weight of the plant overexpressing RFS increase by approximately 20 percent (%) as compared to plants not overexpressing RFS.
17. The plant cell, plant seed, or plant of statement 1 or 2, wherein plant cell, plant seed, or plant has increased tolerance to drought compared to a plant lacking the at least one heterologous promotor operably linked to the nucleic acid segment encoding the RFS polypeptide.
18. A method comprising growing a plant seed or plant comprising at least one heterologous promotor operably linked to a nucleic acid segment encoding a RFS polypeptide that has at least 95% sequence identity to any of SEQ ID NOS: 2, 3, 6, 8, 10, 12, 14, 16, 18, 20, 22, 24, 26, 28, 30, 32, or 34, wherein the promotor and nucleic acid segment is endogenously expressed or encoded in an expression system to thereby produce a mature plant.
19. The method of statement 18, further comprising harvesting biomass from the mature plant.
20. The method of statement 19, further comprising isolating glucan, oligosaccharides, disaccharides, monosaccharides, or a combination thereof from the biomass.
21. The method of statement 18, wherein the promotor is a strong or inducible promotor.
22. The method of statement 18, wherein the promotor is a tissue-specific promotor.
23. The method of statement 18, wherein the promoter is a CaMV 35S promoter, Cor15a promotor, 7DA2 promotor, CaMV 19S promoter, nos promoter, a Brachypodium PIN-like promotor, Adhl promoter, sucrose synthase promoter, a-tubulin promoter, ubiquitin promoter, actin promoter, cab promoter, PEPCase promoter. R gene complex promoter, poplar xylem-specific secondary cell wall specific cellulose synthase 8 promoter, cauliflower mosaic virus promoter, Z10 promoter from a gene encoding a 10 kDa zein protein, Z27 promoter from a gene encoding a 27 kDa zein protein, pea rbcS gene and the actin promoter from rice promoter, or phaseolin promoter.
24. The method of statement 18, further comprising exposing the plant seed or plant to abiotic stress.
25. The method of statement 18, wherein the abiotic stress comprises drought.
26. A method for generating a plant seed or plant that is drought tolerant, flowering-delayed, or both comprising:
   (a) introducing a non-native promotor into the genome of the plant seed or plant, wherein the non-native promotor is operably linked to a native RFS gene; or
   (b) transfecting a plant with an expression system comprising at least one heterologous promotor operably linked to a nucleic acid segment encoding a RFS polypeptide that has at least 95% sequence identity to any of SEQ ID NOS: 2, 3, 6, 8, 10, 12, 14, 16, 18, 20, 22, 24, 26, 28, 30, 32, or 34;
   wherein (a) or (b) increases the expression of the native RFS gene or the nucleic acid segment encoding a RFS polypeptide compared to a plant seed or plant without (a) or (b).

### REFERENCES

References in the present disclosure are as follows:
Ali U, Lu S, Fadlalla T, Iqbal S, Yue H, Yang B, Hong Y, Wang X, Guo L (2022) The functions of phospholipases and their hydrolysis products in plant growth, development and stress responses. Progress in Lipid Research: 101158.
Almagro Armenteros J J, Salvatore M, Emanuelsson O, Winther O, von Heijne G, Elofsson A, Nielsen H (2019) Detecting sequence signals in targeting peptides using deep learning. Life science alliance 2: e201900429.
Bari R, Dat Pant B, Stitt M, Scheible W-Rd (2006) PHO2, MicroRNA399, and PHR1 Define a Phosphate-Signaling Pathway in Plants. Plant Physiology 141: 988-999.
Bheemanahalli R, Sathishraj R, Manoharan M, Sumanth H N, Muthurajan R, Ishimaru T, Krishna J S V (2017) Is early morning flowering an effective trait to minimize heat stress damage during flowering in rice? Field Crops Research 203: 238-242.
Bouché F, Woods D P, Linden J, Li W, Mayer K S, Amasino R M, Périlleux C (2022) EARLY FLOWERING 3 and Photoperiod Sensing in Brachypodium distachyon. Frontiers in Plant Science 12.
Browse J, Roughan P G, Slack C R (1981) Light control of fatty acid synthesis and diurnal fluctuations of fatty acid composition in leaves. The Biochemical Journal 196: 347-354.
Bustos R, Castrillo G, Linhares F, Puga M I, Rubio V, Pérez-Pérez J, Solano R, Leyva A, Paz-Ares J (2010) A Central Regulatory System Largely Controls Transcriptional Activation and Repression Responses to Phosphate Starvation in Arabidopsis. PLOS Genetics 6: e1001102.
Castrillo G, Teixeira P J, Paredes S H, Law T F, de Lorenzo L, Feltcher M E, Finkel O M, Breakfield N W, Mieczkowski P, Jones C D, Paz-Ares J, Dangl J L (2017) Root microbiota drive direct integration of phosphate stress and immunity. Nature 543(7646):513-518.
Chen D, Wang S, Qi L, Yin L, Deng X (2018) Galactolipid remodeling is involved in drought-induced leaf senescence in maize. Environmental and Experimental Botany 150: 57-68.
Cho L H, Yoon J, An G (2017) The control of flowering time by environmental factors. Plant J 90: 708-719.
Chong L, Xu R, Huang P, Guo P, Zhu M, Du H, Sun X, Ku L, Zhu J-K, Zhu Y (2022) The tomato OST1-VOZ1 module regulates drought-mediated flowering. The Plant Cell.
Cockram J, Jones H, Leigh F J, O'Sullivan D, Powell W, Laurie D A, Greenland A J (2007) Control of flowering time in temperate cereals: genes, domestication, and sustainable productivity. Journal of Experimental Botany 58: 1231-1244.
Cook R, Lupette J, Benning C (2021) The Role of Chloroplast Membrane Lipid Metabolism in Plant Environmental Responses. Cells 10.
Czechowski T, Stitt M, Altmann T, Udvardi M K, Scheible W-R (2005) Genome-Wide Identification and Testing of Superior Reference Genes for Transcript Normalization in Arabidopsis. Plant Physiology 139: 5-17.
David L C, Girin T, Fleurisson E, Phommabouth E, Mahfoudhi A, Citerne S, Berquin P, Daniel-Vedele F, Krapp A, Ferrario-Méry S (2019) Developmental and physiological responses of Brachypodium distachyon to fluctuating nitrogen availability. Scientific reports 9: 3824-3824.
Dhindsa R S, Plumb-Dhindsa P, Thorpe T A (1981) Leaf Senescence: Correlated with Increased Levels of Membrane Permeability and Lipid Peroxidation, and Decreased Levels of Superoxide Dismutase and Catalase. Journal of Experimental Botany 32: 93-101.
Draper H H, Hadley M (1990) Malondialdehyde determination as index of lipid Peroxidation. In Methods in Enzymology, Vol 186. Academic Press, pp 421-431.
Earley K W, Haag J R, Pontes O, Opper K, Juehne T, Song K, Pikaard C S (2006) Gateway-compatible vectors for plant functional genomics and proteomics. Plant J 45: 616-629.
Espinoza-Corral R, 716 Schwenkert S, Lundquist P K (2021) Molecular changes of Arabidopsis thaliana plastoglobules facilitate thylakoid membrane remodeling under high light stress. The Plant Journal 106: 1571-1587.
Fisher L H C, Han J, Corke F M K, Akinyemi A, Didion T, Nielsen K K, Doonan J H, Mur L A J, Bosch M (2016) Linking Dynamic Phenotyping with Metabolite Analysis to Study Natural Variation in Drought Responses of Brachypodium distachyon. Frontiers in Plant Science 7.
Goodstein D M, Shu S, Howson R, Neupane R, Hayes R D, Fazo J, Mitros T, Dirks W, Hellsten U, Putnam N, Rokhsar D S (2011) Phytozome: a comparative platform for green plant genomics. Nucleic Acids Research 40: D1178-D1186.
Havko N E, Das M R, McClain A M, Kapali G, Sharkey T D, Howe G A (2020) Insect herbivory antagonizes leaf cooling responses to elevated temperature in tomato. Proceedings of the National Academy of Sciences 117: 2211-2217.
Hilbert D W, Swift D M, Detling J K, Dyer M I (1981) Relative growth rates and the grazing optimization hypothesis. Oecologia 51: 14-18.
Hong S-Y, Seo P J, Yang M-S, Xiang F, Park C-M (2008) Exploring valid reference genes for gene expression studies in Brachypodium distachyon by real-time PCR. BMC plant biology 8: 112-112.
Hong Y, Zhao J, Guo L, Kim S-C, Deng X, Wang G, Zhang G, Li M, Wang X (2016) Plant phospholipases D and C and their diverse functions in stress responses. Progress in Lipid Research 62: 55-74.
Hong Y, Zheng S, Wang X (2008) Dual Functions of Phospholipase Dal in Plant Response to Drought. Molecular Plant 1: 262-269.
Hooper C M, Castleden I R, Tanz S K, Aryamanesh N, Millar A H (2016) SUBA4: the interactive data analysis centre for Arabidopsis subcellular protein locations. Nucleic Acids Research 45: D1064-D1074.
Horton P, Park K-J, Obayashi T, Fujita N, Harada H, Adams-Collier C J, Nakai K (2007) WoLF PSORT: protein localization predictor. Nucleic acids research 35: W585-W587.
Jin S, Nasim Z, Susila H, Ahn J H (2021) Evolution and functional diversification of FLOWERING LOCUS TITERMINAL FLOWER 1 family genes in plants. Semin Cell Dev Biol 109: 20-30.
Kakei Y, Mochida K, Sakurai T, Yoshida T, Shinozaki K, Shimada Y (2015) Transcriptome analysis of hormone-induced gene expression in Brachypodium distachyon. Scientific Reports 5: 14476.
Kant S, Peng M, Rothstein S J (2011) Genetic Regulation by NLA and MicroRNA827 for Maintaining Nitrate-Dependent Phosphate Homeostasis in Arabidopsis. PLOS Genetics 7: e1002021.
Karimi M, Inzé D, Depicker A (2002) GATEWAY™ vectors for Agrobacterium-mediated plant transformation. Trends in Plant Science 7: 193-195.
Kazan K, Lyons R (2015) The link between flowering time and stress tolerance. Journal of Experimental Botany 67: 47-60.
Kim S-J, Zemelis-Durfee S, Wilkerson C, Brandizzi F (2017) In Brachypodium a complex signaling is actuated to protect cells from proteotoxic stress and facilitate seed filling. Planta 246: 75-89.
Kleinboelting N, Huep G, Kloetgen A, Viehoever P, Weisshaar B (2012) GABI-Kat SimpleSearch: new features of the Arabidopsis thaliana T-DNA mutant database. Nucleic Acids Res 40: D1211-1215.
Koda S, Onda Y, Matsui H, Takahagi K, Uehara-Yamaguchi Y, Shimizu M, Inoue K, Yoshida T, Sakurai T, Honda H, Eguchi S, Nishii R, Mochida K (2017) Diurnal Transcriptome and Gene Network Represented through Sparse Modeling in Brachypodium distachyon. Frontiers in Plant Science 8.
Kong X-M, Zhou Q, Zhou X, Wei B-D, Ji S-J (2019) Transcription factor CaNAC1 regulates low temperature-induced phospholipid degradation in green bell pepper. Journal of Experimental Botany 71: 1078-1091.
Kouzai Y, Kimura M, Yamanaka Y, Watanabe M, Matsui H, Yamamoto M, Ichinose Y, Toyoda K, Onda Y, 762 Mochida K, Noutoshi Y (2016) Expression profiling of marker genes responsive to the defence-associated phytohormones salicylic acid, jasmonic acid and ethylene in Brachypodium distachyon. BMC Plant Biology 16: 59.
Kumar S, Stecher G, Li M, Knyaz C, Tamura K (2018) MEGA X: Molecular Evolutionary Genetics Analysis across Computing Platforms. Molecular Biology and Evolution 35: 1547-1549.
Lambers H, Cawthray G R, Giavalisco P, Kuo J, Laliberté E, Pearse S J, Scheible W R, Stitt M, Teste F, Turner B L (2012) Proteaceae from severely phosphorus-impoverished soils extensively replace phospholipids with galactolipids and sulfolipids during leaf development to achieve a high photosynthetic phosphorus-use-efficiency. New Phytol 196: 1098-1108.
Letunic I, Bork P (2021) Interactive Tree Of Life (iTOL) v5: an online tool for phylogenetic tree display and annotation. Nucleic Acids Research 49: W293-W296.
Li H-m, Yu C-W (2018) Chloroplast Galactolipids: The Link Between Photosynthesis, Chloroplast Shape, Jasmonates. Phosphate Starvation and Freezing Tolerance. Plant and Cell Physiology 59: 1128-1134.
Li M, Welti R, Wang X (2006) Quantitative profiling of Arabidopsis polar glycerolipids in response to phosphorus starvation. Roles of phospholipases D zeta1 and D zeta2 in phosphatidylcholine hydrolysis and digalactosyldiacylglycerol accumulation in phosphorus-starved plants. Plant physiology 142: 750-761.
Li X (2011) Infiltration of Nicotiana benthamiana Protocol for Transient Expression via Agrobacterium. Bio-protocol 1: e95.
Lin Y-L, Tsay Y-F (2017) Influence of differing nitrate and nitrogen availability on flowering control in Arabidopsis. Journal of Experimental Botany 68: 2603-2609.
Liu H, Ding Y, Zhou Y, Jin W, Xie K, Chen L L (2017) CRISPR-P 2.0: An Improved CRISPR-Cas9 Tool for Genome Editing in Plants. Mol Plant 10: 530-532.
Liu X, Ma D, Zhang Z, Wang S, Du S, Deng X, Yin L (2019) Plant lipid remodeling in response to abiotic stresses. Environmental and Experimental Botany 165: 174-184.
Lundquist P K, Poliakov A, Giacomelli L, Friso G, Appel M, McQuinn R P, Krasnoff S B, Rowland E, Ponnala L, Sun Q, van Wik K J (2013) Loss of plastoglobule kinases ABC1K1 and ABC1K3 causes conditional degreening, modified prenyl-lipids, and recruitment of the jasmonic acid pathway. Plant Cell 25: 1818-1839.
Lv B, Nitcher R, Han X, Wang S, Ni F, Li K, Pearce S, Wu J, Dubcovsky J, Fu D (2014) Characterization of FLOWERING LOCUS T1 (FT1) Gene in Brachypodium and Wheat. PLOS ONE 9: e94171.
Mann D G, Lafayette P R, Abercrombie L L, King Z R, Mazarei M, Halter M C, Poovaiah C R, Baxter H, Shen H, Dixon R A, Parrott W A, Neal Stewart C, Jr. (2012) Gateway-compatible vectors for high throughput gene functional analysis in switchgrass (Panicum virgatum L.) and other monocot species. Plant Biotechnol J 10: 226-236.
Mitra S K, Walters B T, Clouse S D, Goshe M B (2009) An efficient organic solvent-based extraction method for the proteomic analysis of Arabidopsis plasma membranes. J Proteome Res 8: 2752-2767.
Nakamura Y (2013) Phosphate starvation and membrane lipid remodeling in seed plants. Prog Lipid Res 52: 43-50.
Nakamura Y (2018) Membrane Lipid Oscillation: An Emerging System of Molecular Dynamics in the Plant Membrane. Plant and Cell Physiology 59: 441-447.
Nakamura Y, Andrds F, Kanehara K, Liu Y-c, Drmann P, Coupland G (2014) Arabidopsis florigen F T binds to diurnally oscillating phospholipids that accelerate flowering. Nature Communications 5: 3553.
Naumann C, Miller J, Sakhonwasee S, Wieghaus A, Hause G, Heisters M, Birstenbinder K, Abel S (2019) The Local Phosphate Deficiency Response Activates Endoplasmic Reticulum Stress-Dependent Autophagy. Plant physiology 179: 460-476.
Nilsson L, Müller R, Nielsen T H (2007) Increased expression of the MYB-related transcription factor, PHR1, leads to enhanced phosphate uptake in Arabidopsis thaliana. Plant, Cell & Environment 30: 1499-1512.
Pant B D, Burgos A, Pant P, Cuadros-Inostroza A, Willmitzer L, Scheible W-R (2015) The transcription factor PHR1 regulates lipid remodeling and triacylglycerol accumulation in Arabidopsis thaliana during phosphorus starvation. Journal of Experimental Botany 66: 1907-1918.
Pfaff J, Denton A K, Usadel B, Pfaff C (2020) Phosphate starvation causes different stress responses inthe lipid metabolism of tomato leaves and roots. Biochim Biophys Acta Mol Cell Biol Lipids 1865: 158763.
Priest H D, Fox S E, Rowley E R, Murray J R, Michael T P, Mockler T C (2014) Analysis of global gene expression in Brachypodium distachyon reveals extensive network plasticity in response to abiotic stress. PloS one 9: e87499-e87499.
Qin Z, Bai Y, Muhammad S, Wu X, Deng P, Wu J, An H, Wu L (2019) Divergent roles of FT-like 9 in flowering transition under different day lengths in Brachypodium distachyon. Nature Communications 10: 812.
Qin Z, Wu J, Geng S, Feng N, Chen F, Kong X, Song G, Chen K, Li A, Mao L, Wu L (2017) Regulation of FT splicing by an endogenous cue in temperate grasses. Nature communications 8: 14320-14320.
Qiu D, Hu W, Zhou Y, Xiao J, Hu R, Wei Q, Zhang Y, Feng J, Sun F, Sun J, Yang G, He G (2021) TaASR1-D confers abiotic stress resistance by affecting ROS accumulation and ABA signalling in transgenic wheat. Plant Biotechnology Journal 19: 1588-1601.
Qu L, Chu Y-J, Lin W-H, Xue H-W (2021) A secretory phospholipase D hydrolyzes phosphatidylcholine to suppress rice heading time. PLOS Genetics 17: e1009905.
Raissig M T, Woods D P (2022) Chapter Two—The wild grass Brachypodium distachyon as a developmental model system. In B Goldstein, M Srivastava, eds, Current Topics in Developmental Biology, Vol 147. Academic Press, pp 33-71.
Raza A, Charagh S, Zahid Z, Mubarik M S, Javed R, Siddiqui M H, Hasanuzzaman M (2021) Jasmonic acid: a key frontier in conferring abiotic stress tolerance in plants. Plant Cell Reports 40: 1513-1541.
Ream T S, Woods D P, Schwartz C J, Sanabria C P, Mahoy J A, Walters E M, Kaeppler H F, Amasino R M (2013) Interaction of Photoperiod and Vernalization Determines Flowering Time of Brachypodium distachyon Plant Physiology 164: 694-709.
Riboni M, Galbiati M, Tonelli C, Conti L (2013) GIGANTEA Enables Drought Escape Response via Abscisic Acid-Dependent Activation of the Florigens and SUPPRESSOR O F OVEREXPRESSION O F CONSTANS1 Plant Physiology 162: 1706-1719.
Rubio V, Linhares F, Solano R, Martin A C, Iglesias J, Leyva A, Paz-Ares J (2001) A conserved MYB transcription factor involved in phosphate starvation signaling both in vascular plants and in unicellular algae. Genes Dev 15: 2122-2133.
Sang Y, Zheng S, Li W, Huang B, Wang X (2001) Regulation of plant water loss by manipulating the expression of phospholipase Da. The Plant Journal 28: 135-144.
Scholthof K-B G, Irigoyen S, Catalan P, Mandadi K K (2018) Brachypodium: A Monocot Grass Model Genus for Plant Biology. The Plant Cell 30: 1673-1694.
Schwartz C J, Doyle M R, Manzaneda A J, Rey P J, Mitchell-Olds T, Amasino R M (2010) Natural Variation of Flowering Time and Vernalization Responsiveness in Brachypodium distachyon. BioEnergy Research 3: 38-46.
Shavrukov Y, Kurishbayev A, Jatayev S, Shvidchenko V, Zotova L, Koekemoer F, de Groot S, Soole K, Langridge P (2017) Early Flowering as a Drought Escape Mechanism in Plants: How Can It Aid Wheat Production? Frontiers in Plant Science 8.
Shaw L M, Lyu B, Turner R, Li C, Chen F, Han X, Fu D, Dubcovsky J (2019) FLOWERING LOCUS T2 regulates spike development and fertility in temperate cereals. J Exp Bot 70: 193-204.
Shevchenko A, Wilm M, Vorm O, Mann M (1996) Mass spectrometric sequencing of proteins silver stained polyacrylamide gels. Anal Chem 68: 850-858.
Sun Q, Zybailov B, Majeran W, Friso G, Olinares P D, van Wijk K J (2009) PPDB, the Plant Proteomics Database at Cornell. Nucleic Acids Res 37: D969-974.
Sun Y, Jain A, Xue Y, Wang X, Zhao G, Liu L, Hu Z, Hu S, Shen X, Liu X, Ai H, Xu G, Sun S (2020) OsSQD1 at the crossroads of phosphate and sulfur metabolism affects plant morphology and lipid composition in response to phosphate deprivation. Plant, Cell & Environment 43: 1669-1690.
Susila H, Jurić S, Liu L, Gawarecka K, Chung K S, Jin S, Kim S-J, Nasim Z, Youn G, Suh M C, Yu H, Ahn J H (2021) Florigen sequestration in cellular membranes modulates temperature-responsive flowering. Science 373: 1137-1142.
Takáč T, Pechan T, Šamajová O, Šamaj J (2019) Proteomic Analysis of Arabidopsis pldα1 Mutants Revealed an Important Role of Phospholipase D Alpha 1 in Chloroplast Biogenesis. Front Plant Sci 10: 89.
Vigh L, Huitema H, Woltjes J, van Hasselt P R (1986) Drought stress-induced changes in the composition and physical state of phospholipids in wheat. Physiologia Plantarum 67: 92-96.
Wang L, Hu W, Feng J, Yang X, Huang Q, Xiao J, Liu Y, Yang G, He G (2016) Identification of the ASR gene family from Brachypodium distachyon and functional characterization of BdASR1 in response to drought stress. Plant Cell Reports 35: 1221-1234
Wang X (2005) Regulatory Functions of Phospholipase D and Phosphatidic Acid in Plant Growth, Development, and Stress Responses. Plant Physiology 139: 566-573.
Wang X, Guo L, Wang G, Li M (2014) PLD: Phospholipase Ds in Plant Signaling. In X Wang, ed, Phospholipases in Plant Signaling. Springer Berlin Heidelberg, Berlin, Heidelberg, pp 3-26.
Wang Y, Zhang X, Huang G, Feng F, Liu X, Guo R, Gu F, Zhong X, Mei X (2020) Dynamic changes in membrane lipid composition of leaves of winter wheat seedlings in response to PEG-induced water stress. BMC Plant Biology 20: 84.
Wang Z, Benning C (2011) Arabidopsis thaliana Polar Glycerolipid Profiling by Thin Layer Chromatography (TLC) Coupled with Gas-Liquid Chromatography (GLC). JoVE: e2518.
Wasternack C, Song S (2016) Jasmonates: biosynthesis, metabolism, and signaling by proteins activating and repressing transcription. Journal of Experimental Botany 68: 1303-1321.
Welti R, Li W, Li M, Sang Y, Biesiada H, Zhou H-E, Rajashekar C B, Williams T D, Wang X (2002) Profiling Membrane Lipids in Plant Stress Responses: Role of Phospholipase Dα In Freezing-Induced Lipid Changes In Arabidopsis. Journal of Biological Chemistry 277: 31994-32002.
Wijk K J v, Kessler F (2017) Plastoglobuli: Plastid Microcompartments with Integrated Functions in Metabolism, Plastid Developmental Transitions, and Environmental Adaptation. Annual Review of Plant Biology 68: 253-289.
Woods D, Dong Y, Bouche F, Bednarek R, Rowe M, Ream T, Amasino R (2019) A florigen paralog is required for short-day vernalization in a pooid grass. eLife 8: e42153.
Woods D P, Bednarek R, Bouché F, Gordon S P, Vogel J P, Garvin D F, Amasino R M (2017) Genetic Architecture of Flowering-Time Variation in Brachypodium distachyon. Plant physiology 173: 269-279.
Wu L, Liu D, Wu J, Zhang R, Qin Z, Liu D, Li A, Fu D, Zhai W, Mao L (2013) Regulation of FLOWERING LOCUS T by a MicroRNA in Brachypodium distachyon The Plant Cell 25: 4363-4377.
Xie K, Minkenberg B, Yang Y (2015) Boosting CRISPR/Cas9 multiplex editing capability with the endogenous tRNA-processing system. Proc Natl Acad Sci USA 112: 3570-3575
Yang Y, Zienkiewicz A, Lavell A, Benning C (2017) Coevolution of Domain Interactions in the Chloroplast TGD1, 2, 3 Lipid Transfer Complex Specific to Brassicaceae and Poaceae Plants. The Plant Cell 29:1500-1515.
Ying S, Blancaflor E B, Liao F, Scheible W-R (2022) A phosphorus-limitation induced, functionally conserved DUF506 protein is a repressor of root hair elongation in plants. New Phytologist 233: 1153-1171.
Ying, S., Scheible, W.-R. & Lundquist, P. K. (2023) A stress-inducible protein regulates drought tolerance and flowering time in Brachypodium and Arabidopsis. Plant Physiology, 191(1), 643-659.
Yoon J S, Kim J Y, Kim D Y, Seo Y W (2021) A novel wheat ASR gene, TaASR2D, enhances drought tolerance in Brachypodium distachyon. Plant Physiology and Biochemistry 159: 400-414.
Yoon J S, Kim J Y, Lee M B, Seo Y W (2019) Over-expression of the Brachypodium ASR gene, BdASR4, enhances drought tolerance in Brachypodium distachyon. Plant Cell Rep 38: 1109-1125.
Yu L, Zhou C, Fan J, Shanklin J, Xu C (2021) Mechanisms and functions of membrane lipid remodeling in plants. The Plant Journal 107: 37-53.
Zhang M, Barg R, Yin M, Gueta-Dahan Y, Leikin-Frenkel A, Salts Y, Shabtai S, Ben-Hayyim G (2005) Modulated fatty acid desaturation via overexpression of two distinct omega-3 desaturases differentially alters tolerance to various abiotic stresses in transgenic tobacco cells and plants. Plant J 44: 361-371.

The specific methods, devices and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

We claim:

1. A plant cell, plant seed, or plant comprising at least one heterologous promotor operably linked to a nucleic acid segment encoding a RFS polypeptide that has at least 95% sequence identity to any of SEQ ID NOS: 2, 3, 6, 8, 10, 12, 14, 16, 18, 20, 22, 24, 26, 28, 30, 32, or 34, wherein the promotor and nucleic acid segment is endogenously expressed or encoded in an expression system.

2. The plant cell, plant seed, or plant of claim 1, wherein the promotor is a strong or inducible promotor.

3. The plant cell, plant seed, or plant of claim 1, wherein the promotor is a tissue-specific promotor.

4. The plant cell, plant seed, or plant of claim 1, wherein the nucleic acid segment encoding the RFS polypeptide is a RFS transgene in a non-native location in the genome of the plant cell, plant seed, or plant.

5. The plant cell, plant seed, or plant of claim 1, wherein the promoter is a CaMV 35S promoter, Cor15a promotor, 7DA2 promotor, CaMV 19S promoter, nos promoter, a Brachypodium PIN-like promotor, Adhl promoter, sucrose synthase promoter, a-tubulin promoter, ubiquitin promoter, actin promoter, cab promoter, PEPCase promoter, R gene complex promoter, poplar xylem-specific secondary cell wall specific cellulose synthase 8 promoter, cauliflower mosaic virus promoter, Z10 promoter from a gene encoding a 10 kDa zein protein, Z27 promoter from a gene encoding a 27 kDa zein protein, pea rbcS gene and the actin promoter from rice promoter, or phaseolin promoter.

6. The plant cell, plant seed, or plant of claim 1, wherein the promotor increases the endogenous expression of the RFS gene relative to the expression of an RFS gene without the heterologous promotor.

7. The plant cell, plant seed, or plant of claim 1, which is an agricultural crop species, forage crop species, a fiber crop species, or a biofuel species.

8. The plant cell, plant seed, or plant of claim 1, which is alfalfa, clover, soybeans, turnips, bromegrass, bluestem, fescue, a starch plant, a grain, a grass, a sugar producing plant, corn, wheat, soy, or rice.

9. The plant cell, plant seed, or plant of claim 8, wherein the grains are maize, wheat, barley, oats, rice, sorghum, millet, or rye.

10. The plant cell, plant seed, or plant of claim 8, wherein the grasses are switchgrass, prairie grass, wheat grass, sudangrass, sorghum, straw-producing plants, or miscanthus.

11. The plant cell, plant seed, or plant of claim 8, wherein the sugar producing plants are sugarcane or beets.

12. The plant cell, plant seed, or plant of claim 7, which is cotton or flax.

13. The plant cell, plant seed, or plant of claims 1 or 2, wherein overexpression of RFS delays flowering time by approximately thirty to approximately forty days as compared to plants not overexpressing RFS.

14. The plant cell, plant seed, or plant of claims 1 or 2, wherein overexpression of RFS delays flowering time by approximately fourteen to approximately twenty-nine days as compared to plant not overexpressing RFS.

15. The plant cell, plant seed, or plant of claims 1 or 2, wherein plants overexpressing RFS delay flowering approximately 62% longer than plants not overexpressing RFS.

16. The plant cell, plant seed, or plant of claims 1 or 2, wherein shoot dry weight of the plant overexpressing RFS increase by approximately 20 percent (%) as compared to plants not overexpressing RFS.

17. The plant cell, plant seed, or plant of claims 1 or 2, wherein plant cell, plant seed, or plant has increased tolerance to drought compared to a plant lacking the at least one heterologous promotor operably linked to the nucleic acid segment encoding the RFS polypeptide.

18. A method comprising growing a plant seed or plant comprising at least one heterologous promotor operably linked to a nucleic acid segment encoding a RFS polypeptide that has at least 95% sequence identity to any of SEQ ID NOS: 2, 3, 6, 8, 10, 12, 14, 16, 18, 20, 22, 24, 26, 28, 30, 32, or 34, wherein the promotor and nucleic acid segment is endogenously expressed or encoded in an expression system to thereby produce a mature plant.

19. The method of claim 18, further comprising harvesting biomass from the mature plant.

20. The method of claim 19, further comprising isolating glucan, oligosaccharides, disaccharides, monosaccharides, or a combination thereof from the biomass.

21. The method of claim 18, wherein the promotor is a strong or inducible promotor.

22. The method of claim 18, wherein the promotor is a tissue-specific promotor.

23. The method of claim 18, wherein the promoter is a CaMV 35S promoter, Cor15a promotor, 7DA2 promotor, CaMV 19S promoter, nos promoter, a Brachypodium PIN-like promotor, Adhl promoter, sucrose synthase promoter, a-tubulin promoter, ubiquitin promoter, actin promoter, cab promoter, PEPCase promoter, R gene complex promoter, poplar xylem-specific secondary cell wall specific cellulose synthase 8 promoter, cauliflower mosaic virus promoter, Z10 promoter from a gene encoding a 10 kDa zein protein, Z27 promoter from a gene encoding a 27 kDa zein protein, pea rbcS gene and the actin promoter from rice promoter, or phaseolin promoter.

24. The method of claim 18, further comprising exposing the plant seed or plant to abiotic stress.

25. The method of claim 18, wherein the abiotic stress comprises drought.

26. A method for generating a plant seed or plant that is drought tolerant, flowering-delayed, or both comprising:
(a) introducing a non-native promotor into the genome of the plant seed or plant, wherein the non-native promotor is operably linked to a native RFS gene; or
(b) transfecting a plant with an expression system comprising at least one heterologous promotor operably linked to a nucleic acid segment encoding a RFS polypeptide that has at least 95% sequence identity to any of SEQ ID NOS: 2, 3, 6, 8, 10, 12, 14, 16, 18, 20, 22, 24, 26, 28, 30, 32, or 34;
wherein (a) or (b) increases the expression of the native RFS gene or the nucleic acid segment encoding a RFS polypeptide compared to a plant seed or plant without (a) or (b).
